SEQUENCE LISTING (14 sequences)

SEQ ID NO: 1 - DNA, 528 bp, Homo sapiens
atgcagcggagactggttcagcagtggagcgtcgcggtgttcctgctgagctacgcggtgccctcctgcgggcgctcggtggagggtctcagccgccgcctcaaaagagctgtgtctgaacatcagctcctccatgacaaggggaagtccatccaagatttacggcgacgattcttccttcaccatctgatcgcagaaatccacacagctgaaatcagagctacctcggaggtgtcccctaactccaagccctctcccaacacaaagaaccaccccgtccgatttgggtctgatgatgagggcagatacctaactcaggaaactaacaaggtggagacgtacaaagagcagccgctcaagacacctgggaagaaaaagaaaggcaagcccgggaaacgcaaggagcaggaaaagaaaaaacggcgaactcgctctgcctggttagactctggagtgactgggagtgggctagaaggggaccacctgtctgacacctccacaacgtcgctggagctcgattcacggtag

SEQ ID NO: 2 - protein, 175 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSR

SEQ ID NO: 3 - protein, 19 aa, Homo sapiens
KKKKGKPGKRKEQQKKKRR

SEQ ID NO: 4 - protein, 33 aa, Homo sapiens
TRSAWLDSGVTGSGLEGDHLSDTSTTSLELDSR

SEQ ID NO: 5 - protein, 24 aa, Homo sapiens
TRSAWLEGDHLSDTSTTSLELDSR

SEQ ID NO: 6 - protein, 23 aa, Homo sapiens
TRSAWLDSGVTGSGTTSLELDSR

SEQ ID NO: 7 - protein, 24 aa, Homo sapiens
TRSAWLDSGVTGSGLEGDHLSDTS

SEQ ID NO: 8 - protein, 33 aa, Homo sapiens
TRSAWLDSGVTGAGLEGDHLSDTATAALELDAR

SEQ ID NO: 9 - protein, 33 aa, Homo sapiens
TRSAWLDSGVTGAGLEGDHLSDTSTTSLELDSR

SEQ ID NO: 10 - protein, 33 aa, Homo sapiens
TRSAWLDSGVTGSGLEGDHLSDTATTSLELDSR

SEQ ID NO: 11 - protein, 33 aa, Homo sapiens
TRSAWLDSGVTGSGLEGDHLSDTSTASLELDSR

SEQ ID NO: 12 - protein, 33 aa, Homo sapiens
TRSAWLDSGVTGSGLEGDHLSDTSTTSLELDAR

SEQ ID NO: 13 - protein, 19 aa, Homo sapiens
KKKKGKPGKRKEQEKKKRR

SEQ ID NO: 14 - protein, 33 aa, Homo sapiens; identical to SEQ ID NO: 4

(19) United States
(12) Patent Application Publication
(10) Pub. No.: US 2010/0150909 A1
(43) Pub. Date: Jun. 17, 2010

(54) PTHrP-DERIVED MODULATORS OF SMOOTH MUSCLE PROLIFERATION

(76) Inventors: Andrew F. STEWART, Pittsburgh, PA (US); Nathalie FIASCHI-TAESCH, Pittsburgh, PA (US)

Correspondence Address: WILMERHALE/BOSTON, 60 STATE STREET, BOSTON, MA, 02109, US

(21) Appl. No.: 12/412,972

(22) Filed: Mar. 27, 2009

Related U.S. Application Data
(63) Continuation of application No. 11/058,384, filed on Feb. 15, 2005, now abandoned.
Application No. 11/058,384 is a continuation of application No. PCT/US03/25473, filed on Aug. 13, 2003.
(60) Provisional application No. 60/403,805, filed on Aug. 15, 2002.

(51) Int. Cl. A61K 38/29; C07K 14/635; C07H 21/04; C12N 15/63; C12N 5/10; C12N 7/01; C07K 16/26; A61K 39/395; A61K 31/7088; C12P 21/00; G01N 33/53; C12Q 1/68; A61K 35/76; G06G 7/58; A61P 21/00

(52) U.S. Cl. 424/133.1; 530/399; 536/23.51; 435/320.1; 435/325; 435/235.1; 514/12; 530/389.2; 530/388.24; 530/387.3; 514/44 R; 435/69.4; 435/7.1; 435/6; 436/501; 424/93.2; 703/11

(57) ABSTRACT

The present invention relates to the use of mutants of parathyroid hormone-related protein, to treat disorders associated with smooth muscle cells, and to inhibit the cellular activation and proliferation thereof. The method can be employed in diverse tissues to effect therapeutic and prophylactic relief for disorders and diseases manifested by activation of smooth muscle that can lead to excessive smooth muscle proliferation. For example, where employed in the vasculature, the inventive method can be used to treat restenosis following angioplasty.

### STATEMENT CONCERNING GOVERNMENT RIGHTS IN FEDERALLY-SPONSORED RESEARCH

Research involved in developing this invention was supported, in whole or in part, via Grant No. NIDDK R-O1 55081 from the United States National Institutes of Health. The Government of the United States of America may have certain rights in this application.

### BACKGROUND OF THE INVENTION

The phenotypic plasticity of smooth muscle cells permits this muscle cell lineage to subserve diverse functions in multiple tissues including the arterial wall, uterus, respiratory, liver, as well as the urogenital and digestive tracts. Accordingly, smooth muscle cell activation leading to excessive cell proliferation can cause a wide variety of pathological conditions. Such conditions include uterine fibroid tumors, prostatic hypertrophy, bronchial asthma, portal hypertension in cirrhosis, bladder disease, pulmonary and systemic arterial hypertension, atherosclerosis, and vascular restenosis after angioplasty, coronary heart disease, thrombosis, myocardial infarction, stroke, smooth muscle neoplasms such as leiomyoma and leiomyosarcoma of the bowel and uterus, and obliterative disease of vascular grafts and transplanted organs.

Atherosclerotic coronary and peripheral vascular disease place an enormous health and economic burden on populations living in developed countries. This is widely predicted to become more severe as the obesity and diabetes pandemic progresses, and as the population in developed countries ages. One of the mainstays of coronary artery disease treatment and myocardial infarction prevention is coronary artery angioplasty, and this technique is increasingly commonly applied to other arterial systems, including the peripheral vascular, renovascular and carotid arterial systems (Klugherz et al., Nat. Biotechnol. 18(11): 1181 (2000); Morice et al., N. Engl. J. Med. 346(23): 1773 (2002); Schnyder et al., N. Engl. J. Med. 345(22): 1593 (2001)). Angioplasty is highly effective, but is limited at present by both early and late failures. Coronary and peripheral vascular disease are increasingly treated using angioplasty approaches. Restenosis results in late failure in approximately 20-50% of patients undergoing angioplasty. Late failure is commonly due to arterial restenosis, a phenomenon which results from the proliferation and migration of arterial smooth muscle cells from the smooth muscle layer of the arterial wall, the media, into the lumen itself, where they form a new arterial layer termed the neointima. The neointima, composed of vascular smooth muscle (VSM) cells and the extracellular matrix they have secreted, expands with time and ultimately compromises the lumen of the angioplastied artery.

A need remains in the art for a method for the prevention and treatment of disorders manifested by altered smooth muscle growth.

### SUMMARY OF THE INVENTION

The present invention relates to smooth muscle cell modulating (SMCM) compositions that have the property of antagonizing the activation of smooth muscle cells and smooth muscle proliferation, as well as methods for the prophylactic and therapeutic treatment of a subject having disease states characterized by altered smooth muscle proliferation. More particularly, the compositions are related to parathyroid hormone-related protein mutants.

In aspect, the invention includes an SMCM compound comprising a parathyroid hormone-related protein mutant polypeptide wherein the compound, (a) lacks a functional nuclear localization signal; (b) overexpressing the compound in a vascular smooth muscle cell decreases the level of phosphorylated immunoreactive retinoblastoma polypeptide compared to the to the level of phosphorylated immunoreactive retinoblastoma polypeptide observed in the absence of the compound; and (c) overexpressing the compound in a vascular smooth muscle cell increases the level of immunoreactive p27kip1 polypeptide compared to the level of immunoreactive p27kip1 polypeptide observed in the absence of the compound, further including polynucleotides encoding such SMCM compounds. Also included are variants, analogs, homologs, or fragments of the polypeptide and polynucleotide sequences, and small molecules incorporating these.

In another aspect, the invention includes an SMCM compound comprising a parathyroid hormone-related protein mutant polypeptide wherein the compound has a functional nuclear localization signal and has one or more modified amino acids in the region of PTHrP(112-139). In one embodiment, the modification of amino acids in the region of PTHrP(112-139) is selected from the group consisting of a deletion, substitution, and derivatization, further including polynucleotides encoding such SMCM compounds. Also included are variants, analogs, homologs, or fragments of the polypeptide and polynucleotide sequences, and small molecules incorporating these.

In another embodiment, SMCM mutant polypeptide has a functional nuclear localization signal and a polypeptide selected from the group consisting of SEQ ID NOS:5, 6, 7, 8, 9, 10, 11, and 12.

In another embodiment, the invention includes an isolated nucleic acid molecule encoding the SMCM compounds. In yet another embodiment, the isolated nucleic acid is a vector, and the vector may optionally include a promoter sequence that can be operably linked to the nucleic acid, where the promoter causes expression of the nucleic acid molecule. In one embodiment, the promoter is inducible. In still another embodiment, the vector is transformed into a cell, such as a prokaryotic or eukaryotic cell, preferably a mammalian cell, or more preferably a human cell. In even another embodiment, the vector is a viral vector capable of infecting a mammalian cell and causing expression of a SMCM compound polypeptide in an animal infected with the virus. In another embodiment, the virus is adenovirus.

In another aspect, the invention includes a pharmaceutical composition having an SMCM compound, polynucleotide encoding an SMCM compound, a virus containing a polynucleotide encoding an SMCM compound, or an antibody, or fragment of an antibody that immunospecifically binds an SMCM compound, and a pharmaceutically acceptable carrier.

In one aspect, the invention includes a kit having in one or more containers, a pharmaceutical an SMCM composition, a polynucleotide encoding an SMCM compound, an antibody that immunospecifically binds an SMCM compound, a virus containing a polynucleotide encoding an SMCM compound and instructions for using the contents therein.

In yet another aspect, the invention includes an antibody to an SMCM compound or a fragment thereof that binds immunospecifically to an SMCM compound polypeptide. In one embodiment, the antibody is an antibody fragment, such as but not limited to an Fab, (Fab)2, Fv or Fc fragment. In another embodiment, the antibody or fragment thereof if is a monoclonal antibody. In even another embodiment, the antibody or fragment thereof is a humanized antibody. In still another embodiment, the invention includes an antibody or antibody fragment immunospecific to SMCM compound, and a pharmaceutically acceptable carrier. In yet another embodiment, the Invention includes a pharmaceutical composition having an SMCM compound polypeptide or the nucleic acid sequence of an SMCM compound, an antibody or antibody fragment, and a pharmaceutically-acceptable carrier.

In yet another aspect, the invention includes a method for preparing an SMCM compound, the method having the steps of culturing a cell containing a nucleic acid encoding an SMCM compound under conditions that provide for expression of the SMCM compound; and recovering the expressed SMCM compound.

In still another aspect, the Invention includes a method for determining the presence or amount of an SMCM compound in a sample, the method having the steps of providing the sample, contacting the sample with an antibody or antibody fragment that binds immunospecifically to the SMCM compound, and determining the presence or amount of the antibody bound to the SMCM compound, thereby determining the presence or amount of the SMCM compound in the sample.

In even another aspect, the invention includes a method for determining the presence or amount of the nucleic acid molecule encoding an SMCM compound in a sample, the method having the steps of providing the sample, contacting the sample with a nucleic acid probe that hybridizes to the nucleic acid molecule, and determining the presence or amount of the probe hybridized to the nucleic acid molecule, thereby determining the presence or amount of the nucleic acid molecule in the sample.

In another aspect, the invention includes a method of identifying a candidate compound that binds to an SMCM compound, the method having the steps of contacting the compound with the SMCM compound, and determining whether the candidate compound binds to the SMCM compound.

In one aspect, the invention includes a method of treating or preventing a smooth muscle proliferation-associated disorder, the method comprising administering to a subject in which such treatment or prevention is desired an SMCM compound in an amount sufficient to treat or prevent the smooth muscle proliferation-associated disorder in the subject. In one embodiment, the smooth muscle proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty. In still another embodiment, the invention includes a method of treating or preventing a smooth muscle proliferation-associated disorder, by administering to a subject in which such treatment or prevention is desired polynucleotide encoding an SMCM in an amount sufficient to treat or prevent the tissue differentiation factor-associated disorder in the subject. In one embodiment, the subject is a human subject. In another embodiment, the subject is an animal subject.

In yet another aspect, the invention includes a method of treating a pathological state in a mammal, the method comprising administering to the mammal an SMCM compound in an amount that is sufficient to alleviate the pathological state, wherein the compound is a compound having an amino acid sequence at least 90% identical to an SMCM compound.

In another aspect, the invention includes a method of treating a pathological state in a mammal, the method comprising administering to the mammal an antibody or fragment thereof immunospecific an SMCM compound, or a virus containing a polynucleotide encoding an SMCM compound in an amount sufficient to alleviate the pathological state. In one embodiment, the invention includes a method of treating a smooth muscle proliferation-associated disorder in a mammal, the method including administering to the mammal at least one compound which modulates the expression or activity of an SMCM compound. In yet another embodiment, the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty.

In yet another aspect, the invention provides a compound of for use in treating a smooth muscle cell proliferation-associated disorder, wherein the compound is a SMCM compound. In another aspect, the invention provides for the use of a compound for the manufacture of a medicament for treatment of a smooth muscle cell proliferation-associated disorder, wherein the compound is an SMCM compound. In another aspect, the invention provides a method of treating a pathological state in a mammal, the method comprising administering to the mammal a virus containing a polynucleotide encoding an SMCM in an amount sufficient to alleviate the pathological state.

In another aspect, the invention includes a method of identifying a candidate compound, which binds to a SMCM compound, the method having the steps of, providing a candidate compound, contacting the candidate compound with the SMCM compound under conditions where a complex is formed between the test compound and the SMCM compound, incubating the complex under conditions where co-crystals of the complex form, determining the structural atomic coordinates of the complex by x-ray diffraction, and modeling the structure of the complex to determine the binding of the candidate compound to the SMCM compound. In one embodiment the invention includes a crystalline preparation of a candidate compound and a SMCM compound. In another embodiment, the complex is not crystallized but the complex is subjected to nuclear magnetic spectroscopy or mass spectroscopy to determine binding of the complex.

In another aspect, the invention provides a device comprising a surface coated with a compound selected from the group consisting of an SMCM compound, a polynucleotide encoding an SMCM compound, a virus containing a polynucleotide encoding an SMCM compound, and an antibody or fragment of an antibody that binds immunospecifically to an SMCM compound, in one embodiment, the device is selected from the group consisting of a patch, stent, and catheter. In another aspect, the invention provides a method of treating a smooth muscle cell proliferating-associated disorder in a mammal, the method comprising contacting a subject with a device comprising a surface coated with a selected from the group consisting of an SMCM compound, a polynucleotide encoding an SMCM compound, a compound of virus containing a polynucleotide encoding an SMCM compound, and an antibody or fragment of an antibody that binds immunospecifically to an SMCM compound. In another embodiment, the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty. In another embodiment, the subject is a human.

### BRIEF DESCRIPTION OF THE DRAWINGS

The present invention will be further understood from the following description with reference to the figures, in which:

FIG. 1 is a schematic drawing of human wild-Type (WT-HA) PTHrP and the PTHrP-derived deletion mutants employed. Wild type PTHrP contains a signal peptide and a nuclear localization sequence (NLS). Each construct also contains a hemagglutinin (HA) tag. The numbers above the first construct indicate the location of basic amino acid clusters used in post-translational processing of PTHrP, and, in the case of the NLS, nuclear targeting of PTHrP.

FIG. 2 is a line graph depicting the effects of the PTHrP deletion mutants on the proliferation of A-10 vascular smooth muscle cells. The “n” adjacent to the title of each clone indicates the number of times each growth curve was performed; growth curves were performed three to four times on each of three clones derived from each construct. Error bars indicate standard error.

FIG. 3 is a schematic diagram of the amino acid sequence of the carboxy-terminus of PTHrP. Each of the carboxy-terminal regions selected for deletion are shown by the brackets, and the individual amino acids depicted using the single letter code. Bolded amino acid residues, Ser119, Ser130, Thr132, Ser133, and Ser138 indicate phosphorylation substrates for calmodulin kinase II (CKII) and/or protein kinase C(PKC).

FIG. 4 is a schematic drawing of human wild-Type (WT-HA) PTHrP and PTHrP-derived alanine substitution mutants, wherein each of the amino acids Ser119, Ser130, Thr132, Ser133, and Ser138 was mutagenized to an alanine (A)-encoding codon. The AC-HA construct (alanine combination or AC) has all five of these amino acids converted to alanine.

FIG. 5 is a line graph depicting the effects of the PTHrP alanine substitution mutants on the proliferation of A-10 vascular smooth muscle cells. The “n” adjacent to the title of each clone indicates the number of times each growth curve was performed; growth curves were performed three to four times on each of three clones derived from each construct. Error bars indicate standard error.

FIG. 6 are bar graphs showing the effect of select PTHrP mutations on the production of PTHrP(1-36) in stable A-10 vascular smooth muscle cell clones. Production of PTHrP(1-36) is expressed as immunoreactive protein level in the media (panel A) as detected by radioimmunoassay or expressed as picomoles of PTHrP(1-36) produced per milligram total cellular protein (pM/mg protein; panel b). Error bars indicate standard error. The dotted line indicates the radioimmunoassay detection limit at 0.5 pM for PTHrP(1-36).

FIG. 7 is a schematic drawing illustrating the mechanism of-ΔNLS PTHrP-mediated inhibition of vascular smooth muscle cell proliferation.

FIG. 8 illustrates the effect of PTHrP overexpression on retinoblastoma protein (pRb) phosphorylation. Panel A shows cell cycle analysis using standard flow cytometric analysis with propidium iodide, wherein the data are expressed graphically as cell number as a function of DNA content. Panel B is a Western blot showing the phosphorylation of pRb protein as detected by a pRb antibody (Pharmingen, San Diego, Calif.). In the bottom panel, beta tubulin is seen as a control for loading.

FIG. 9 illustrates the effect of the overexpression of an NLS deletion construct of PTHrP (NLS) on retinoblastoma protein (pRb) phosphorylation. Panel A shows cell cycle analysis using standard flow cytometric analysis with propidium iodide, wherein the data are expressed graphically as cell number as a function of DNA content. Panel B is a Western blot showing the phosphorylation of pRb protein as detected by a pRb antibody (Pharmingen, San Diego, Calif.). Beta tubulin used as a control for loading (bottom panel).

FIG. 10 illustrates the effect of the overexpression of an NLS deletion construct of PTHrP(NLS) on p27 protein expression. The level of cellular expression of immunoreactive p27 protein was determined by Western blot analysis using anti-p27 antibody. Actin was used as a control for sample loading (bottom panel). Immunoreactive p27 protein is expressed in control A-10 vascular smooth muscle cells. In contrast, overexpressing wild-type PTHrP in A-10 vascular smooth muscle cells (WT) inhibits immunoreactive p27 protein expression compared to the level of immunoreactive p27 protein expression observed in control A-10 vascular smooth muscle cells. On the other hand, overexpressing NLS PTHrP in A-10 vascular smooth muscle cells Increases immunoreactive p27 protein expression when compared to the level of immunoreactive p27 protein expression observed in control A-10 vascular smooth muscle cells.

FIG. 11 illustrated the transfection of A-10 smooth muscle cells (VSM) in using adenovirus expressing beta-galactosidase (ad-lacZ), wild-type PTHrP (adWT) or PTHrP deleted for the NLS. Replication-defective Ad5 adenovirus deleted for Ela and Elb, generously provided by Dr. Chris Newgard at Duke University was employed. Panel A shows photomicropgraphs of cultured rat A-10 VSM cells transfected with the ad-lacZ virus was at a multiplicity of infection (MOI) of 0 (left), 1250 (middle) or 2500 (right), respectively, for 15 minutes, and beta-galactosidase was visualized 48 hours later using standard methods. Panel B is a bar graph of immunoreactive PTHrP(1-36) (a.k.a., IRMA 1-36) production (pM) observed 48 h after transfection of A-10 VSM cells for 15 minutes at 2500 MOI with ad-lacZ, ad-WT, or adenovirus containing the NLS deletion construct of PTHrP (ad-ΔNLS) clones, respectively. The “n” values indicate the number of times the experiment was repeated, and the error bars indicate standard error. PTHrP in the conditioned medium was assessed using a PTHrP immunoradiometric assay with a detection limit of 0.5 pM for the PTHrP IRMA.

FIG. 12 show photomicrographs illustrating the effect of angioplasty and PTHrP gene therapy on rat carotid arterial neointima formation. Angioplasty and subsequent histologic analysis of the carotid sections was performed essentially as described by D'Andrea and coworkers (D'Andrea et al., Biotech. Histochem. 74(4):172-80 (1999)). Panel A shows normal control vessel. Panel B shows vessel two weeks following angioplasty. Panel C shows vessel treated with ad-lacZ, two weeks after angioplasty. Panel D shows vessel treated with adenovirus containing the NLS deletion construct of PTHrP (ad-ΔNLS), two weeks after angioplasty.

FIG. 13 illustrates the effect of angioplasty and PTHrP gene therapy on rat carotid arterial neointima formation. The “n” values indicate the number of times the experiment was repeated, and the error bars indicate standard error. Two weeks after angioplasty the treated carotid vessels and the 28 contralateral control carotid vessels were obtained and analyzed as described by D'Andrea and coworkers (D'Andrea et al., Biotech. Histochem. 74(4):172-80 (1999)). Briefly, the contralateral control artery (which received neither injury nor adenovirus treatment), and the balloon-injured artery with no adenovirus treatment (DMEM) or adenovirus treatment (ad-LacZ or ad-delta-NLS) were harvested and fixed in 4% paraformaldehyde for 48 h at 4° C., embedded in paraffin blocks, sectioned (5 gm), and stained either with hematoxylin and eosin or by Von Giesen method to reveal the internal and external elastic lamina. Images were acquired and analyzed for the cross-sectional areas of neointima and media using the NIH Image program, and the area ratio was calculated.

FIG. 14 illustrates the effect of angioplasty and PTHrP gene therapy on pig carotid arterial neointima formation. Two weeks after angioplasty the treated carotid vessels and the contralateral control carotid vessels were obtained and analyzed as described by D'Andrea and coworkers (D'Andrea et al., Biotech. Histochem. 74(4):172-80 (1999)). Briefly, the contralateral control artery (which received neither injury nor adenovirus treatment), and the balloon-injured artery with no adenovirus treatment (DMEM) or adenovirus treatment (ad-LacZ or ad-delta-NLS) were harvested and fixed in 4% paraformaldehyde for 48 h at 4° C., embedded in paraffin blocks, sectioned (5 gm), and stained either with hematoxylin and eosin or by Von Giesen method to reveal the internal and external elastic lamina. Images were acquired and analyzed for the cross-sectional areas of neointima and media using the NIH Image program, and the area ratio was calculated.

### DETAILED DESCRIPTION OF THE INVENTION

### I. Definitions

The term “parathyroid hormone-related protein” (PTHrP) encompasses naturally occurring PTHrP, as well as synthetic or recombinant PTHrP. Further, the term “parathyroid hormone-related protein” encompasses allelic variants, species variants, and conserved amino acid substitution variants. The term also encompasses full-length PTHrP as well as PTHrP fragments, including small peptidomimetic molecules having PTHrP-like bioactivity. PTHrP includes, but is not limited to, human PTHrP (hPTHrP), bovine PTHrP (bPTHrP), and rat PTHrP (rPTHrP)

“Basic amino acid,” as used herein, refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of a compound, as used herein, is a quantity sufficient to achieve a desired therapeutic and/or prophylactic effect, for example, an amount which results in the prevention of or a decrease in the symptoms associated with a disease that is being treated, e.g., the diseases associated with TGF-beta superfamily polypeptides listed above. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the SMCM compounds of the present Invention or polynucleotides encoding the SMCM compounds of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. Typically, an effective amount of a viral carrier, e.g., adenovirus, containing a polynucleotide construct encoding PTHrP or SMCM compound of the present invention sufficient for achieving a therapeutic or prophylactic effect, are administered at a concentration range from 1 pfu/ml to 1×10¹⁴ pfu/ml. In an another embodiment of the present invention, the effective amount of a viral carrier for achieving a therapeutic or prophylactic effect concentration range is administered at a concentration range from 1 pfu/ml to 1×1014 pfu/ml. The compounds of the present invention can also be administered in combination with each other, or with one or more additional therapeutic compounds.

The term “variant,” as used herein, refers to a compound that differs from the compound of the present invention, but retains essential properties thereof. A non-limiting example of this is a polynucleotide or polypeptide compound having conservative substitutions with respect to the reference compound commonly known as degenerate variants. Another non-limiting example of a variant is a compound that is structurally different, but retains the same active domain of the compounds of the present invention, for example, N-terminal or C-terminal extensions or truncations of a polypeptide compound. Generally, variants are overall closely similar, and in many regions, identical to the compounds of the present invention. Accordingly, the variants may contain alterations in the coding regions, non-coding regions, or both.

The term “sequence identity,” as used herein, refers to the degree to which two polynucleotide or polypeptide sequences are identical on a residue-by-residue basis over a particular region of comparison.

The term “percentage of sequence identity,” as used herein, is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity.

The term “substantial identity,” as used herein, denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

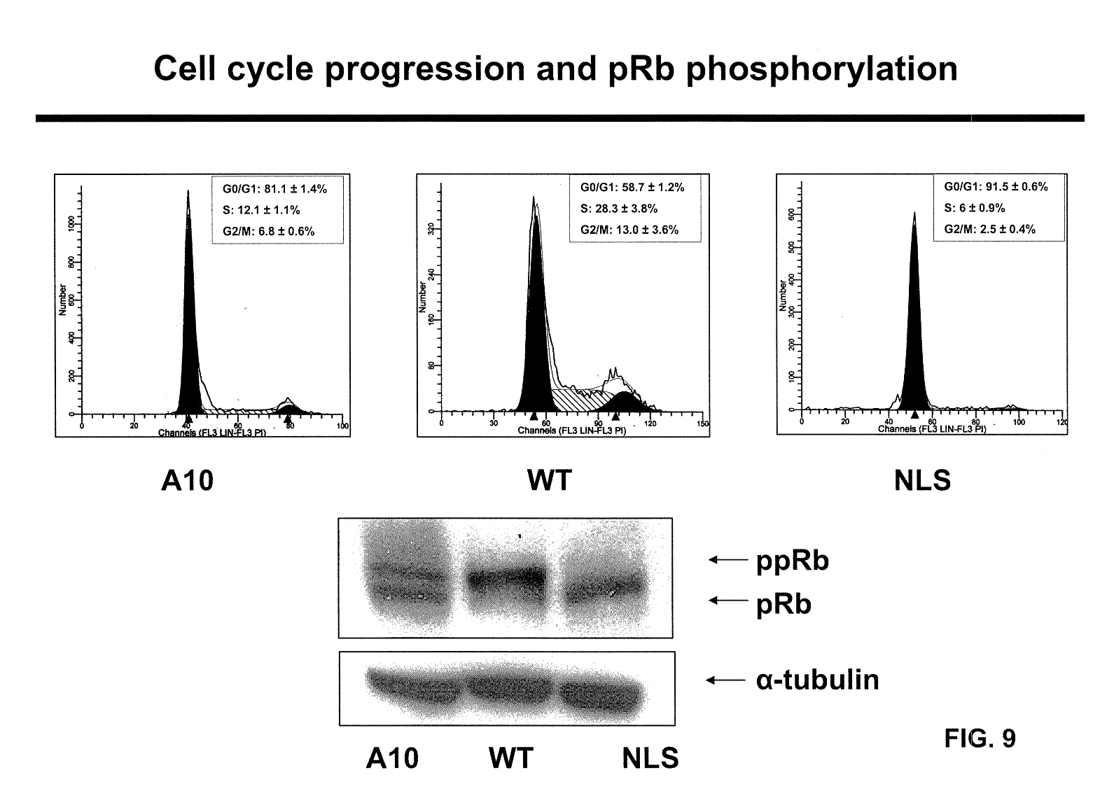

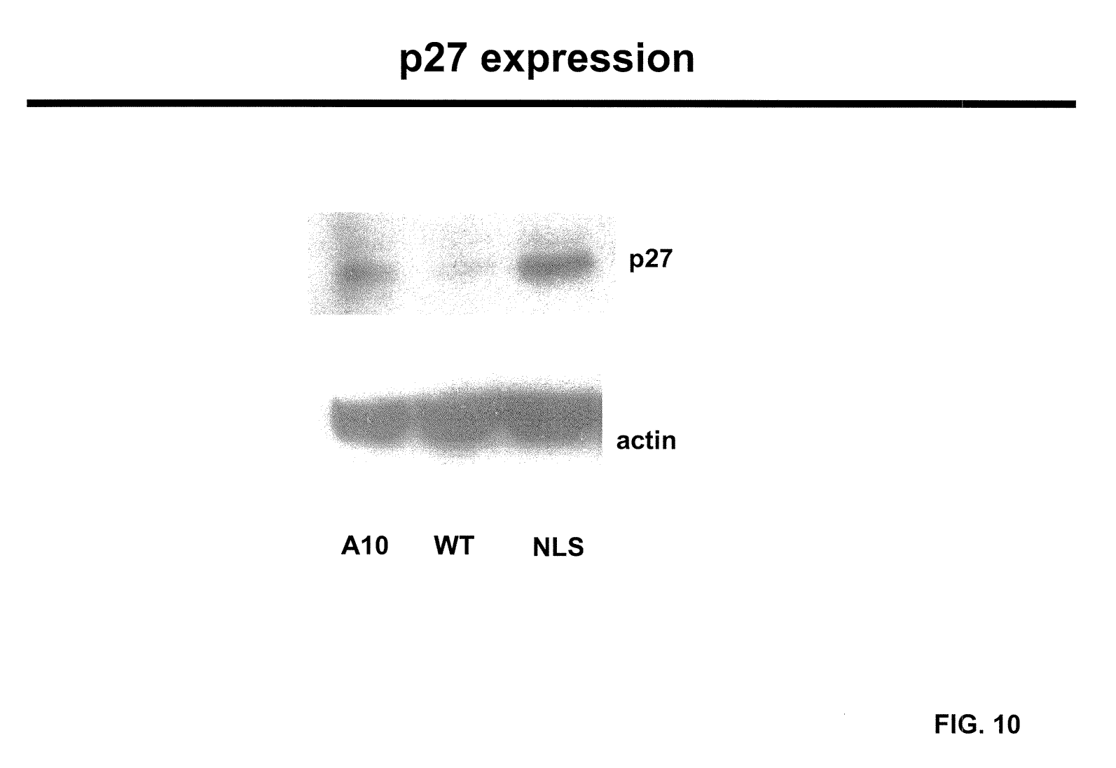

As used herein, the terms ΔNLS SMCM and ΔNLS PTHrP shall be construed to mean the same thing and are used interchangeable with the terms “NLS deletion construct of PTHrP” or “delta-NLS”. NLS, as used in FIG. 9 and FIG. 10 and with regard to discussion of those figures, represents A-10 cells overexpressing the NLS deletion construct of PTHrP. The term “ad-ΔNLS” is the NLS deletion construct of PTHrP expressed in an adenovirus.

The references cited throughout this application are incorporated herein by reference in their entireties.

### II. General

Parathyroid hormone-related protein (a.k.a., PTH-like adenylate cyclase-stimulating protein, PTHrP) was originally identified in the search for the humoral factor that causes humoral hypercalcemia of malignancy (Philbrick et al., Physiol Rev. 76(1): 127 (1996); Clemens et al., Br. J. Pharmacol. 134(6):1113 (2001)). PTHrP is produced in the arterial wall, is upregulated by vascular injury, by balloon distention and by vasoconstrictors, and acts as a vascular smooth muscle (VSM) relaxant. PTHrP is now known to be a widely distributed paracrine, autocrine, intracrine and endocrine factor which has diverse roles in regulating mammalian development, calcium ion transport, cellular proliferation and cell death (Philbrick et al., Physiol Rev. 76(1): 127 (1996); Clemens et al., Br. J. Pharmacol. 134(6):1113 (2001)). PTHrP also has a nuclear/nucleolar localization signal (NLS) in the 88-106 region. These roles are critical for survival. Indeed, disruption of the PTHrP gene results in embryonic lethality in mice (Karaplis and Kronenberg, Vitam. Horm. 52: 177 (1996)). One of the tissues that produces PTHrP is the VSM cell in the arterial wall (Ozekl et al., Arterioscler. Thromb. Vase. Biol. 16(4): 565 (1996); Nakayama et al., Biochem Biophys Res Commun. 200(2):1028 (1994); Massfelder and Helwig, Endocrinology 140(4): 1507 (1999); Qian et al., Endocrinology 140(4): 1826 (1999); Maeda et al., Endocrinology 140(4): 1815 (1999); Stuart et al., Am. J Physiol Endocrinol Metab. 279(1): E60 (2000)). PTHrP has been shown to be a potent vasodilator and hypotensive agent when injected systemically (Ozeki et al., Arterioscler. Thromb. Vase. Biol. 16(4): 565 (1996); Nakayama et al., Biochem Biophys Res Commun. 200(2):1028 (1994); Massfelder and Helwig, Endocrinology 140(4): 1507 (1999); Qian et al., Endocrinology 140(4): 1826 (1999); Maeda et al., Endocrinology 140(4): 1815 (1999); Stuart et al., Am. J Physiol Endocrinol Metab. 279(1): E60 (2000)). Moreover, overexpression of PTHrP or its receptor in the arterial wall of transgenic mice results in hypotension mediated by nitric oxide and by cyclic AMP (Ozeki et al., Arterioscler. Thromb. Vase. Biol. 16(4): 565 (1996); Nakayama et al., Biochem Biophys Res Commun. 200(2):1028 (1994); Massfelder and Helwig, Endocrinology 140(4): 1507 (1999); Qian et al., Endocrinology 140(4): 1826 (1999); Maeda et al., Endocrinology 140(4): 1815 (1999); Stuart et al., Am. J Physiol Endocrinol Metab. 279(1): E60 (2000)). In addition to its vasodilatory role, PTHrP also appears to regulate the rate of arterial smooth muscle proliferation both in vitro as well as in vivo (Massfelder et al., Proc Natl Acad Sci USA 94(25): 13630 (1997); de Miguel et al., Endocrinology 142(9): 4096 (2001)). Overexpression of PTHrP in vascular smooth muscle cells has been shown to stimulate proliferation (Massfelder et al., Proc Natl Acad Sci USA 94(25): 13630 (1997); de Miguel et al., Endocrinology 142(9): 4096 (2001)). In contrast, disruption of the PTHrP gene results in deceleration of the cell cycle in the arterial wall of embryonic mice (Massfelder et al., Proc Natl Acad Sci USA 94(25): 13630 (1997); de Miguel et al., Endocrinology 142(9): 4096 (2001)).

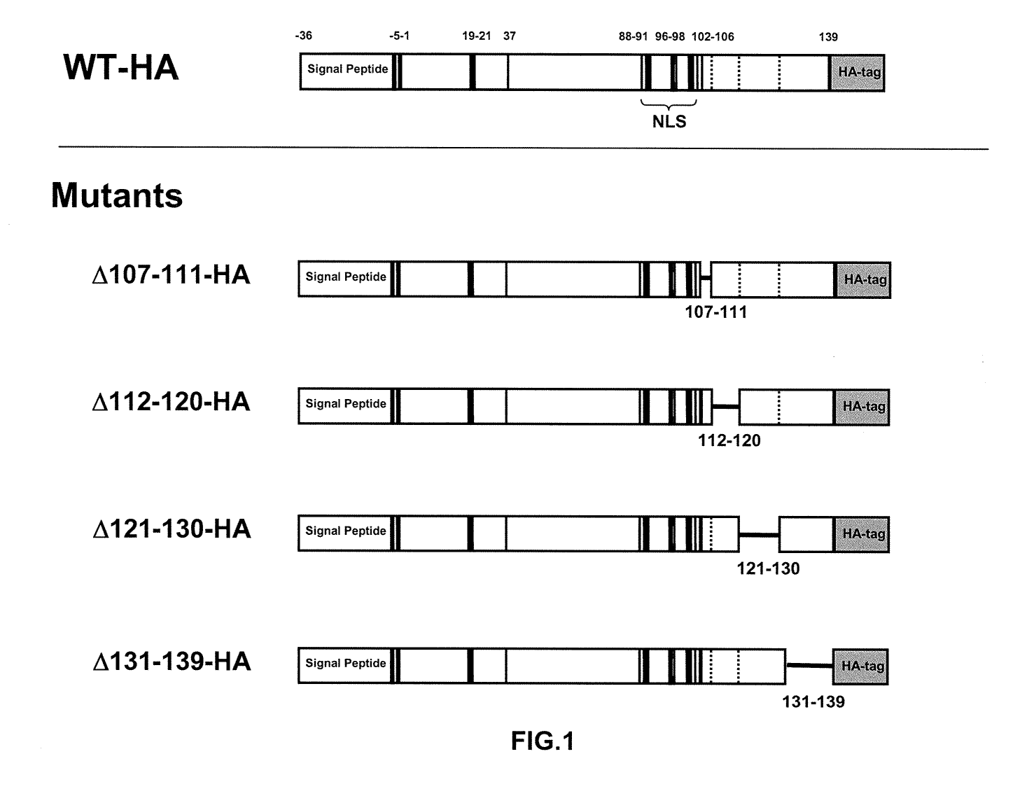

This ability of PTHrP to drive VSM proliferation depends, in part, on the presence of an intact nuclear localization signal, or NLS, a classical bipartite sequence of basic amino acids (FIG. 1) which interact with the components of the nuclear import machinery, including importin beta (Massfelder et al., Proc Natl Acad Sci USA 94(25): 13630 (1997); de Miguel et al., Endocrinology 142(9): 4096 (2001); Henderson et al., Mol Cell Biol. 15(8): 4064 (1995); Nguyen and Karaplis, J. Cell. Biochem. 70(2): 193 (1998)). The PTHrP mRNA contains two alternative translational initiation sites, with one directly upstream of a functional signal peptide that directs the PTHrP translation product to the secretory pathway, with resultant exocytosis. A second translation initiation site internal to the signal peptide can also be used (Henderson et al., Mol Cell Biol. 15(8): 4064 (1995); Nguyen and Karaplis, J. Cell. Biochem. 70(2): 193 (1998)). Use of this latter translational initiation site disrupts the signal peptide, and directs the PTHrP translation product to the cytosol, where, in concert with the NLS, it is directed to the nucleus. Therefore, it has been previously demonstrated that PTHrP can have either mitogenic or anti-mitogenic properties in VSM cells depending on whether the NLS is present or not: overexpression of wild type (WT) PTHrP results in marked increases in VSM cell number and tritiated thymidine incorporation in VSM cultures, associated with nuclear entry of PTHrP (Massfelder et al., Proc Natl Acad Scl USA 94(25): 13630 (1997); de Miguel et al., Endocrinology 142(9): 4096 (2001)). On the other hand, overexpression PTHrP containing a deleted NLS (delta-NLS-PTHrP) results in the opposite: marked slowing of proliferation in VSM cells, and failure of PTHrP to gain access to the nucleus (Massfelder et al., Proc Natl Acad Sci USA 94(25): 13630 (1997); de Miguel et al., Endocrinology 142(9): 4096 (2001)).

PTHrP is involved in the neointimal response to angioplasty. PTHrP has been repeatedly demonstrated to be upregulated in arterial smooth muscle in angioplastied coronary arteries (Philbrick et al., Physiol Rev. 76(1): 127 (1996); Clemens et al., Br. J. Pharmacol. 134(6): 1113 (2001)). Further, PTHrP is also upregulated in atherosclerotic human coronary arteries resected at the time of coronary bypass grafting (Ozeki et al., Arterioscler. Thromb. Vase. Biol. 16(4): 565 (1996); Nakayama et al., Biochem Biophys Res Commun. 200(2):1028 (1994); Massfelder and Helwig, Endocrinology 140(4): 1507 (1999); Qian et al., Endocrinology 140(4): 1826 (1999); Maeda et al., Endocrinology 140(4): 1815 (1999); Stuart et al., Am. J Physiol Endocrinol Metab. 279(1): E60 (2000)). Moreover, PTHrP is able to bidirectionally regulate VSM cell proliferation (Massfelder et al., Proc Natl Acad Sci USA 94(25): 13630 (1997); de Miguel et al., Endocrinology 142(9): 4096 (2001)). The following examples are provided for illustrative purposes only, and are in no way intended to limit the scope of the present invention.

### III. Compositions of the Invention

A. Smooth Muscle Cell Modulating Compounds

The present invention provides smooth muscle cell modulating (SMCM) compounds that are derivatives of PTHrP and modulate smooth muscle cell function. Such SMCM compositions are suitable for administration to a subject where it is desirable to inhibit the cellular activation of smooth muscle, e.g., but not limited to, phosphorylation of retinoblasoma protein (pRp), modulation of p27kip1 protein, and binding of PTHrP to PTHrP target molecule(s), that can lead to smooth muscle cell proliferation. Pathological conditions such as uterine fibroid tumors, prostatic hypertrophy, bronchial asthma, portal hypertension in cirrhosis, pulmonary and systemic arterial hypertension, atherosclerosis, and vascular restenosis after angioplasty are thought to be the result of smooth muscle cell activation and excessive smooth muscle cell proliferation. Accordingly, the SMCM compounds of the present invention are useful for the prophylactic treatment, or therapeutic treatment of disorders manifested by smooth muscle activation and excessive smooth muscle proliferation, e.g., uterine fibroid tumors, prostatic hypertrophy, bronchial asthma, portal hypertension in cirrhosis, pulmonary and systemic arterial hypertension, bladder disease, atherosclerosis, and vascular restenosis after angioplasty. It is also an object of the Invention to provide for compounds that are partial antagonists and smooth muscle activation and excessive smooth muscle cell proliferation.

The SMCM compounds of the present invention are polypeptide derivatives of PTHrP, a 139-plus amino acid protein, elaborated by a number of human and animal tumors and other tissues. Also contemplated within the scope of the present invention are the polynucleotides that encode the SMCM compounds of the present invention.

The structure of the gene for human PTHrP contains multiple exons and multiple sites for alternate splicing patterns during formation of the mRNA. Protein products of 139, 141, and 173 amino acids are produced, and other molecular forms may result from tissue-specific cleavage at accessible internal cleavage sites. A nucleotide sequence encoding human PTHrP (BT007178 [gi:30583194]; SEQ ID NO:1) is shown in Table 1.

TABLE 1
atgcagcggagactggttcagcagtggagcgtcgcggtgttcctgctgag
ctacgcggtgccctcctgcgggcgctcggtggagggtctcagccgccgcc
tcaaaagagctgtgctgaacatcagctcctccatgacaaggggaagtcca
tccaagatttacggcgacgattcttocttcaccatctgatcgcagaaatc
cacacagctgaaatcagagctacctcggaggtgtcccctaactccaagcc
ctctcccaacacaaagaaccaccccgtccgatttgggtctgatgatgagg
gcagatacctaactcaggaaactaacaaggtggagacgtacaaagagcag
ccgctcaagacacctgggaagaaaaagaaaggcaagcccgggaaacgcaa
ggagcaggaaaagaaaaaacggcgaactcgctctgcctggttagactctg
gagtgactgggagtgggctagaaggggaccacctgtctgacacctccaca
acgtcgctggagctcgattcacggtag

An amino acid sequence of a human PTHrP polypeptide (AAP35842 [gi:30583195]]; SEQ ID NO:2) is shown in Table 2.

TABLE 2
MQRRLVQQWSVAVFLLSYAVPSCGRSVEGLSRRLKRAVSEHQLLHDKGKS
IQDLRRRFFLHHLIAEIHTAEIRATSEVSPNSKPSPNTKNHPVRFGSDDE
GRYLTQETNKVETYKEQPLKTPGKKKKGKPGKKKEQEKKKRRTRSAWLDS
GVTGSGLEGDHLSDTSTTSLELDSR

PTHrP polypeptides containing a nuclear localization signal (NLS) can be directed to the nucleus of cells. The NLS in PTHrP is a bipartite, multibasic arrangement of amino acids, e.g., arginine and lysine. NLS sequences In human PTHrP are highlighted in bold text in Table 2 and shown in Table 3.

TABLE 3

| | |
|---|---|
| KKKKgKpgKRKeqqKKKRR | (SEQ ID NO: 3) |
| KKKKGKPGKRKEQEKKKRR | (SEQ ID NO: 13) |

In one embodiment the SMCM compounds of the present invention lack a functional PTHrP NLS (ΔNLS SMCM). That is, these SMCM compounds are not directed to the nucleus of an SMCM-expressing cell via the recognition of an NLS. Variants, analogs, homologs, or fragments of these compounds, such as species homologs, are also included in the present invention, as well as degenerate forms thereof. The ΔNLS SMCM compounds can contain one, two, three or more amino acid substitutions at any amino acid residues within the NLS sequence, e.g., SEQ ID NOS:3 and 13. Substitutions can contain natural amino acids, non-natural amino acids, d-amino acids and 1-amino acids, and any combinations thereof. The ΔNLS SMCM compounds can have deletion of one or more amino acids of the NLS of SEQ ID NOS:3 and 13.

The carboxy-terminus sequence of PTHrP(107-139) is shown in Table 4 (SEQ ID NO:4; deMiguel et al., Endocrinology 142: 4096-4105 (2001)). Carboxy-terminus amino acids 107 through 111 are highly conserved among species and are highlighted in bold text. The underlined serine and threonine amino acid residues, e.g., Ser119, Ser130, Thr132, Ser133, and Ser138, are potential sites for post-translational modification, e.g., but not limited to, phosphorylation, O-glycosylation, e.g., N-acetylgalactosamine, and acylation.

TABLE 4

| | |
|---|---|
| TRSAWLDSGVTGSGLEGDHLSDTSTTSLELDSR | (SEQ ID NO: 4) |

In another embodiment, the SMCM compounds are modified in the carboxy-terminus region of PTHrP(112-139) (ΔC-terminus SMCM). Variants, analogs, homologs, or fragments of these compounds, such as species homologs, are also included in the present invention, as well as degenerate forms thereof. The ΔC-terminus SMCM compounds of the present invention contain a functional NLS. The ΔC-terminus SMCM compounds can have deletion of one or more amino acids in the PTHrP(112-139) region. Representative deletions in the PTHrP(112-139) region include, but are not limited to, the following polypeptide sequences summarized in Table 5.

TABLE 5

| Deletion | SEQUENCE | SEQ ID NO. |
|---|---|---|
| Δ112-120 | TRSAWLEGDHLSDTSTTSLELDSR | 5 |
| Δ121-130 | TRSAWLDSGVTGSGTTSLELDSR | 6 |
| Δ131-139 | TRSAWLDSGVTGSGLEGDHLSDTS | 7 |

The ΔC-terminus SMCM compounds can contain one, two, three or more amino acid substitutions at any amino acid residues within the PTHrP(112-139) region. The substitutions can contain natural amino acids, non-natural amino acids, d-amino acids and 1-amino acids, and any combinations thereof. Representative polypeptides with single, double, or triple amino acid substitutions in the PTHrP(112-139) region include, but are not limited to, the following polypeptide sequences summarized in Table 6. The substituted residues are underlined.

TABLE 6

| | | SEQ |
|---|---|---|
| Deletion | SEQUENCE | ID NO. |
| AC | TRSAWLDSGVTGAGLEGDHLSDTATAALELDAR | 8 |
| A119 | TRSAWLDSGVTGAGLEGDHLSDTSTTSLELDSR | 9 |
| A130 | TRSAWLDSGVTGSGLEGDHLSDTATTSLELDSR | 10 |
| A132 | TRSAWLDSGVTGSGLEGDHLSDTSTASLELDSR | 11 |
| A138 | TRSAWLDSGVTGSGLEGDHLSDTSTTSLELDAR | 12 |

As noted above, the SMCM compounds of the present invention can contain natural amino acids, non-natural amino acids, d-amino acids and 1-amino acids, and any combinations thereof. In certain embodiments, the compounds of the invention can include commonly encountered amino acids which are not genetically encoded. These non-genetically encoded amino acids include, but are not limited to, β-alanine (β-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Om); citrulline (Cit); t-butylalanine (t-BuA); t-butylglycine (t-BuG); N-methylisoleucine (Melle); phenylglycine (Phg); cyclohexylalanine (Cha); norleucine (Nle); 2-naphthylalanine (2-Nal); 4-chlorophenylalanine (Phe(4-CI)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanlne (Phe(pNH2)); N-methyl valine (MeVal); homocysteine (hCys) and homoserine (hSer). Non-naturally occurring variants of the SMCM compounds may be produced by mutagenesis techniques or by direct synthesis. The SMCM compound of the present Invention may be capped on the N-terminus or the C-terminus or on both the N-terminus and the C-terminus.

The SMCM compounds of the present invention may be pegylated, or modified, e.g., branching, at any amino acid residue containing a reactive side chain, e.g., lysine residue.

In one embodiment, a SMCM compound includes an analog or homolog of SEQ ID Nos:2-12. Compounds of the present invention Include those with homology to SEQ ID Nos:2-12, for example, preferably 50% or greater amino acid identity, more preferably 75% or greater amino acid identity, and even more preferably 90% or greater amino acid identity.

Sequence identity can be measured using sequence analysis software (Sequence Analysis Software Package of the Genetics Computer Group, University of Wisconsin Biotechnology Center, 1710 University Avenue, Madison, Wis. 53705), with the default parameters therein.

In the case of polypeptide sequences, which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the invention are peptides having mutated sequences such that they remain homologous, e.g., in sequence, in structure, in function, and in antigenic character or other function, with a polypeptide having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

The invention also provides for compounds having altered sequences including insertions such that the overall amino acid sequence is lengthened, while the compound still retains the appropriate smooth muscle cell modulating property, e.g., inhibition of the cellular activation of smooth muscle, e.g., but not limited to, phosphorylation of retinoblasoma protein (pRp), modulation of p27kip1 protein, and binding of PTHrP to PTHrP target molecule(s), that can lead to smooth muscle cell proliferation. In certain embodiments, one or more amino acid residues within the NLS region or PTHrP(112-139) carboxy-terminus region are replaced with other amino acid residues having physical and/or chemical properties similar to the residues they are replacing. Preferably, conservative amino acid substitutions are those wherein an amino acid is replaced with another amino acid encompassed within the same designated class, as will be described more thoroughly below. Insertions, deletions, and substitutions are appropriate where they do not abrogate the functional properties of the compound. Functionality of the altered compound can be assayed according to the in vitro and in vivo assays described below that are designed to assess the SMCM-like properties of the altered compound.

B. SMCM Nucleic Acid Sequences

The compounds of the present invention Include one or more polynucleotides encoding the SMCM polypeptides, including degenerate variants thereof. Accordingly, nucleic acid sequences capable of hybridizing at low stringency with any nucleic acid sequences encoding SMCM compounds of the present invention are considered to be within the scope of the invention. For example, for a nucleic acid sequence of about 20-40 bases, a typical prehybridization, hybridization, and wash protocol is as follows: (1) prehybridization: incubate nitrocellulose filters containing the denatured target DNA for 3-4 hours at 55° C. in 5×Denhardt's solution, 6×SSC (20×SSC consists of 175 g NaCl, 88.2 g sodium citrate in 800 ml H2O adjusted to pH. 7.0 with 10 N NaOH), 0.1% SDS, and 100 mg/ml denatured salmon sperm DNA, (2) hybridization: incubate filters in prehybridization solution plus probe at 42° C. for 14-48 hours, (3) wash; three 15 minutes washes in 6×SSC and 0.1% SDS at room temperature, followed by a final 1-1.5 minutes wash in 6×SSC and 0.1% SDS at 55° C. Other equivalent procedures, e.g., employing organic solvents such as formamide, are well known in the art. Standard stringency conditions are well characterized in standard molecular biology cloning texts. See, for example Molecular Cloning A Laboratory Manual, 2nd Ed., ed., Sambrook, Fritsch, and Maniatis (Cold Spring Harbor Laboratory Press: 1989); DNA Cloning, Volumes I and II (D. N. Glovered., 1985); Oligonucleotide synthesis (M. J. Gait ed., 1984); Nucleic Acid Hybridization (B. D. Hames & S. J. Higgins eds, 1984).

The invention also encompasses allelic variants of the same, that is, naturally occurring alternative forms of the isolated polynucleotides that encode PTHrP polypeptides that are identical, homologous or related to those encoded by the polynucleotides. Alternatively, non-naturally occurring variants may be produced by mutagenesis techniques or by direct synthesis techniques well known in the art.

C. SMCM Recombinant Expression Vectors

Another aspect of the invention includes vectors containing one or more nucleic acid sequences encoding an SMCM compound. For recombinant expression of one or more the polypeptides of the invention, the nucleic acid containing all or a portion of the nucleotide sequence encoding the polypeptide is inserted into an appropriate cloning vector, or an expression vector (i.e., a vector that contains the necessary elements for the transcription and translation of the inserted polypeptide coding sequence) by recombinant DNA techniques well known in the art and as detailed below.

In general, expression vectors useful in recombinant DNA techniques are often in the form of plasmids. In the present specification, “plasmid” and “vector” can be used interchangeably as the plasmid is the most commonly used form of vector. However, the invention is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression in that subject of a compound (See Becker et al., Meth. Cell Biol. 43: 161-89 (1994)).

The recombinant expression vectors of the invention comprise a nucleic acid encoding an SMCM compound in a form suitable for expression of the nucleic acid in a host cell, which means that the recombinant expression vectors include one or more regulatory sequences, selected on the basis of the host cells to be used for expression that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g. In an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides, encoded by nucleic acids as described herein (e.g., SMCM compounds and SMCM-derived fusion polypeptides, etc.).

D. SMCM-Expressing Host Cells

Another aspect of the invention pertains to SMCM-expressing host cells, which contain a nucleic acid encoding one or more SMCM compounds. The recombinant expression vectors of the invention can be designed for expression of SMCM compounds in prokaryotic or eukaryotic cells. For example, SMCM compounds can be expressed in bacterial cells such as Escherichia coli (E. coli), insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase. The SMP2 promoter is useful in the expression of polypeptides in smooth muscle cells (Qian et al., Endocrinology 140(4): 1826 (1999)).

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., Gene 69:301-315 (1988)) and pET 11d (Studier et al., GENE EXPRESSION TECHNOLOGY: METHODS IN ENZYMOLOGY 185, Academic Press, San Diego, Calif. (1990)60-89).

One strategy to maximize recombinant polypeptide expression in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in the expression host, e.g., E. coli (see, e.g., Wada, et al., Nucl. Acids Res. 20: 2111-2118 (1992)). Such alteration of nucleic acid sequences of the invention can be carried out by standard DNA synthesis techniques.

In another embodiment, the SMCM expression vector is a yeast expression vector. Examples of vectors for expression in yeast Saccharomyces cenvisae include pYepSed (Baldari, et al., EMBO J. 6: 229-234 (1987)), pMFa (Kurjan and Herskowitz, Cell 30: 933-943 (1982)), pJRY88 (Schultz et al., Gene 54: 113-123 (1987)), pYES2 (InVitrogen Corporation, San Diego, Calif.), and picZ (InVitrogen Corp. San Diego, Calif.). Alternatively, SMCM can be expressed in insect cells using baculovirus expression vectors. Baculovirus vectors available for expression of polypeptides in cultured insect cells (e.g., SF9 cells) include the pAc series (Smith, et al., Mol. Cell. Biol. 3: 2156-2165 (1983)) and the pVLseries (Lucklow and Summers, Virology 170: 31-39 (1989)).

In yet another embodiment, a nucleic acid of the invention is expressed In mammalian cells using a mammalian expression vector. Examples of mammalian expression vectors include pCDM8 (Seed, Nature 329: 842-846 (1987)) and pMT2PC (Kaufman, et al., EMBO J. 6: 187-195 (1987)). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., Genes Dev. 1: 268-277 (1987)), lymphoid-specific promoters (Calame and Eaton, Adv. Immunol. 43: 235-275 (1988)), in particular promoters of T cell receptors (Winoto and Baltimore, EMBO J. 8: 729-733 (1989)) and immunoglobulins (Banerji, et al., Cell 33: 729-740 (1983); Queen and Baltimore, Cell 33: 741-748 (1983)), neuron-specific promoters (e.g., the neurofilament promoter; Byrne and Ruddle, Proc. Natl. Acad. Sci. USA 86: 5473-5477 (1989)), pancreas-specific promoters (Edlund, et al., Science 230: 912-916 (1985)), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249: 374-379 (1990)) and the α-fetoprotein promoter (Campes and Tilghman, Genes Dev. 3: 537-546 (1989)).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to a SMCM mRNA. Regulatory sequences operatively linked to a nucleic acid cloned in the antisense orientation can be chosen that direct the continuous expression of the antisense RNA molecule In a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, SMCM can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest Various selectable markers include those that confer resistance to drugs, such as G418, hygromycin and methotrexate. Nucleic acid encoding a selectable marker can be introduced into a host cell on the same vector as that encoding SMCM or can be introduced on a separate vector. Cells stably transfected with the introduced nucleic acid can be identified by drug selection (e.g., cells that have incorporated the selectable marker gene will survive, while the other cells die).

A host cell that includes a compound of the Invention, such as a prokaryotic or eukaryotic host cell in culture, can be used to produce (i.e., express) recombinant SMCM. In one embodiment, the method comprises culturing the host cell of invention (into which a recombinant expression vector encoding SMCM has been introduced) in a suitable medium such that SMCM is produced. In another embodiment, the method further comprises the step of isolating SMCM from the medium or the host cell. Purification of recombinant polypeptides is well-known in the art and include ion-exchange purification techniques, or affinity purification techniques, for example with an antibody to the compound. Methods of creating antibodies to the compounds of the present invention are discussed below.

### IV. Preparation of SMCM Compounds

A. Peptide Synthesis of SMCM Compounds

In one embodiment, a SMCM compound can be synthesized chemically using standard peptide synthesis techniques, e.g., solid-phase or solution-phase peptide synthesis. That is, the SMCM compounds are chemically synthesized, for example, on a solid support or in solution using compositions and methods well known in the art. See, e.g., Fields, G. B. (1997) Solid-Phase Peptide Synthesis. Academic Press, San Diego.

The SMCM compound may be prepared by either Fmoc (base labile protecting group) or -Boc (acid labile a-amino protecting group) peptide synthesis. Following synthesis, SMCM compound can then be rendered substantially free of chemical precursors or other chemicals by an appropriate purification scheme using standard polypeptide purification techniques for example, ion exchange chromatography, affinity chromatography, reverse-phase HPLC, e.g., using columns such as C-18, C-8, and C-4, size exclusion chromatography, chromatography based on hydrophobic Interactions, or other polypeptide purification method.

B. Production of SMCM Compound Using Recombinant DNA Techniques

In another embodiment, SMCM compounds are produced by recombinant DNA techniques, for example, overexpression of the compounds in bacteria, yeast, baculovirus or eukaryotic cells yields sufficient quantities of the compounds. Purification of the compounds from heterogeneous mixtures of materials, e.g., reaction mixtures or cellular lysates or other crude fractions, is accomplished by methods well known in the art, for example, ion exchange chromatography, affinity chromatography or other polypeptide purification methods. These can be facilitated by expressing the SMCM compounds described as fusions to a cleavable or otherwise inert epitope or sequence. The choice of an expression system as well as methods of purification are well known to skilled artisans.

The polynucleotides provided by the present invention can be used to express recombinant compounds for analysis, characterization or therapeutic use; as markers for tissues in which the corresponding compound is preferentially expressed (either constitutively or at a particular stage of tissue differentiation or development or in disease states).

For recombinant expression of one or more the compounds of the invention, the nucleic acid containing all or a portion of the nucleotide sequence encoding the peptide may be inserted into an appropriate expression vector (i.e., a vector that contains the necessary elements for the transcription and translation of the inserted peptide coding sequence). In some embodiments, the regulatory elements are heterologous (i.e., not the native gene promoter). Alternately, the necessary transcriptional and translational signals may also be supplied by the native promoter for the genes and/or their flanking regions.

A variety of host vector systems may be utilized to express the peptide coding sequence(s). These include, but are not limited to: (i) mammalian cell systems that are infected with vaccinia virus, adenovirus, and the like; (ii) insect cell systems infected with baculovirus and the like; (iii) yeast containing yeast vectors or (iv) bacteria transformed with bacteriophage, DNA, plasmid DNA, or cosmid DNA. Depending upon the host vector system utilized, any one of a number of suitable transcription and translation elements may be used.

Promoter/enhancer sequences within expression vectors may utilize plant, animal, insect, or fungus regulatory sequences, as provided in the invention. For example, promoter/enhancer elements from yeast and other fungi can be used (e.g., the GAL4 promoter, the alcohol dehydrogenase promoter, the phosphoglycerol kinase promoter, the alkaline phosphatase promoter). Alternatively, or in addition, they may include animal transcriptional control regions, e.g., (i) the Insulin gene control region active within pancreatic cells (see, e.g., Hanahan, et al., Nature 315: 115-122 (1985)); (ii) the immunoglobulin gene control region active within lymphoid cells (see, e.g., Grosschedl, et al., Cell 38: 647-658 (1984)); (iii) the albumin gene control region active within liver (see, e.g., Pinckert, et al., Genes and Dev 1: 268-276 (1987)); (iv) the myelin basic polypeptide gene control region active within brain oligodendrocyte cells (see, e.g., Readhead, et al., Cell 48: 703-712 (1987)); and (v) the gonadotropin releasing hormone gene control region active within the hypothalamus (see, e.g., Mason, et al., Science 234: 1372-1378 (1986)), and the like.

Expression vectors or their derivatives include, e.g. human or animal viruses (e.g., vaccinia virus or adenovirus); insect viruses (e.g., baculovirus); yeast vectors; bacteriophage vectors (e.g., lambda phage); plasmid vectors and cosmid vectors.

A host cell strain may be selected that modulates the expression of inserted sequences of interest, or modifies or processes expressed peptides encoded by the sequences in the specific manner desired. In addition, expression from certain promoters may be enhanced in the presence of certain inducers in a selected host strain; thus facilitating control of the expression of a genetically engineered compounds. Moreover, different host cells possess characteristic and specific mechanisms for the translational and post translational processing and modification (e.g., glycosylation, phosphorylation, and the like) of expressed peptides. Appropriate cell lines or host systems may thus be chosen to ensure the desired modification and processing of the foreign peptide is achieved. For example, peptide expression within a bacterial system can be used to produce an unglycosylated core peptide; whereas expression within mammalian cells ensures “native” glycosylation of a heterologous peptida.

C. Preparation of SMCM-Derived Chimeric or Fusion Polypeptide Compounds

A SMCM-derived chimeric or fusion polypeptide compound of the invention can be produced by standard recombinant DNA techniques known in the art. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS 1N MOLECULAR BIOLOGY, John Wiley & Sons, 1992). Moreover, many expression vectors are commercially available that already encode a fusion moiety (e.g., a GST polypeptide). A SMCM-encoding nucleic acid can be cloned into such an expression vector such that the fusion moiety is linked in-frame to the SMCM encoding nucleic acid sequence.

D. Preparation of SMCM Compound Polypeptide Libraries

In addition, libraries of fragments of the nucleic acid sequences encoding SMCM compounds can be used to generate a population of SMCM fragments for screening and subsequent selection of variants of a SMCM compound. In one embodiment, a library of coding sequence fragments can be generated by treating a double stranded PCR fragment of a nucleic acid sequence encoding SMCM compound with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with 51 nuclease, and ligating the resulting fragment library into an expression vector. By this method, expression libraries can be derived which encode N-terminal, C-terminal, and internal fragments of various sizes of the SMCM compounds.

Various techniques are known in the art for screening gene products of combinatorial libraries made by point mutations or truncation, and for screening cDNA libraries for gene products having a selected property. Such techniques are adaptable for rapid screening of the DNA libraries generated by the combinatorial mutagenesis of SMCM compound. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify SMCM compound variants. See, e.g., Arkin and Yourvan, Proc. Natl. Acad. Sci. USA 89: 7811-7815 (1992); Delgrave, et al., Polypeptide Engineering 6:327-331 (1993).

A library of SMCM compounds can also be produced by, for example, enzymatically ligating a mixture of synthetic oligonucleotides into gene sequences such that a degenerate set of potential SMCM compound sequences are is expressible as individual polypeptides, or alternatively, as a set of larger fusion polypeptides (e.g., for phage display) containing the set of SMCM compound sequences therein. There are a variety of methods that can be used to produce libraries of potential SMCM variant compounds from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SMCM compound sequences. Methods for synthesizing degenerate oligonucleotides are well-known within the art. See, e.g., Narang Tetrahedron 39: 3 (1983); Itakura, et al., Annu. Rev. Biochem. 53: 323 (1984); Itakura, et al., Science 198:1056 (1984); Ike, et al., Nucl. Acids Res. 11:477 (1983).

E. Anti-SMCM Compound Antibodies

The invention provides compounds including polypeptides and polypeptide fragments suitable for use as immunogens to raise anti-SMCM compound antibodies. The compounds can be used to raise whole antibodies and antibody fragments, such as Fv, Fab or (Fab)₂, that bind immunospecifically to any of the SMCM compounds of the invention, including bispecific or other multivalent antibodies.

An isolated SMCM polypeptide compound, or a portion or fragment thereof, can be used as an immunogen to generate antibodies that bind to SMCM compound or PTHrP polypeptides or PTH polypeptides using standard techniques for polyclonal and monoclonal antibody preparation. The full-length PTHrP polypeptides can be used or, alternatively, the invention provides for the use of compounds including SMCM compounds or SMCM fragments as immunogens. The SMCM compound peptides comprises at least 4 amino acid residues of the amino acid sequence shown in SEQ ID NO:4, and encompasses an epitope of SMCM compound such that an antibody raised against the peptide forms a specific immune complex with PTHrP polypeptide, PTH polypeptide, or SMCM compound. Preferably, the antigenic peptide comprises at least 5, 8, 10, 15, 20, or 30 amino acid residues. Longer antigenic peptides are sometimes preferable over shorter antigenic peptides, depending on use and according to methods well known to those skilled in the art.

In certain embodiments of the invention, at least one epitope encompassed by the antigenic peptide is a region of SMCM compound that is located on the surface of the polypeptide (e.g., a hydrophilic region). As a means for targeting antibody production, hydropathy plots showing regions of hydrophilicity and hydrophobicity can be generated by any method well known in the art, including, for example, the Kyte Doolittle or the Hopp Woods methods, either with or without Fourier transformation (see, e.g., Hopp and Woods, Proc. Nat. Acad. Sci. USA 78: 3824-3828 (1981); Kyte and Doolittle 1157: 105-142 (1982), each incorporated herein by reference in their entirety).

As disclosed herein, SMCM compounds or derivatives thereof, can be utilized as immunogens in the generation of antibodies that immunospecifically-bind these polypeptide components. In a specific embodiment, antibodies to human SMCM polypeptides are disclosed. Various procedures known within the art can be used for the production of polyclonal or monoclonal antibodies to a SMCM compound polypeptide sequence of SEQ ID NO:4-12, or a derivative, fragment, analog or homolog thereof. Some of these polypeptides are discussed below.

For the production of polyclonal antibodies, various suitable host animals (e.g., rabbit, goat, mouse or other mammal) can be immunized by injection with the native polypeptide, or a synthetic variant thereof, or a derivative of the foregoing. An appropriate immunogenic preparation can contain, for example, recombinantly-expressed SMCM compound or a chemically-synthesized SMCM compound. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory compounds. If desired, the antibody molecules directed against PTHrP or SMCM compound can be isolated from the mammal (e.g., from the blood) and further purified by well known techniques, such as polypeptide A chromatography to obtain the IgG fraction.

For preparation of monoclonal antibodies directed towards a particular SMCM compound, or derivatives, fragments, analogs or homologs thereof, any technique that provides for the production of antibody molecules by continuous cell line culture can be utilized. Such techniques include, but are not limited to, the hybridoma technique (see, e.g., Kohler & Milstein Nature 256: 495-497 (1975)); the trioma technique; the human B-cell hybridoma technique (see, e.g., Kozbor, et al., Immunol. Today 4; 72 (1983)) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies can be utilized in the practice of the invention and can be produced by using human hybridomas (see, e.g., Cote, et al., Proc Natl Acad Sci USA 80: 2026-2030 (1983)) or by transforming human B-cells with Epstein Barr Virus in vitro (see, e.g. Cole, et al., 1985. In: MONOCLONAL ANTIBODIES AND CANCER THERAPY, Alan R. Liss, Inc., pp. 77-96). Each of the above citations is Incorporated herein by reference in their entirety. Synthetic dendromeric trees can be added a reactive amino acid side chains, e.g., lysine to enhance the immunogenic properties of SMCM compounds. Also, CPG-dinucleotide technique can be used to enhance the immunogenic properties of SMCM compounds.

According to the invention, techniques can be adapted for the production of single-chain antibodies specific to a SMCM compound (see, e.g., U.S. Pat. No. 4,946,778). In addition, methods can be adapted for the construction of Fab expression libraries (see, e.g., Huse, et al., Science 246: 1275-1281 (1989)) to allow rapid and effective identification of monoclonal Fab fragments with the desired specificity for a SMCM compound, e.g., a polypeptide or derivatives, fragments, analogs or homologs thereof. Non-human antibodies can be “humanized” by techniques well known In the art. See, e.g., U.S. Pat. No. 5,225,539. Antibody fragments that contain the idiotypes to a SMCM compound can be produced by techniques known in the art including, but not limited to: (i) an F(ab′)₂ fragment produced by pepsin digestion of an antibody molecule; (ii) an Fab fragment generated by reducing the disulfide bridges of an F(ab′)2 fragment; (iii) an Fab fragment generated by the treatment of the antibody molecule with papain and a reducing compound; and (iv) Fv fragments.

Additionally, recombinant anti-SMCM compound antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. Nos. 4,816,567; 5,225,539; European Patent Application No. 125,023; Better, et al., Science 240: 1041-1043 (1988); Liu, et al., Proc. Natl. Acad. Sci. USA 84: 3439-3443 (1987); Liu, et al., J. Immunol. 139: 3521-3526 (1987); Sun, et al., Proc. Natl. Acad. Sci. USA 84: 214-218 (1987); Nishimura. et al., Cancer Res. 47: 999-1005 (1987); Wood, et al., Nature 314:446-449 (1985); Shaw, et al., J. Natl. Cancer Inst. 80: 1553-1559 (1988)); Morrison Science 229:1202-1207 (1985); Oi, et al. BioTechniques 4:214 (1986); Jones, et al., Nature 321: 552-525 (1986); Verhoeyan, et al., Science 239: 1534 (1988); and Beidler, et al., J. Immunol. 141: 4053-4060 (1988). Each of the above citations are incorporated herein by reference in their entirety.

In one embodiment, methods for the screening of antibodies that possess the desired specificity to the SMCM compounds include, but are not limited to, enzyme-linked immunosorbent assay (ELISA) and other immunologically-mediated techniques known within the art. In a specific embodiment, selection of antibodies that are specific to a particular domain of a SMCM compound polypeptide is facilitated by generation of hybridomas that bind to the fragment of a SMCM compound polypeptide possessing such a domain. Thus, antibodies that are specific for a desired domain within a SMCM compound, or derivatives, fragments, analogs or homologs thereof, are also provided herein.

Anti-SMCM compound antibodies can be used in methods known within the art relating to the localization and/or quantitation of a PTHrP polypeptide or SMCM compound (e.g., for use in measuring levels of the PTHrP polypeptide or SMCM compound within appropriate physiological samples, for use in diagnostic methods, for use in imaging the polypeptide, and the like). In a given embodiment, antibodies for SMCM compounds, or derivatives, fragments, analogs or homologs thereof, that contain the antibody derived binding domain, are utilized as pharmacologically-active compounds (hereinafter “Therapeutics”).

An anti-SMCM compound antibody (e.g., monoclonal antibody) can be used to isolate a SMCM compound or PTHrP polypeptide by standard techniques, such as affinity chromatography or immunoprecipitation. An anti-SMCM compound antibody can facilitate the purification of natural PTHrP polypeptide from cells and of recombinantly-produced SMCM compound expressed in host cells. Moreover, an anti-SMCM compound antibody can be used to detect PTHrP polypeptide or SMCM compounds (e.g., in a cellular lysate or cell supernatant) in order to evaluate the abundance and pattern of expression of the PTHrP polypeptide or SMCM compound. Anti-SMCM compound antibodies can be used diagnostically to monitor polypeptide levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include: ³²P, ¹²⁵I, ¹³¹I, ³⁵P, ³³P, ¹⁴C, ¹³C, or ³H.

### V. Biological Activity of SMCM Compounds

A. PTHrP Biological Actions

PTHrP exerts important developmental influences on fetal bone development and in adult physiology. A homozygous knockout of the PTHrP gene (or the gene for the PTH receptor) in mice causes a lethal deformity in which animals are born with severe skeletal deformities resembling chondrodysplasia. Many different cell types produce PTHrP, including brain, pancreas, heart, lung, mammary tissue, placenta, endothelial cells, and smooth muscle. In fetal animals, PTHrP directs transplacental calcium transfer, and high concentrations of PTHrP are produced in mammary tissue and secreted into milk. Human and bovine milk, for example, contain very high concentrations of the hormone; the biologic significance of the latter is unknown. PTHrP may also play a role in uterine contraction and other biologic functions, still being clarified in other tissue sites.

Because PTHrP shares a significant homology with PTH in the critical amino terminus, it binds to and activates the PTH/PTHrP receptor, with effects very similar to those seen with PTH. However, PTHrP, not PTH, appears to be the predominant physiologic regulator of bone mass, with PTHrP being essential for the development of full bone mass. Demonstrating this, conditional gene knockout strategies, employing mice in which the PTHrP gene was disrupted in osteoblasts prevented the production of PTHrP locally within adult bone, but which had normal PTH levels in adult bone. Absent PTHrP, and these mice developed osteoporosis demonstrating that osteoblast-derived PTHrP exerts anabolic effects in bone by promoting osteoblast function. See, Karaplis, A. C. “Conditional Knockout of PTHrP in osteoblasts Leads to Premature Osteoporosis.” Abstract 1052, Annual Meeting of the American Society for Bone and Mineral Research, September 2002, San Antonio, Tex. J Bone Mineral Res, (Suppl 1), pp S138, 2002, incorporated by reference.

The 500-amino-acid PTH/PTHrP receptor (also known as the PTH1 receptor) belongs to a subfamily of GCPR that includes those for glucagon, secretin, and vasoactive intestinal peptide. The extracellular regions are involved in hormone binding, and the intracellular domains, after hormone activation, bind G protein subunits to transduce hormone signaling into cellular responses through stimulation of second messengers.

A second PTH receptor (PTH2 receptor) is expressed in brain, pancreas, and several other tissues. Its amino acid sequence and the pattern of its binding and stimulatory response to PTH and PTHrP differ from those of the PTH1 receptor. The PTH/PTHrP receptor responds equivalents to PTH and PTHrP, whereas the PTH2 receptor responds only to PTH. The endogenous ligand of this receptor appears to be tubular infundibular peptide 39 or TIP 39. The physiological significance of the PTH2 receptor-TIP-39 system remains to be defined. Recently, a 39-amino-acid hypothalamic peptide, tubular infundibular peptide (TIP-39), has been characterized and is a likely natural ligand of the PTH2 receptor.

The PTH1 and PTH2 receptors can be traced backward in evolutionary time to fish. The zebrafish PTH1 and PTH2 receptors exhibit the same selective responses to PTH and PTHrP as do the human PTH1 and PTH2 receptors. The evolutionary conservation of structure and function suggests unique biologic roles for these receptors. G proteins of the Gs class link the PTH/PTHrP receptor to adenylate cyclase, an enzyme that generates cyclic AMP, leading to activation of protein kinase A. Coupling to G proteins of the Gq class links hormone action to phospholipase C, an enzyme that generates inositol phosphates (e.g., IP3) and DAG, leading to activation of protein kinase C and intracellular calcium release. Studies using the cloned PTH/PTHrP receptor confirm that it can be coupled to more than one G protein and second-messenger kinase pathway, apparently explaining the multiplicity of pathways stimulated by PTH and PTHrP. Incompletely characterized second-messenger responses (e.g., MAP kinase activation) may be independent of phospholipase C or adenylate cyclase stimulation (the latter, however, is the strongest and best characterized second messenger signaling pathway for PTH and PTHrP).

The details of the biochemical steps by which an increased intracellular concentration of cyclic AMP, IP3, DAG, and intracellular Ca2+ lead to ultimate changes in ECF calcium and phosphate ion translocation or bone cell function are unknown. Stimulation of protein kinases (A and C) and intracellular calcium transport is associated with a variety of hormone-specific tissue responses. These responses include inhibition of phosphate and bicarbonate transport, stimulation of calcium transport, and activation of renal 1a-hydroxylase in the kidney. The responses in bone include effects on collagen synthesis; increased alkaline phosphatase, ornithine decarboxylase, citrate decarboxylase, and glucose-6-phosphate dehydrogenase activities; DNA, protein, and phospholipid synthesis; calcium and phosphate transport; and local cytokine/growth factor release. Ultimately, these biochemical events lead to an integrated hormonal response in bone turnover and calcium homeostasis.

B. Measurement of the Efficacy of SMCM Compounds

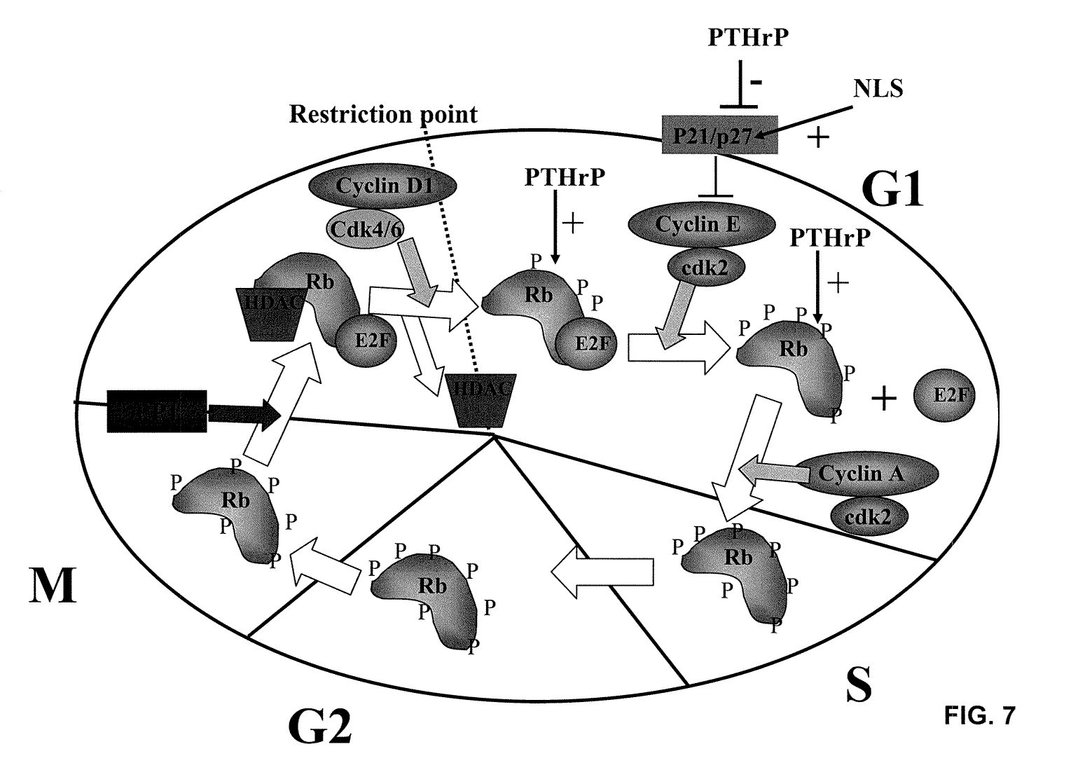

SMCM compounds function as inhibitors of smooth cell activation. The synthesis, selection, and use of SMCM compounds of the present invention, which are capable of modulating smooth muscle activation is within the ability of a person of ordinary skill in the art. For example, well-known in vitro or in vivo assays can be used to determine the efficacy of various candidate SMCM compounds to promote molecular events that modulate smooth muscle cell activation, see, e.g., Lester et al., Endocrine Rev. 10: 420-36 (1989). Further, any in vitro or In vivo assays developed to measure the activity, modification or expression of the molecular markers of cellular activation and proliferation shown in FIG. 7, e.g., cyclin E, cdk2, cyclin A, cyclin D1, and cdk4/6, may be employed to assess the activity of SMCM compounds of the present invention.

The activity of secreted forms of SMCM, e.g., ΔNLS SMCM compounds, may be assessed using in vitro binding assays. For example, osteoblast-like cells which are permanent cell lines with osteoblastic characteristics and possess receptors for PTHrP of either rat or human origin can be used. Suitable osteoblast-like cells include ROS 17/2 (Jouishomme et al., Endocrinology, 130: 53 60 (1992)), UMR 106 (Fujimori et al., Endocrinology, 130: 29 60 (1992)), and the human derived SaOS-2 (Fukuyama et al, Endocrinology, 131: 1757 1769 (1992)). The cell lines are available from American Type Culture Collection, Rockville, Md., and can be maintained in standard specified growth media. Additionally, transfected human embryonic kidney cells (HEK 293) expressing the human PTH1 or PTH2 receptors can also be utilized for in vitro binding assays (Pines et al., Endocrinology, 135: 1713-1716 (1994)). Moreover, A-10 vascular smooth muscle cells express can be utilized for In vitro binding assays of SMCM to PTH/PTHrP receptor (De Miguel et al., Endocrinology 142: 4096-105 (2001)).

For in vitro functional assays, SMCM activities can be tested by contacting a concentration range of the SMCM compound candidate, ΔNLS SMCM compound, with cells in culture in the presence and absence of PTHrP polypeptide, or fragment thereof and assessing the stimulation of the activation of second messenger molecules coupled to the receptors, e.g., the stimulation of cyclic AMP accumulation in the cell or an increase in enzymatic activity of protein kinase C, both of which are readily monitored by conventional assays. See, Jouishomme et al., Endocrinology, 130: 53-60 (1992); Abou-Samra et al., Endocrinology, 125: 2594 2599 (1989); Fujimori et al., Endocrinology, 128: 3032 3039 (1991); Fukayama et al., Endocrinology, 134: 1851 1858 (1994); Abou-Samra et al., Endocrinology, 129: 2547 2554 (1991); and Pines et al., Endocrinology, 135: 1713-1716 (1994). Detailed procedure for handling the cells, setting up the assay, as well as methods for cAMP quantitation, is described in Sistane et al., Pharmacopeial Forum 20: 7509-7520 (1994). Other parameters of PTHrP action include increase In cytosolic calcium and phosphoinositols, p27kip expression, retinoblastoma protein phosphorylation, tritiated thymidine uptake, and alteration in alkaline phosphatase activity. Cell growth can also be monitored as an index of SMCM function.

Immunolocalization of PTHrP mutant compounds can be performed as described by Massfelder et al., Proc. Nat'l Acad. Sci. USA 94: 13630-635 (1997).

As demonstrated in Example 1 and Example 3, cell growth rate, as well as, phosphorylation of molecular markers such as retinoblastoma protein and p27kip1 protein can be monitored in A-10 VSM cells transfected with vectors encoding SMCM compound to assess the effect of overexpression of SMCM polypeptide on cellular activation.

The biological activity, namely the agonist or antagonist properties of SMCM compounds can characterized using any conventional in vivo assays that have been developed to measure the cellular activation of smooth muscle cells. For example, using in vivo assays, candidate SMCM compounds can be characterized by their abilities to inhibit neointimal hyperplasia in rat, pig, or rabbit as described in Example 2, 4, and 5.

### VI. Pharmaceutical Compositions

The SMCM-encoding nucleic acid molecules, SMCM polypeptide compounds, viral carriers of vectors encoding SMCM compounds, and anti-SMCM compound antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, polypeptide, or antibody and a pharmaceutically acceptable carrier. As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition a compound which delays absorption, for example, aluminum monostearate and gelatin.

Sterile injectable solutions can be prepared by incorporating the active compound (e.g., a SMCM compound or anti-SMCM compound antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as aiginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared as pharmaceutical compositions in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsuiated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to Infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect In association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the Invention can be Inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., Proc. Natl. Acad. Sci. USA 91:3054-3057 (1994)). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

### VII. Treatment of Disease and Disorders

A. Prophylactic and Therapeutic Uses of the Compositions of the Invention

The SMCM compounds of the present invention are useful in potential prophylactic and therapeutic applications implicated in a variety of disorders in a subject (See Diseases and Disorders). Diseases and disorders that are characterized by increased (relative to a subject not suffering from the disease or disorder) levels or biological activity of smooth muscle cell activation and proliferation can be treated with SMCM-based therapeutic compounds that antagonize (i.e., reduce or inhibit) activity, which can be administered in a therapeutic or prophylactic manner. Therapeutic compounds that can be utilized include, but are not limited to: (i) an aforementioned SMCM compound, or analogs, derivatives, fragments or homologs thereof; (ii) anti-SMCM compound antibodies to a PTHrP or SMCM compound; (iii) polynucleotide encoding an SMCM compound; (iv) administration of a viral vector containing a vector encoding an SMCM compound; or (v) modulators (i.e., Inhibitors, agonists and antagonists, including additional peptide mimetic of the invention or antibodies specific to a peptide of the invention) that alter the interaction between an aforementioned compound and its binding partner.

Increased or decreased levels can be readily detected by quantifying SMCM compound polypeptide and/or RNA, by obtaining a patient tissue sample (e.g., from biopsy tissue) and assaying it in vitro for RNA or polypeptide levels, structure and/or activity of the expressed polypeptides (or mRNAs of an aforementioned polypeptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g. Northern assays, dot blots, in situ hybridization, and the like).

A cDNA encoding the SMCM compound can be useful in gene therapy, and the polypeptide SMCM compound can be useful when administered to a subject in need thereof. By way of non-limiting example, the compositions of the invention will have efficacy for treatment of patients suffering from the mentioned disorders mentioned in the Diseases and Disorders, infra.

i. Prophylactic Methods

In one aspect, the invention provides a method for preventing a disease or condition associated with smooth muscle cell activation and proliferation in a subject, by administering to the subject an SMCM compound, a polynucleotide encoding an SMCM compound, administration of a viral vector containing a vector encoding an SMCM compound, or SMCM compound mimetic that inhibits smooth muscle cell activation and cellular proliferation.

Subjects at risk for a disease that is caused or contributed to by aberrant smooth muscle cell activation and proliferation can be identified by, for example, any or a combination of diagnostic or prognostic assays as described herein. Administration of a prophylactic SMCM compound can occur prior to the manifestation of symptoms characteristic of the aberrancy, such that a disease or disorder is prevented or, alternatively, delayed in its progression. Depending upon the type of aberrancy, for example, a SMCM compound, SMCM compound mimetic, virus carrying a vector encoding an SMCM compound, or anti-SMCM compound antibody, which acts as an antagonist to smooth muscle cell activation and proliferation, the appropriate compound can be determined based on screening assays described herein.

ii. Therapeutic Methods

Another aspect of the invention includes methods of inhibiting smooth muscle cell activation and proliferation in a subject for therapeutic purposes. The modulatory method of the invention involves contacting a cell with a compound of the present invention, that inhibits smooth muscle cell activation and cell proliferation. A compound that inhibits smooth muscle cell activation and proliferation is described herein, such as a nucleic acid or a polypeptide, an anti-SMCM compound antibody, or a virus containing a vector encoding an SMCM compound. These methods can be performed in vitro (e.g., by culturing the cell with the SMCM compound) or, alternatively, in vivo (e.g., by administering the SMCM compound to a subject). As such, the invention provides methods of treating an individual afflicted with a disease or disorder manifested by aberrant activation of smooth muscle and proliferation. In one embodiment, the method involves administering an SMCM compound (e.g., a compound identified by a screening assay described herein), or combination of SMCM compounds that inhibit smooth muscle cell proliferation and proliferation.

B. Determination of the Biological Effect of the SMCM-Based Therapeutic

In various embodiments of the invention, suitable in vitro or in vivo assays are performed to determine the effect of a specific SMCM-based therapeutic and whether its administration is indicated for treatment of the affected tissue in a subject.

In various specific embodiments, in vitro assays can be performed with representative cells of the type(s) involved in the patient's disorder, to determine if a given SMCM-based therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

C. Diseases and Disorder

Smooth muscle cell proliferation is associated with numerous diseases, all of which could be effected by the development of a smooth muscle cell proliferation-modulating agent. The invention provides for both prophylactic and therapeutic methods of treating a subject at risk of (or susceptible to) a disorder or having a disorder associated with aberrant smooth muscle cell activation, e.g., but not limited to, uterine fibroid tumors, prostatic hypertrophy, bronchial asthma, portal hypertension in cirrhosis, bladder disease, pulmonary and systemic arterial hypertension, atherosclerosis, and vascular restenosis after angioplasty are thought to be the result of smooth muscle cell activation and excessive smooth muscle cell proliferation. PTHrP has been implicated in disorders manifested by smooth muscle cell activation and proliferation, therefore, SMCM compounds are useful in the treatment of smooth muscle cell activation and proliferation mediated by PTHrP expression.

The SMCM compounds of the present invention are useful in the prevention or therapeutic treatment of uterine leiomyomas (fibroids of myomas). Uterine leiomyomas (fibroids or myomas) are benign tumors of the human uterus and develop from uterine smooth muscle cells. M. Yoshida et al., have demonstrated (Endocr J; 46(1):81-90 (1999)) that PTHrP may act as a local cell growth modifier in an autocrine/paracrine fashion on uterine leiomyomas. The SMCM compounds of the present invention are useful in the prevention or therapeutic treatment of prostate cancer and prostatic hyperplasia. Benign prostatic hyperplasia (BPH), one of the most common diseases in elderly men, is characterized by abnormal proliferation of the stromal cells, and SMCs constitute a major cellular component of prostatic stroma (Shapiro E, et al., J Urol 147: 1167-1170 (1992)). Additionally, SMC proliferation and tension play important roles in bladder outflow obstruction secondary to BPH (Tenniswood M P, et al., Cancer Metast Rev 11: 197-220 (1992)). Further, PTHrP is expressed in both prostate cancer and benign prostatic hyperplasia (Asadi F et al., Hum Pathol. 27(12):1319-23 (1996)); additionally, PTHrP increases the growth and enhances the osteolytic effects of prostate cancer cells (Tovar Sepulveda Va., Falzon M. Mol Cell Endocrinol; 204(1-2):51-64 (2003)). The SMCM compounds of the present invention are useful in the prevention or therapeutic treatment of portal hypertension in cirrhosis. In the liver, various cholestatic liver diseases as well as regeneration after submassive necrosis are accompanied by a striking increase in the number of bile ductules. T. Roskams et al., (Histopathology, 23(1):11-9 (1993)) in studying the immunohistochemical expression of PTHrP in various human livers, including three normal biopsies, 11 cases of cholestatic liver disease, six cases of focal nodular hyperplasia and three cases of regenerating liver, found that PTHrP is localized in bile ductuiar cells which indicates a role for this hormone in the growth and/or differentiation of human reactive bile ductules.

The SMCM compounds of the present invention are useful in the prevention or therapeutic treatment of disease of the bladder. PTHrP has been implicated In bladder diseases, including neuropathic bladder. Vaidyanathan S et al. (Spinal Cord; 38(9):546-51 (2000)) demonstrated that the epithelium of non-neuropathic bladder showed no immunostaining, or at the most, very faint positive staining for PTHrP. In contrast, positive immunostaining for PTHrP was observed far more frequently in the vesical epithelium of neuropathic bladder. Vascular medial thickening, a hallmark of hypertension, is associated with vascular smooth muscle cell (VSMC) hypertrophy and hyperplasia (Nolan B P et al., Am J Hypertens.; 16(5 Pt 1):393-400 (2003)).

The SMCM compounds of the present invention are useful in the prevention or therapeutic treatment of bronchial asthma. SM Puddicombe et al., (Am J Respir Cell Mol. Biol. 28(1):61-8 (2003)) have demonstrated that p21(waf) overexpression in asthma influences cell proliferation and survival. SMC proliferation can have a drastic effect on asthma, as longer-term structural changes occurring in the airways of patients with asthma are driven by SMC hyperplasia and hypertrophy (Freyer AM., Am J Respir Cell Mol. Biol.; 25(5):569-76 (2001)).

The SMCM compounds of the present invention are useful in the prevention or therapeutic treatment of pulmonary and arterial hypertension. Pulmonary hypertension can be a rapidly progressive and fatal disease characterized by changes in vascular structure and function associated with smooth muscle cell proliferation and migration Into the neointima, among other things (Rabinovitch, Cardiovasc Res. 34:268-272 (1997); Nichols et al., Endocrinology 119: 349 (1986)).

The SMCM compounds of the present invention are useful in the prevention or therapeutic treatment of atherosclerosis, and vascular restenosis after angioplasty. The proliferation and migration of SMCs have been acknowledged as playing a key role In the pathophysiology of cardiovascular disease (Martinez-Gonzalez J et al., Circ Res.; 92(1):96-103 (2003)), including post-angioplasty restenosis leading to neointima formation (Segev A, et al., Cardiovasc Res; 53(1):232-41 (2002).

### VIII. Screening and Detection Methods

The compounds of the invention can be used to express SMCM compounds (e.g., via a recombinant expression vector in a host cell in gene therapy applications), to detect SMCM mRNA (e.g., in a biological sample) or a genetic lesion in a SMCM gene, and to modulate PTHrP or SMCM compound activity, as described further, below. In addition, the SMCM polypeptides can be used to screen drugs or compounds that modulate the PTHrP or SMCM compound activity or expression as well as to treat disorders characterized by insufficient or excessive production of PTHrP polypeptides or production of PTHrP polypeptide forms that have aberrant activity compared to PTHrP wild-type polypeptide. In addition, the anti-SMCM compound antibodies of the invention can be used to detect and isolate PTHrP or SMCM compounds and modulate their activity. Accordingly, the present invention further includes novel compounds identified by the screening assays described herein and uses thereof for treatments as described, supra.

A. Screening Assays

The invention provides for methods for identifying modulators, i.e., candidate or test compounds or compounds (e.g., peptides, peptidomimetics, small molecules or other drugs) that bind to SMCM compound or PTHRP polypeptides or have a stimulatory or inhibitory effect on, e.g., SMCM compound or PTHRP polypeptide expression or activity (also referred to herein as “screening assays”). The invention also includes compounds identified in the screening assays described herein.

In one embodiment, the invention includes assays for screening candidate or test compounds which bind to or modulate the activity SMCM compound or PTHRP polypeptides or biologically-active portions thereof. The compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

Libraries of chemical and/or biological mixtures, such as fungal, bacterial, or algal extracts, are known in the art and can be screened with any of the assays described as well as those known to skilled artisans. Examples of methods for the synthesis of molecular libraries can be found in the scientific literature, for example in: DeWitt, et al., Proc. Natl. Acad. Sci. USA 90: 6909 (1993); Erb, et al., Proc. Natl. Acad. Sci. USA 91: 11422 (1994); Zuckermann, et al., J. Med. Chem. 37: 2678 (1994); Cho, et al., Science 261: 1303 (1993); Carrell, et al., Angew. Chem. Int. Ed. Engl. 33: 2059 (1994); Carell, et al., Angew. Chem. Int. Ed. Engl. 33: 2061 (1994); and Gallop, et al., J. Med. Chem. 37: 1233 (1994).

Libraries of compounds can be presented in solution (e.g., Houghten, Biotechniques 13: 412-421 (1992)), or on beads (Lam, Nature 354: 82-84 (1991)), on chips (Fodor, Nature 364: 555-556 (1993)), bacteria (Ladner, U.S. Pat. No. 5,223,409), spores (Ladner, U.S. Pat. No. 5,233,409), plasmids (Cull, et al., Proc. Natl. Acad. Sci. USA 89: 1865-1869 (1992)) or on phage (Scott and Smith, Science 249: 386-390 (1990); Devlin, Science 249: 404-406 (1990); Cwirla, et al., Proc. Natl. Acad. Sci. USA 87: 6378-6382 (1990); Felici, J. Mol. Biol. 222: 301-310 (1991); Ladner, U.S. Pat. No. 5,233,409.).

Determining the ability of a compound to modulate the activity of a SMCM polypeptide can be accomplished, for example, by determining the ability of the SMCM compound to bind to or interact with a SMCM compound target molecule. A target molecule is a molecule that a SMCM compound binds to or interacts with, for example, a molecule on the surface of a cell which expresses a SMCM interacting polypeptide, a molecule on the surface of a second cell, a molecule in the extracellular milieu, a molecule associated with the internal surface of a cell membrane, a cytoplasmic molecule, or a molecule in the nucleus. A SMCM compound target molecule can be a non-SMCM compound molecule or a SMCM compound of the invention. In one embodiment, a SMCM compound target molecule is a component of a signal transduction pathway that facilitates transduction of an extracellular signal (e.g., a mechanical signal, or a chemical signal, e.g., a signal generated by binding of a mitogen to a mitogen target molecule, e.g., PTHrP receptor molecule) through the cell membrane and into the cell. The target, for example, can be a second intracellular polypeptide that has catalytic activity or a polypeptide that facilitates the association of downstream signaling molecules with cellular activation and proliferation. The compounds of the present invention either agonize or antagonize such interactions and the resultant biological responses, measured by the assays described.

Determining the ability of the SMCM polypeptide compound to bind to or interact with a SMCM compound target molecule can be accomplished by one of the methods described above for determining direct binding. In one embodiment, determining the ability of the SMCM polypeptide compound to bind to or interact with a SMCM compound target molecule can be accomplished by determining the activity of the target molecule. For example, the activity of the target molecule can be determined by detecting induction of a cellular second messenger of the target (i.e., intracellular Ca2+, diacylglycerol, 1P3, etc.), detecting catalytic/enzymatic activity of the target and appropriate substrate, detecting the induction of a reporter gene (comprising a SMCM-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a SMCM compound or biologically-active portion thereof with a test compound and determining the ability of the test compound to bind to the SMCM polypeptide SMCM compound or biologically-active portion thereof. Binding of the test compound to the SMCM compound can be determined either directly or indirectly as described above. In one such embodiment, the assay comprises contacting the SMCM compound or biologically-active portion thereof with a known compound which binds SMCM to form an assay mixture, contacting the assay mixture with a test compound, and determining the ability of the test compound to interact with a SMCM compound, wherein determining the ability of the test compound to interact with a SMCM compound comprises determining the ability of the test compound to preferentially bind to SMCM or biologically-active portion thereof as compared to the known compound.

In still another embodiment, an assay is a cell-free assay comprising contacting SMCM compound or biologically-active portion thereof with a test compound and determining the ability of the test compound to modulate (e.g. stimulate or inhibit) the activity of the SMCM compound or biologically-active portion thereof. Determining the ability of the test compound to modulate the activity of SMCM can be accomplished, for example, by determining the ability of the SMCM compound to bind to an SMCM compound target molecule by one of the methods described above for determining direct binding. In an alternative embodiment, determining the ability of the test compound to modulate the activity of SMCM compound can be accomplished by determining the ability of the SMCM compound to further modulate a SMCM compound target molecule. For example, the catalytic/enzymatic activity of the target molecule on an appropriate substrate can be determined as described, supra.

In yet another embodiment, the cell-free assay comprises contacting the SMCM compound or biologically-active portion thereof with a known compound which binds SMCM compound to form an assay mixture, contacting the assay mixture with a test compound, and determining the ability of the test compound to interact with a SMCM compound, wherein determining the ability of the test compound to interact with an SMCM compound comprises determining the ability of the SMCM compound to preferentially bind to or modulate the activity of a SMCM compound target molecule.

In more than one embodiment of the above assay methods of the invention, it can be desirable to immobilize either SMCM compound or its target molecule to facilitate separation of complexed from uncomplexed forms of one or both of the polypeptides, as well as to accommodate automation of the assay. Binding of a test compound to SMCM compound, or interaction of SMCM compound with a target molecule in the presence and absence of a candidate compound, can be accomplished in any vessel suitable for containing the reactants. Examples of such vessels include microtiter plates, test tubes, and micro-centrifuge tubes. In one embodiment, a fusion polypeptide can be provided that adds a domain that allows one or both of the polypeptides to be bound to a matrix. For example, GST-SMCM fusion polypeptides or GST-target fusion polypeptides can be adsorbed onto glutathione sepharose beads (Sigma Chemical, St. Louis, Mo.) or glutathione derivatized microtiter plates, that are then combined with the test compound or the test compound and either the non-adsorbed target polypeptide or SMCM compound, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of SMCM compound binding or activity determined using standard techniques.

Other techniques for immobilizing polypeptides on matrices can also be used in the screening assays of the invention. For example, either the SMCM compound or its target molecule can be immobilized utilizing conjugation of biotin and streptavidin. Biotinylated SMCM compound or target molecules can be prepared from biotin-NHS(N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with SMCM compound or target molecules, but which do not interfere with binding of the SMCM compound to its target molecule, can be derivatized to the wells of the plate, and unbound target or SMCM compound trapped in the wells by antibody conjugation. Methods for detecting such complexes, in addition to those described above for the GST-immobilized complexes, include immunodetection of complexes using antibodies reactive with the SMCM compound or target molecule, as well as enzyme-linked assays that rely on detecting an enzymatic activity associated with the SMCM compound or target molecule.

In another embodiment, modulators of SMCM compound expression are identified in a method wherein a cell is contacted with a candidate compound and the expression of SMCM mRNA or polypeptide in the cell is determined. The level of expression of SMCM mRNA or polypeptide in the presence of the candidate compound is compared to the level of expression of SMCM mRNA or polypeptide in the absence of the candidate compound. The candidate compound can then be identified as a modulator of SMCM mRNA or polypeptide expression based upon this comparison. For example, when expression of SMCM mRNA or polypeptide is greater (i.e., statistically significantly greater) in the presence of the candidate compound than in its absence, the candidate compound is identified as a stimulator of SMCM mRNA or polypeptide expression. Alternatively, when expression of SMCM mRNA or polypeptide is less (statistically significantly less) in the presence of the candidate compound than in its absence, the candidate compound is identified as an inhibitor of SMCM mRNA or polypeptide expression. The level of SMCM mRNA or polypeptide expression in the cells can be determined by methods described herein for detecting SMCM mRNA or polypeptide.

In yet another aspect of the invention, the SMCM compounds can be used as “bait polypeptides” in a two-hybrid assay or three hybrid assay (see, e.g., U.S. Pat. No. 5,283,317; Zervos, et al., Cell 72: 223-232 (1993); Madura, et al., J. Biol. Chem. 268: 12046-12054 (1993); Bartel, et al., Biotechniques 14: 920-924 (1993); Iwabuchi, et al., Oncogene 8: 1693-1696 (1993); and Brent WO 94/10300), to identify other molecules, e.g., polypeptides, that bind to or interact with SMCM (“SMCM-binding molecules” or “SMCM-bp”) and modulate SMCM activity. Such SMCM-binding molecules are also likely to be involved in the propagation of signals by the SMCM compounds as, for example, upstream or downstream elements of a the SMCM pathway.

The two-hybrid system is based on the modular nature of most transcription factors, which consist of separable DNA-binding and activation domains. Briefly, the assay utilizes two different DNA constructs. In one construct, the gene that codes for SMCM compound is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. If the “bait” and the “prey” polypeptides are able to interact, in vivo, forming a SMCM-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the polypeptide which interacts with SMCM compound.

In still another embodiment, a system comprising structural information relating to the SMCM compound atomic coordinates can be obtained by biophysical techniques, e.g., x-ray diffraction. Binding between a SMCM compound and a compound can be assessed by x-ray diffraction to determine the x-ray crystal structure of the SMCM compound complexes, e.g., target polypeptide/drug complex. Alternatively; NMR may be used to analyze the change in chemical shifts observed after a compound binds with the SMCM compound. Such approaches may be used to screen for compounds based on their binding interaction with SMCM compound.

The invention further pertains to SMCM compounds identified by the aforementioned screening assays and uses thereof for treatments as described herein.

B. Detection Assays

i. Detection of SMCM Expression

An exemplary method for detecting the presence or absence of SMCM compound in a biological sample involves obtaining a biological sample from a test subject and contacting the biological sample with a compound or a compound capable of detecting SMCM compound or nucleic acid (e.g., mRNA, genomic DNA) that encodes SMCM compound such that the presence of SMCM compound is detected in the biological sample. A compound for detecting SMCM mRNA or genomic DNA is a labeled nucleic acid probe capable of hybridizing to SMCM mRNA or genomic DNA. The nucleic acid probe can be, for example, a full-length SMCM nucleic acid or a portion thereof, such as an oligonucleotide of at least 5, 15, 30, 50, 100, 250 or 500 nucleotides in length and sufficient to specifically hybridize under stringent conditions to SMCM mRNA or genomic DNA. Other suitable probes for use in the diagnostic assays of the invention are described herein.

An example of a compound for detecting a SMCM compound is an antibody raised against SMCM compound, capable of binding to the SMCM compound, preferably an antibody with a detectable label. Antibodies can be polyclonal, or more preferably, monoclonal. An intact antibody, or a fragment thereof (e.g. Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another compound that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect SMCM mRNA, polypeptide, or genomic DNA in a biological sample in vitro as well as in vivo. For example, in vitro techniques for detection of SMCM mRNA include Northern hybridizations and in situ hybridizations. In vitro techniques for detection of SMCM compound include enzyme linked immunosorbent assays (ELISAs), Western blots, immunoprecipitations, and immunofluorescence. In vitro techniques for detection of SMCM genomic DNA include Southern hybridizations. Furthermore, in vivo techniques for detection of SMCM compound include introducing into a subject a lebeled anti-SMCM antibody. For example, the antibody can be labeled with a radioactive marker whose presence and location in a subject can be detected by standard imaging techniques. In one embodiment, the biological sample contains polypeptide molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or compound capable of detecting SMCM compound, mRNA, or genomic DNA, such that the presence of SMCM compound, mRNA or genomic DNA is detected in the biological sample, and comparing the presence of SMCM compound, mRNA or genomic DNA in the control sample with the presence of SMCM compound, mRNA or genomic DNA in the test sample.

The invention also encompasses kits for detecting the presence of SMCM compound in a biological sample as well as instructions for its use. For example, the kit can comprise: a labeled compound or compound capable of detecting SMCM compound or mRNA in a biological sample; means for determining the amount of SMCM compound in the sample; and means for comparing the amount of SMCM compound in the sample with a standard. The compound or compound can be packaged in a suitable container. The kit can further comprise instructions for using the kit to detect SMCM compound or nucleic acid.

### IX. SMCM Compound Gene Therapy

The present invention also provides for a SMCM compound encoding nucleic acid molecule linked to a vector. The vector may be a self-replicating vector or a replicative incompetent vector. The vector may be a pharmaceutically acceptable vector for methods of gene therapy. An example of replication incompetent vector is LNL6 (Miller, A. D. et al., BioTechniques 7: 980-990 (1989))

The invention features expression vectors for in vivo transfection and expression in particular cell types of SMCM compounds antagonize smooth muscle cell activation and proliferation.

Expression constructs of SMCM compound may be administered in any biologically effective carrier that is capable of effectively delivering a polynucleotide sequence encoding the SMCM compound to cells in vivo. Approaches include insertion of the subject gene in viral vectors including recombinant retroviruses, baculovirus, adenovirus, adeno-associated virus and herpes simplex virus-1, or recombinant bacterial or eukaryotic plasmids. Viral vectors transfect cells directly, plasmid DNA can be delivered with the help of, for example, cationic liposomes or derivatized (e.g., antibody conjugated) polylysine conjugates, gramacidin S, artificial viral envelopes or other such intracellular carriers, as well as direct injection of the gene construct or CaPO₄ precipitation carried out in vivo.

Any of the methods known in the art for the insertion of polynucleotide sequences into a vector may be used. See, for example, Sambrook et al., Molecular Cloning: A Laboratory Manual, Cold Spring Harbor Laboratory, Cold Spring Harbor, N.Y. (1989) and Ausubel et al., Current Protocols in Molecular Biology, J. Wiley & Sons, N.Y. (1992), both of which are incorporated herein by reference. Conventional vectors consist of appropriate transcriptional/translational control signals operatively linked to the polynucleotide sequence for a particular SMCM compound encoding polynucleotide sequence. Promoters/enhancers may also be used to control expression of SMCM compound. Promoter activation may be tissue specific or inducible by a metabolic product or administered substance. Such promoters/enhancers include, but are not limited to, the native E2F promoter, the cytomegalovirus immediate-early promoter/enhancer (Karasuyama et al., J. Exp. Med., 169: 13 (1989)); the human beta-actin promoter (Gunning et al., Proc. Natl. Acad. Sci. USA, 84: 4831 (1987); the glucocorticoid-inducible promoter present in the mouse mammary tumor virus long terminal repeat (MMTV LTR) (Klessig et al., Mol. Cell. Biol., 4: 1354 (1984)); the long terminal repeat sequences of Moloney murine leukemia virus (MuLV LTR) (Weiss et al., RNA Tumor Viruses, Cold Spring Harbor Laboratory, Cold Spring Harbor, N.Y. (1985)); the SV40 early region promoter (Bernoist and Chambon, Nature, 290:304 (1981)); the promoter of the Rous sarcoma virus (RSV) (Yamamoto et al., Cell, 22:787 (1980)); the herpes simplex virus (HSV) thymidine kinase promoter (Wagner et al., Proc. Natl. Acad. Sci. USA, 78: 1441 (1981)); the adenovirus promoter (Yamada et al., Proc. Natl. Acad. Sci. USA, 82: 3567 (1985)).

Expression vectors compatible with mammalian host cells for use in gene therapy of tumor cells include, for example, plasmids; avian, murine and human retroviral vectors; adenovirus vectors; herpes viral vectors; and non-replicative pox viruses. In particular, replication-defective recombinant viruses can be generated in packaging cell lines that produce only replication-defective viruses. See Current Protocols in Molecular Biology: Sections 9.10-9.14 (Ausubel et al., eds.), Greene Publishing Associates, 1989.

Specific viral vectors for use in gene transfer systems are now well established. See For example: Madzak et al., J. Gen. Virol., 73: 1533-36 (1992: papovavirus SV40); Berkner et al., Curr. Top. Microbiol. Immunol., 158: 39-61 (1992: adenovirus); Moss et al., Curr. Top. Microbiol. Immunol., 158: 25-38 (1992: vaccinia virus); Muzyczka, Curr. Top. Microbiol. Immunol., 158: 97-123 (1992: adeno-associated virus); Margulskee, Curr. Top. Microbiol. Immunol., 158: 67-93 (1992: herpes simplex virus (HSV) and Epstein-Barr virus (HBV)); Miller, Curr. Top. Microbiol. Immunol., 158: 1-24 (1992: retrovirus); Brandyopadhyay et al., Mol. Cell. Biol., 4: 749-754 (1984: retrovirus); Miller et al., Nature, 357: 455-450 (1992: retrovirus); Anderson, Science, 256: 808-813 (1992: retrovirus), all of which are incorporated herein by reference.

Several methods of transferring potentially therapeutic genes to defined cell populations are known. See, e.g., Mulligan, Science, 260: 920-31 (1993). These methods include: (1) Direct gene transfer (see, e.g., Wolff et al., Science 247: 1465-68 (1990)); (2) Jposome-mediated DNA transfer (see, e.g., Caplen et al., Nature Med., 3: 39-46 (1995); Crystal, Nature Med., 1: 16-17 (1995); Gao and Huang, Biochem. Biophys. Res. Comm., 179: 280-85 (1991)); (3) Retrovirus-mediated DNA transfer (see, e.g., Kay et al., Science, 262: 117-19 (1993); Anderson, Science, 256: 808-13 (1992)); (4) DNA Virus-mediated DNA transfer with viruses including adenoviruses (preferably Ad-2 or Ad-0 based vectors), herpes viruses (preferably herpes simplex virus based vectors), baculoviruses, and parvoviruses (preferably “defective” or non-autonomous parvovirus based vectors, more preferably adeno-associated virus based vectors, most preferably AAV-2 based vectors) (see, e.g. Ali, et al., Gene Therapy 1: 367-84 (1994); U.S. Pat. No. 4,797,368, incorporated herein by reference, and U.S. Pat. No. 5,139,941, incorporated herein by reference).

The choice of a particular vector system for transferring the gene of interest will depend on a variety of factors. One important factor is the nature of the target cell population. Retroviral vectors have been extensively studied and used in a number of gene therapy applications.

X. Use of SMCM Compositions as Coatings for Devices

The present invention also provides stents and catheters, comprising a generally tubular structure (which includes for example, spiral shapes), the surface of which is coated with a composition described above. A stent is a scaffolding, usually cylindrical in shape, that may be inserted into a body passageway (e.g., bile ducts) or a portion of a body passageway, which has been narrowed, irregularly contoured, obstructed, or occluded by a disease process (e.g., ingrowth by a tumor) in order to prevent closure or reclosure of the passageway. Stents act by physically holding open the walls of the body passage into which they are inserted.

Commercially available poly(ethylene oxide) [PEO] and poly (acrylic acid) [PAA] gel-coated balloon angioplasty catheters can be used investigated for their use as local drug delivery systems in terms of gel/solute interactions, solute loading, and release kinetics (Gehrke et al., in Intelligent Materials & Novel Concepts for Controlled Release Technologies, S. Dinh and J. DeNuzzio, Eds., ACS Symposium Series, Washington, D.C., 728, 43-53 (1999)). Loading of proteins in PEO-gel coatings can be approximately doubled with the addition of soluble dextran to the loading solution. Release of solutes, e.g., SMCM compound or virus carrying polynucleotides encoding SMCM compound, from gel coatings is diffusion limited, though resistance may be due to the boundary layer as well as the gel.

A variety of stents and catheters may be utilized within the context of the present invention, including, for example, esophageal stents, vascular stents, biliary stents, pancreatic stents, ureteric and urethral stents, lacrimal stents, Eustachiana tube stents, fallopian tube stents and tracheal/bronchial stents, vascular catheters, and urethral catheters.

Stents and catheters may be readily obtained from commercial sources, or constructed in accordance with well-known techniques. Representative examples of stents include those described in U.S. Pat. No. 4,768,523, entitled “Hydrogel Adhesive,” U.S. Pat. No. 4,776,337, entitled “Expandable Intraluminal Graft, and Method and Apparatus for Implanting and Expandable Intraluminal Graft;” U.S. Pat. No. 5,041,126 entitled “Endovascular Stent and Delivery System;” U.S. Pat. No. 5,052,998 entitled “Indwelling Stent and Method of Use,” U.S. Pat. No. 5,064,435 entitled “Self-Expanding Prosthesis Having Stable Axial Length;” U.S. Pat. No. 5,089,606, entitled “Water-=insoluble Polysaccharide Hydrogel Foam for Medical Applications;” U.S. Pat. No. 5,147,370, entitled “Nitinol Stent for Hollow Body Conduits;” U.S. Pat. No. 5,176,626, entitled “Indwelling Stent;” U.S. Pat. No. 5,213,580, entitled “Biodegradable polymeric Endoluminal Sealing Process.”

Stents and catheters may be coated with SMCM compound compositions, or polynucleotide encoding an SMCM compound, or virus containing a vector encoding an SMCM compound, in a variety of manners, including for example: (a) by directly affixing to the device an SMCM compound (e.g., by either spraying the stent with a polymer/drug film, or by dipping the stent into a polymer/drug solution), (b) by coating the device with a substance such as a hydrogel which will in turn absorb the SMCM compound, (c) by interweaving SMCM compound coated thread (or the polymer itself formed into a thread) into the device structure, (d) by inserting the device into a sleeve or mesh which is comprised of or coated with an SMCM compound, or (e) constructing the device itself with an SMCM compound. Within preferred embodiments of the invention, the composition should firmly adhere to the device during storage and at the time of insertion The SMCM compound should also preferably not degrade during storage, prior to insertion, or when warmed to body temperature after expansion inside the body. In addition, it should preferably coat the device smoothly and evenly, with a uniform distribution of SMCM compound, while not changing the device contour. Within preferred embodiments of the invention, the release of the SMCM compound should be uniform, predictable, and may be prolonged into the tissue surrounding the device once it has been deployed. For vascular stents and catheters, in addition to the above properties, the SMCM compound composition should not render the stent or catheter thrombogenic (causing blood clots to form), or cause significant turbulence in blood flow (more than the stent itself would be expected to cause if it was uncoated).

Patches may also be prepared from materials that contain SMCM compounds or polynucleotides encoding SMCM compounds, with or without a viral earner. For example, patch materials, e.g., but not limited to, Gelfoam or Polyvinyl alcohol (PVA), or other suitable material, may be used. Such patches may be used prophylactically or therapeutically to deliver SMCM compound or polynucleotide encoding SMCM compound when contacted with a cell.

### XI. Systems and Methods for Structure-Based Rational Drug Design

The SMCM compounds described above antagonize the cellular activation and excessive proliferation of smooth muscle cells. Methods of structure-based drug design using crystalline polypeptides are described in at least U.S. Pat. Nos. 6,329,184 and 6,403,330 both to Uppenbergi. Methods for using x-ray topography and diffractometry to improve protein crystal growth are described in U.S. Pat. No. 6,468,346 (Arnowitz, et al.). Methods and apparatus for automatically selecting Bragg reflections and systems for automatically determining crystallographic orientation are described in U.S. Pat. No. 6,198,796 (Yokoyama, et al.). Methods for the preparation and labeling of proteins for NMR with ¹³C, ¹⁵N, and ²H for structural determinations are described in U.S. Pat. No. 6,376,253 (Anderson, et al.). NMR spectroscopy of large or complex proteins is described in U.S. Pat. No. 6,198,281 (Wand, et al.). Use of nuclear magnetic resonance to design ligands to target biomolecules is described in U.S. Pat. No. 5,989,827 (Fesik, et al.). The process of rational drug design of SMCM compounds with nuclear magnetic resonance includes the steps of: (a) identifying a candidate SMCM compound that is a potential ligand to the target molecule (such as a PTHrP receptor) using two-dimensional ¹⁵N/¹H NMR correlation spectroscopy; (b) forming a binary complex by binding the candidate SMCM compound to the target molecule, and (c) determining the three dimensional structure of the binary complex and thus the spatial orientation of the candidate SMCM compound on the target molecule. The process of rational drug design of bone morphogenetic protein mimetics with x-ray crystallography is accomplished in a similar manner, but structural data is first obtained by forming crystals of the candidate SMCM compound that is a potential ligand to the target molecule (or co-crystals of the complex), and obtaining a data set of the atomic reflections after x-ray irradiation. These techniques are known to those skilled in the art in view of the teachings provided herein.

Refinements to the candidate SMCM compound are then made to increase the affinity of the candidate SMCM compound for the target molecule. Refinements include constraining and cyclizing the SMCM compound or incorporation of non-classical amino acids that induce conformational constraints. A constrained, cyclic or rigidized SMCM compound may be prepared synthetically, provided that in at least two positions in the sequence of the SMCM compound, an amino acid or amino acid analog is inserted that provides a chemical functional group capable of crosslinking to constrain, cyclize or rigidize the SMCM compound after treatment to form the crosslink. Cyclization will be favored when a turn-inducing amino acid is incorporated. Examples of amino acids capable of crosslinking a SMCM compound are cysteine to form disulfides, aspartic acid to form a lactone or a lactam, and a chelator such as gamma-carboxyl-glutamic acid (Gla) (Bachem) to chelate a transition metal and form a cross-link. Protected gamma-carboxyl glutamic acid may be prepared by modifying the synthesis described by Zee-Cheng and Olson (Biophys. Biochem. Res. Commun., 94:1128-1132 (1980)). A SMCM compound in which the peptide sequence comprises at least two amino acids capable of crosslinking may be treated, e.g., by oxidation of cysteine residues to form a disulfide or addition of a meal ion to form a chelate, so as to crosslink the peptide and form a constrained, cyclic or rigidized SMCM compound.

The present invention provides strategies to systematically prepare cross-links. For example, if four cysteine residues are incorporated In the peptide sequence, different protecting groups may be used (see, Hiskey, in The Peptides: Analysis, Synthesis, Biology, Vol. 3, Gross and Meienhofer, eds., Academic Press: New York, pp. 137-167 (1981); Ponsanti et al., Tetrahedron, 46:8255-8266 (1990)). The first pair of cysteines may be deprotected and oxidized, then the second set may be deprotected and oxidized. In this way a defined set of disulfide cross-links may be formed. Alternatively, a pair of cysteines and a pair of chelating amino acid analogs may be incorporated so mat the cross-links are or a different chemical nature.

Non-classical amino acids may be incorporated in the SMCM compound in order to introduce particular conformational motifs, for example but not limited to 1,2,3,4-tetrahydroisoquinoline-3-carboxylate (Kazmierski et al., J. Am. Chem. Soc, 113:2275-2283 (1991)); (2S,3S)-methyl-phenylalanine, (2S,3R)-methyl-phenylalanine, (2R,3S)-methyl-phenylaIanlne and (2R,3R)-methyl-phenylalanine (Kazmierski and Hruby, Tetrahedron Lett. (1991)); 2-aminotetrahydronaphthalene-2-carboxylic acid (Landis, Ph.D. Thesis, University of Arizona (1989)); hydroxy-1,2,3,4-tetrahydroisoquinoline-3-carboxylate (Miyake et al., J. Takeda Res. Labs, 43:53-76 (1989)); beta-carboline (D and L) (Kazmierski, Ph.D. Thesis, University of Arizona (1988)); HIC (histidine isoquinoline carboxylic acid) (Zechel et al., Int. J. Pep. Protein Res., 43 (1991)); and HIC (histidine cyclic urea). Amino acid analogs and peptidomimetics may be Incorporated Into a peptide to induce or favor specific secondary structures, including but not limited to: LL-Acp-(LL-3-amino-2-propenidone-6-carboxylic acid), a beta-turn inducing dipeptide analog (Kemp et al., J. Org. Chem. 50:5834-5838 (1985)); beta-sheet inducing analogs (Kemp et al., Tetrahedron Lett. 29:5081-5082 (1988)); beta-turn including analogs (Kemp et al., Tetrahedron Lett., 29:5057-5060 (1988)); helix inducing analogs (Kemp et al., Tetrahedron Lett., 29:4935-4938 (1988)); gamma-turn inducing analogs (Kemp et al., J. Org. Chem. 54:109:115 (1989)); and analogs provided by the following references: Nagai and Sato, Tetrahedron Lett., 26:647; 14 650 (1985); DiMaio et al., J. Chem. Soc. Perkin Trans, p. 1687 (1989); also a Gly-Ala turn analog (Kahn et al. Tetrahedron Lett., 30:2317 (1989)); amide bond isoetere (Jones et al., Tetrahedron Lett., 29:3853-3856 (1988)) tretazol (Zabrocki et al., J. Am. Chem. Soc. 110:5875-5880 (1988)); DTC (Samanen et al., Int. J. Protein Pep. Res., 35:501:509 (1990)); and analogs taught in Olson et al., J. Am. Chem. Sci., 112:323-333 (1990) and Garvey et al., J. Org. Chem., 56:436 (1990). Conformationally restricted mimetics of beta turns and beta bulges, and peptides containing them, are described in U.S. Pat. No. 5,440,013, issued Aug. 8, 1995 to Kahn.

Once the three-dimensional structure of a SMCM compound (or a refinement of the same) is determined, its therapeutic potential (as an antagonist or agonist) can be examined through the use of computer modeling using a docking program such as GRAM, DOCK, or AUTODOCK. Computer programs that can be used to aid in solving the three-dimensional structure of the SMCM compound and binding complexes thereof include QUANTA, CHARMM, INSIGHT, SYBYL, MACROMODE, and ICM, MOLMOL, RASMOL, AND GRASP (Krauiis, J. Appl. Crystallogr. 24:946-950 (1991)). Most if not all of these programs and others as well can be also obtained from the World Wide Web through the Internet. The rational design of SMCM compounds can include computer fitting of potential agents to the SMCM compound to ascertain how well the shape and the chemical structure of the modified SMCM compound will complement or interfere with the interaction between the SMCM compound and its ligand. Computer programs can also be employed to estimate the attraction, repulsion, and steric hindrance of the potential therapeutic SMCM compound to the SMCM compound target molecule, e.g., PTHrP binding site. Generally, the tighter the fit (e.g., the lower the steric hindrance, and/or the greater the attractive force), the more potent the potential therapeutic SMCM compound will be, since these properties are consistent with a tighter binding constraint. Furthermore, the more specificity in the design of the SMCM compound, the more likely it will not interfere with related SMCM target molecules. This will minimize potential side-effects due to unwanted interactions with other targets

Initially a potential therapeutic SMCM compound can be obtained by screening a random peptide library produced by recombinant bacteriophage for example, (Scott and Smith, Science, 249:386-390 (1990); Cwirla et al., Proc. Natl. Acad. Sci., 87:6378-6382 (1990); Devlin et al, Science, 249:404-406 (1990)) or a chemical library. A candidate therapeutic SMCM compound selected in this manner is then systematically modified by computer modeling programs until one or more promising potential therapeutic SMCM compounds are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)).

Such computer modeling allows the selection of a finite number of rational chemical modifications, as opposed to the countless number of essentially random chemical modifications that could be made, any of which any one might lead to a useful drug. Each chemical modification requires additional chemical steps, which while being reasonable for the synthesis of a finite number of compounds, quickly becomes overwhelming if all possible modifications needed to be synthesized. Thus, through the use of the three-dimensional structural analysis disclosed herein and computer modeling, a large number of these candidate SMCM compounds can be rapidly screened, and a few likely candidate therapeutic SMCM compounds can be determined without the laborious synthesis of untold numbers of SMCM compounds.

The candidate therapeutic SMCM compounds can then be tested in any standard binding assay (including in high throughput binding assays) for its ability to bind to a SMCM compound target or fragment thereof. Alternatively the potential drug can be tested for its ability to modulate (either inhibit or stimulate) the biological activity of a SMCM compound, PTHrP, or another mitogenic compound/stimulus. When a suitable potential drug is identified, a second structural analysis can optionally be performed on the binding complex formed between the ligand and the candidate therapeutic SMCM compound. For all of the screening assays described herein, further refinements to the structure of the candidate SMCM therapeutic compound will generally be necessary and can be made by the successive iterations of any and/or all of the steps provided by the particular drug screening assay, including further structural analysis by x-ray crystallography or NMR, for example.

### EXAMPLES

The following examples are intended to be non-limiting illustrations of certain embodiments of the present invention. All references cited are hereby incorporated herein by reference in their entireties.

### Example 1

### Ser119, Ser130, Thr132 and Ser138 in the Carboxy-Terminus of PTHrP are Required for Activation of VSM Cell Proliferation

### I. General

In earlier studies, the present inventors had demonstrated that while the NLS is required for nuclear targeting, it alone is not sufficient to stimulate proliferation. This requires the carboxy-terminus region of PTHrP, with crude mapping defining the PTHrP(107-139) region as important (Massfelder et al., Proc Natl Acad Sci USA 94(25): 13630 (1997); de Miguel et al., Endocrinology 142(9): 4096 (2001)). Thus, PTHrP(88-139), including the NLS and the carboxy-terminus, is all that is required for stimulating VSM cell proliferation. The purpose of these studies was to more finely, map the carboxy-terminal region.

### II. Methods

A. Construction of PTHrP Mutants

i. PTHrP Deletion Mutants

The PTHrP deletion constructs were generated by as described previously by Massfelder and coworkers (Massfelder et al., Proc. Natl. Acad. Sci. USA 94: 13630-35 (1997)) using the cDNA for human PTHrP(−36/+139) cloned into plasmid pGEM-3 as an initial template. All of the constructs begin with a codon encoding methionine to allow translation. Each has an epitope tag at the C-terminus corresponding to human influenza hemagglutinin (HA) for immunocytochemical detection. Each contains the 3′ untranslated region (UTR) of human β-globin for stabilization of the mRNA (to replace the native PTHrP 3′-UTR AUUUA instability motif that accelerates mRNA degradation) and to provide transcriptional termination, polyadenylation, and splicing signals. Confirmation of the sequences was accomplished by DNA sequencing. The constructs were then subcloned in the pLJ vector (Massfelder et al., Proc. Natl. Acad. Sci. USA, 94: 13630-635 (1997)) and transfected into A-10 cells as described below.

ii. Alanine Mutants

The constructs shown in FIG. 1 were generated by in vitro site-directed mutagenesis as described previously by De Miguel and coworkers (De Miguel et al., Endocrinology, 142: 4096-105 (2001)), using the cDNA for human PTHrP (−36/+139) cloned into plasmid pcDNA-3+ as initial template. Each contains the 3′UTR of human β-globin for stabilization of the mRNA (to replace the native PTHrP 3′UTR AUUUA instability motif which accelerates mRNA degradation) and to provide transcriptional termination, polyadenylation and splicing signals. The constructs also contain a hemagglutinin (HA) tag, not employed In the current study, but previously demonstrated to have no effect on the localization or functional effects of PTHrP in A-10 cells (De Miguel et al., Endocrinology, 142: 4096-105 (2001)). Confirmation of the sequences was accomplished by DNA sequencing.

B. In Vitro Transcription and Translation

To assess the in vitro transcription and translation efficiency of the different mutants of PTHrP, 1 μg of each construct in pGEM-3 plasmid was transcribed and translated in a transcription- and translation-coupled rabbit reticulocyte lysate system (Promega Corp., Madison, Wis.) according to the manufacturer's instructions. Translation products, labeled with [³H]lysine, were analyzed by SDS-PAGE in A-10 to 20% polyacrylamide Tris-glycine gel and then examined using autoradiography.

C. Cell Culture, Stable Transfections, and Cell Counting

The VSM cell line A-10, derived from embryonic rat thoracic aorta, was purchased from the American Type Culture Collection (Rockville, Md.). Cells were cultured in DMEM containing 4.5 g/liter glucose, 10% FBS, 100 U/ml penicillin, 100 μg/ml streptomycin, and 2 mM L-glutamine. Twenty-four hours before transfection, A-10 cells were plated in six-well plates at a density of 1.5×105/well. Transfections were carried out in serum-free medium with 1 μg of each plasmid and 10 μl of lipofectamine (Life Technologies, Inc., Gaithersburg, Md.) for 6 h at 37° C. For transient transfections, after 24 h of recovery cells were replated on glass chamber slides (LabTek, Nalge Nunc International, Naperville, Ill.) and immunostained 48 h later (see below). Stably transfected clones were selected by treatment with 250 μg/ml geneticin (G418, Life Technologies, Inc.). Five to 12 individual clones for each construct were selected, expanded, and analyzed for PTHrP construct expression as described below. Clones were grown continuously in the presence of 250 μg/ml G418. Generally, each growth curve was performed three to four times on each of the clones derived from each construct, for a total of seven to twelve growth curves per construct. While this method assesses the combined effects of PTHrP on cellular proliferation and cell survival, the effects of PTHrP in this system reflect primarily proliferation as determined using tritiated thymidine incorporation (Massfelder et al., Proc. National Acad. Sci. USA, 94: 13630-635 (1997)) and flow cytometry.

D. PTHrP Immunoradiometric Assay

PTHrP secreted from A-10 vascular smooth muscle cells stably transfected with the different PTHrP constructs or infected by the different adenovirus was measured in 24 h conditioned medium obtained at confluence using a two-site immunoradiometric assay (IRMA) specific for PTHrP(1-36) (Massfelder et al., Proc. Natl. Acad. Sci. USA, 94: 13630-635 (1997); De Miguel et al., Endocrinology, 142: 4096-105 (2001)). The detection limit of the assay is 0.5 pM. For measurement of PTHrP in cell extracts, cells were plated in 100 mm culture plates. At confluence, cells were washed with PBS at room temperature and were then resuspended on ice in PBS containing 1% Igepal CA-630 (Sigma, St. Louis, Mo.), 0.5% sodium deoxycholate, 0.1% SDS, 100 μgml aprotinin, and 1 mM sodium orthovanadate. They were sonicated 10 times for 1 sec, incubated on ice for 60 min and then centrifuged at 10,000×g for 10 min at 4° C. The supernatant representing the cell extract was assayed for PTHrP immunoreactivity using the PTHrP (1-36) IRMA described above. Protein was measured according to the method of Bradford, and results are expressed as pmol/mg extract protein.

E. Statistics

Statistical analysis for the growth curves was performed using one-way analysis of variance with the Student-Newman-Keuls modification. All values are expressed as means ±SEM. “P” values less than or equal to 0.05 were considered significant.

### III. Mapping of the PTHrP Carboxy-Terminus Region Using PTHrP Mutants

Deletion of segments composed of amino acids (107-111), (112-120), (121-130) and (131-139) were prepared (FIG. 1) and stably transfected into the rat arterial smooth muscle line, A-10. The (107-111) region was selected for deletion because it is extremely highly conserved among mammalian species, in contrast to the (112-139) region that is less well conserved.

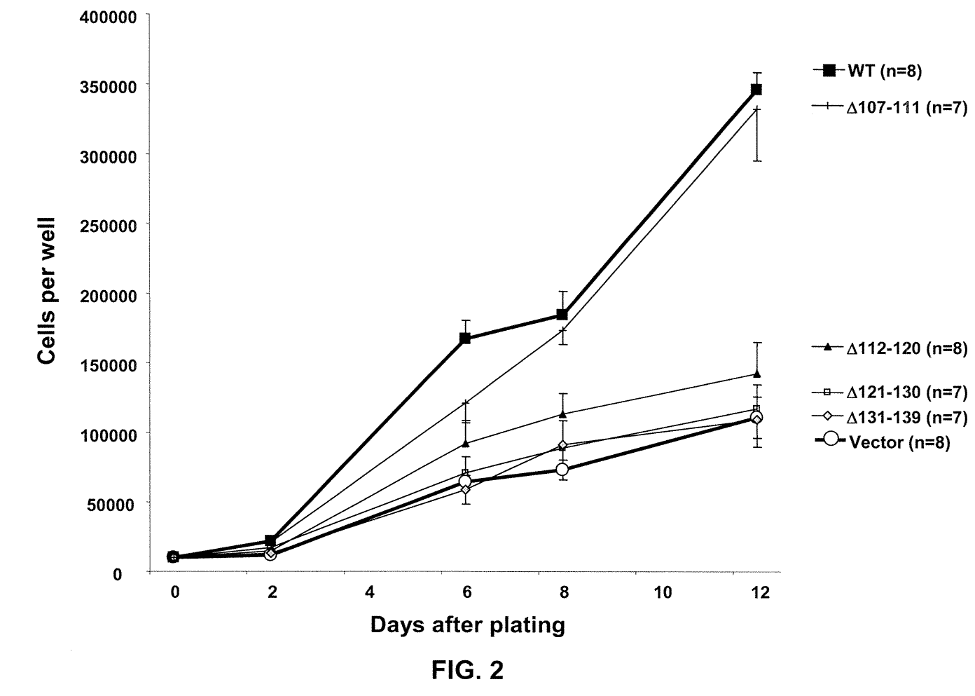

As reported previously (Massfelder et al., Proc Natl Acad Sci U S A., 94: 13630-635 (1997); de Miguel et al., Endocrinology, 142: 4096-105 (2001)), overexpression of wild-type PTHrP (WT) stimulates A-10 cell growth as compared to vector alone-transfected cells (FIG. 2). Surprisingly, as shown in FIG. 2, despite its intense evolutionary conservation, deletion of the (107-111) region had no adverse effect on PTHrP-mediated stimulation of VSM cell proliferation compared with the PTHrP-mediated stimulation observed in cells stably transfected with the wild-type PTHrP construct, i.e., the WT positive experimental control. It was equally surprising that each of the other three deletion mutants, e.g., Δ112-120, Δ121-130, and Δ131-139, essentially completely prevented the PTHrP-mediated stimulation of VSM cell proliferation (FIG. 2).

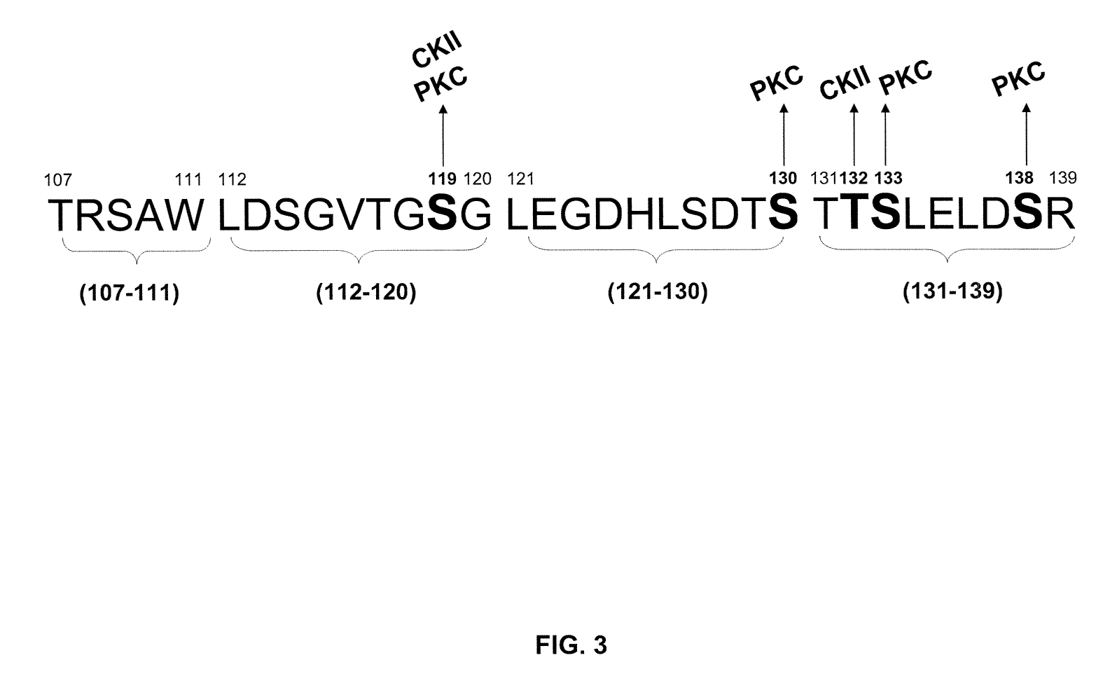

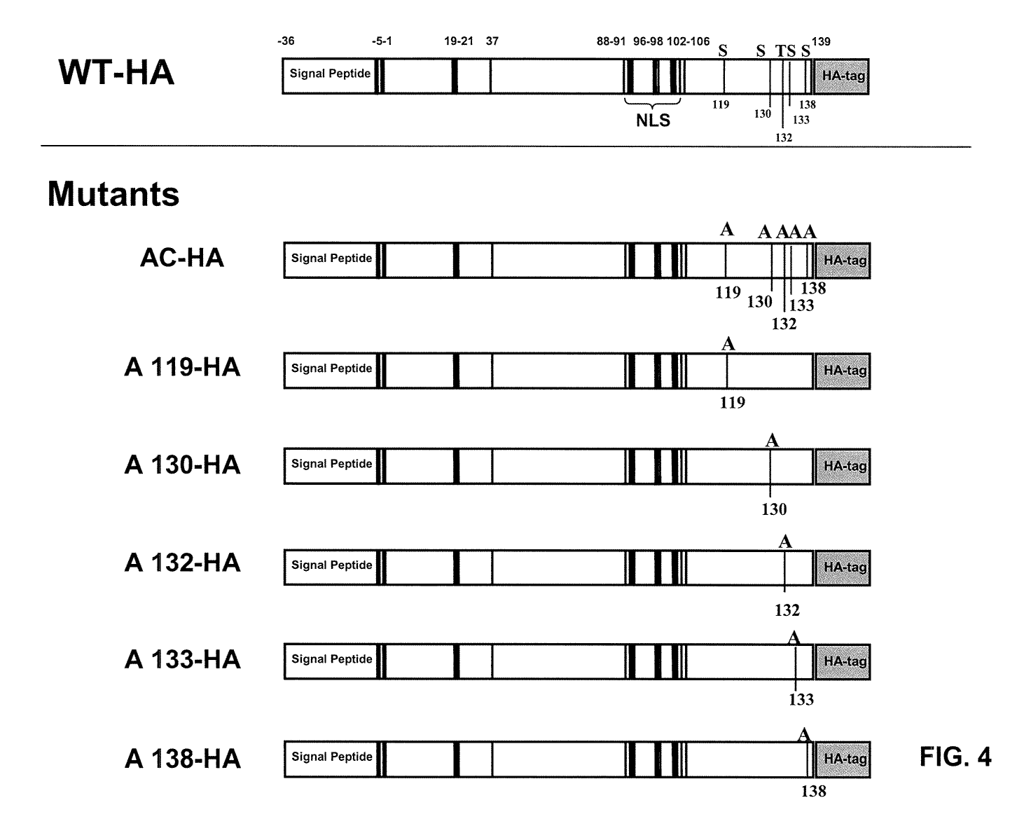

Analysis of the PTHrP(112-139) carboxy-terminus region using the NetPhos 2.0 database indicated that Ser119, Ser130, Thr132, Ser133, and Ser138 could potentially serve as phosphorylation substrates for calmodulin kinase II and/or protein kinase C (FIG. 3). Accordingly, alanine substitution mutants at each of these sites were prepared individually, along with a sixth construct in which all of these serines and threonines were mutated to alanine, and stably transfected Into A-10 cells (FIG. 4).

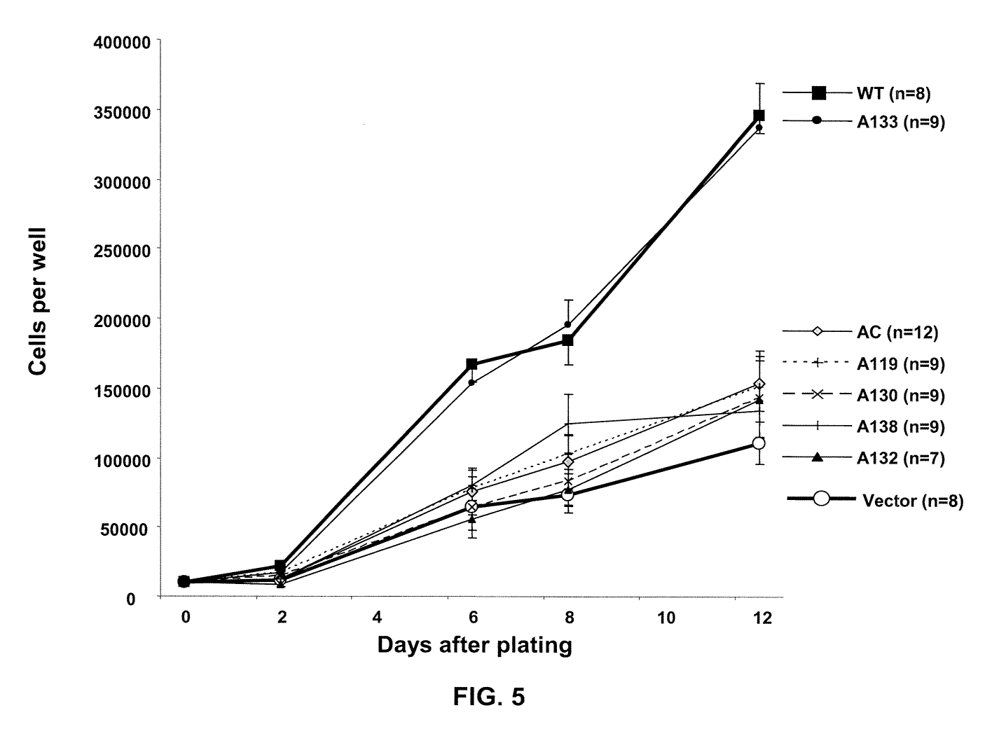

As shown in FIG. 5, Ser133 is not required for the stimulation of VSM cell proliferation by PTHrP. That is, conversion of Ser133 to alanine had no adverse effect on proliferation, with these cells growing as rapidly as A-10 cells overexpressing wild-type PTHrP, and faster than vector-alone transfected cells. In contrast, each of the other four individual carboxy-terminus PTHrP mutants, as well as the alanine combination mutant essentially completely prevented the proliferation driven by the wild-type form of PTHrP. PTHrP carboxy-terminus amino acid residues Ser119, Ser130, Thr132 and Ser138, therefore, are all essential for stimulation of VSM cell proliferation by PTHrP.

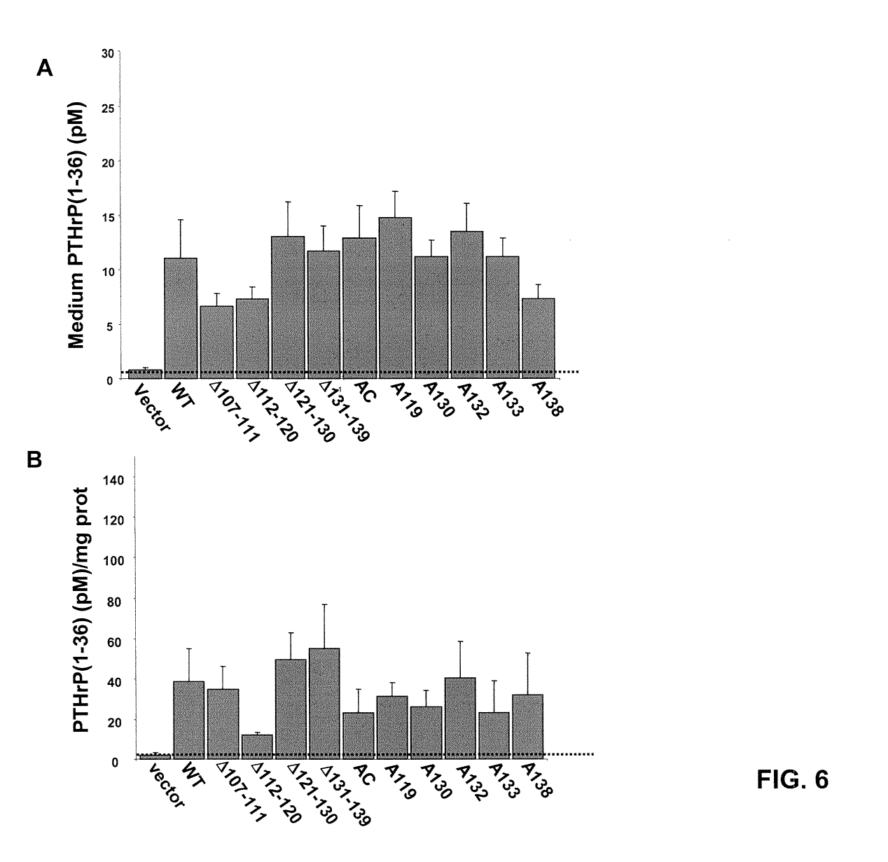

To exclude the possibility that the failure of carboxy-terminus PTHrP mutants to stimulate proliferation in A-10 cells was due to the failure of selected clones to produce PTHrP, three or more clones of each construct employed above were assayed three or four times for their ability to produce PTHrP, examining both cell conditioned medium as well as cell extracts. PTHrP was assayed using a PTHrP(1-36) immunoradiometric assay as described above. As can be seen in FIG. 6, each of the constructs employed led to the production of easily measurable PTHrP (the dotted line indicates the assay detection limit at 0.5 pM), comparable to that observed in the wild-type PTHrP-expressing cells, and each produced far more PTHrP than the vector-transfected cells. The failure of carboxy-terminus PTHrP mutants to stimulate proliferation in A-10 vascular smooth muscle cells was, therefore, not due to ineffective or underexpression of the constructs, since analysis of the conditioned medium and cell extracts indicated that all were expressed at comparable levels (FIG. 6). These results collectively suggest for the first time that serine and threonine residues in the carboxy-terminus of PTHrP have a physiological function and are important targets for post-translational modification, e.g., phosphorylation, O-glycosylation, e.g., N-acetylgalactosamine, and acylation, or other post-translational modification.

### Example 2

### In Vivo Measurement of the Effect of PTHrP Carboxy-Terminal Mutants on Rat Carotid Arterial Neointima Formation

PTHrP mutant polypeptide isolated from a host cell, e.g., A-10 vascular smooth muscle cells, stably transfected with the different PTHrP constructs, e.g., but not limited to, Δ112-120, Δ121-130, Δ131-139, AC-HA, A 119-HA, A 130-HA, A 132-HA, A 133-HA, and A 138-HA PTHrP carboxy terminal mutants; or a polynucleotide encoding Δ112-120, Δ121-130, Δ131-139, AC-HA, A 119-HA, A 130-HA, A 132-HA, A 133-HA, and A 138-HA PTHrP carboxy terminal mutants; or infected by a virus, e.g., but not limited to, adenovirus, containing such PTHrP carboxy-terminus mutant constructs are tested for their effect in a rat model of vessel balloon injury. For example, adult Sprague-Dawley male rats weighing 450-600 g are anesthetized with intraperitoneal injections of ketamine (150 mg/kg body wt) and xylazine (15 mg/kg body wt). Following a neck incision, a 2F Fogarty balloon catheter (Baxter, Irvine, Calif.) is inserted via an arteriotomy into the left common carotid artery. To ensure adequate and reproducible injury, the balloon catheter is inflated with a calibrated inflation device to a pressure of 2 ATM for 5 min. The balloon is passed back and forth three times and removed. A plastic catheter (27gauge½) is introduced through the external carotid arteriotomy, and the common carotid artery is flushed with PBS before introduction of a suitable vehicle alone, vehicle containing PTHrP protein (0.000001 mg protein/kg body weight-100,000 mg protein/kg body weight), or vehicle containing carboxy-terminus mutant PTHrP protein (0.000001 mg protein/kg body weight-100,000 mg protein/kg body weight) or a polynucleotide encoding PTHrP protein (0.000001 mg polynucleotide/kg body weight-100,000 mg polynucleotide/kg body weight), or vehicle containing polynucleotide encoding carboxy terminus mutant PTHrP protein (0.000001 mg polynucleotide/kg body weight-100,000 mg polynucleotide/kg body weight. Alternatively, a viral carrier, e.g., adenovirus, containing an appropriate polynucleotide construct encoding PTHrP (1 pfu/ml to 1×10¹⁴ pfu/ml) or a carboxy-terminus mutant PTHrP (1 pfu/ml to 1×10¹⁴ pfu/ml) is administered.

For example, recombinant adenovirus stocks are used within 2 h of thawing. Fifty microliters of 1 pfu/ml to 1×10¹⁴ pfu/ml adenoviral vector (AdLacZ or Ad-carboxy-terminus PTHrP mutant) or DMEM are instilled into the injured isolated common carotid segment through the plastic catheter. After 15 min, the adenovirus or DMEM is aspirated. The proximal external carotid artery is ligated, and blood flow through the common and Internal carotid is reestablished. Two weeks after balloon injury, the contralateral control artery (which received neither injury nor adenovirus treatment), and the balloon-injured artery with no adenovirus treatment (DMEM) or adenovirus treatment (Ad-LacZ or Ad-carboxy-terminus PTHrP mutant) are harvested and fixed in 4% paraformaldehyde for 48 h at 4° C., embedded in paraffin blocks, sectioned (5 gm), and stained either with hematoxylin and eosin or by Von Giesen method to reveal the internal and external elastic lamina, images are acquired and analyzed for the cross-sectional areas of neointima and media using the NIH Image program, and the area ratio are calculated.

A reduction in the neointima to media ratio in angioplasty-treated vessels receiving carboxy-terminus PTHrP mutant polypeptide compared with the neointima to media ratio observed in angioplasty-treated vessels receiving vehicle alone indicates that the carboxy-terminus mutant PTHrP has an anti-restenosis effect. Similarly, a reduction in the neointima to media ratio in angioplasty-treated vessels receiving a polynucleotide encoding a carboxy-terminus PTHrP mutant polypeptide compared with the neointima to media ratio observed in angioplasty-treated vessels receiving vehicle alone indicates that the polynucleotide encoding the carboxy-terminus mutant PTHrP has an anti-restenosis effect. Moreover, a reduction in the neointima to media ratio observed in angioplasty-treated vessels receiving a viral carrier containing a polynucleotide construct encoding a carboxy-terminus mutant PTHrP compared with the neointima to media ratio observed in angioplasty-treated vessels receiving a viral carrier containing a polynucleotide construct that does not encode a carboxy-terminus mutant PTHrP indicates that the carboxy-terminus mutant PTHrP has an anti-restenosis effect. A Student's T-test is employed to assess differences in the neointima to media ratios observed between treatment groups. “P” values less than or equal to 0.05 are considered significant.

### Example 3

### Cell Cycle Transition into G₁/S in Response to PTHrP in VSM Cells is Associated with Phosphorylation of the Key G₁ Checkpoint Retinonblastoma Protein, PRB

### I. General

In earlier studies, the present inventors had demonstrated that overexpression of wild-type PTHrP in VSM cells increases the rate of cell growth as assessed by cell number and tritiated thymidine incorporation (Massfelder et al., Proc. Natl. Acad. Sd. USA, 94: 13630-635 (1997)). Further, the mitogenic or anti-mitogenic effect of PTHrP is dependent on whether it is secreted from the cell and then activates the PTH/PTHrP receptor by binding to it, or whether the PTHrP is directed via a nuclear localization signal (NLS) to the cell nucleus where it elicits molecular events that stimulate cell proliferation. The NLS is a multibasic amino acid region within the midregion of the PTHrP molecule. The purpose of these studies was to determine the molecular mechanism underlying cell cycle activation by PTHrP overexpression.

### II. Methods

A. Recombinant Adenovirus

Adenovirus encoding β-galactosidase (Invitrogen, Carlsbad, Calif.), human PTHrP (−36 to 139), and human PTHrP with a deleted NLS were prepared as reported previously (Garcia-Ocana et al, J. Biol. Chem., 278: 343-51 (2003)) using Ad.5 constructs generously provided by Dr. Christopher Newgard at the University of Texas Southwestern in Dallas, Tex. (Becker et al. Methods Cell Biol., 43: 161-89 (1994)). Multiplicity of infection (MOI) was determined by spectrophotometrically using OD₂₆₀ and by plaque assay.

B. Cell Cycle Analysis

Cell cycle distribution was analyzed by flow cytometry. Exponentially growing A-10 vascular smooth muscle cells stably transfected with the vector alone, WT-PTHrP or ΔNLS-PTHrP were serum-starved for 72 h. Cells were washed with PBS and incubated with 10% FBS complete DMEM for 24 h. Cells were then harvested, trypsinized, washed with PBS, and incubated in 70% ethanol at 4° C. at least overnight On the day of flow cytometry analysis, fixed cells were washed with PBS, pelleted and resuspended in the staining PBS solution containing 50 μg/ml propidium iodide, 100 U/ml RNAse A and 1 g/L glucose. Stained cells were filtered through a 30 μm nylon mesh and DNA content was analyzed on a Becton-Dickinson flow cytometer.

C. Immunoblot Analysis

Cell extracts were prepared and analyzed by 7.5% SDS-PAGE immunoblotted and transferred to Immobilon-P membranes using standard methods (Stuart et al., Am. J. Physiol. Endocrinol. Metab., 279: E60-7 (2000). For analysis of immunoreactive phosphorylated and dephosphorylated forms of pRb protein, a primary anti-pRb antibody (Pharmingen, San Diego, Calif.) that recognizes both pRb and ppRb was employed. For analyses of immunoreactive a-tubulin, immunoreactive p2′7, and immunoreactive actin protein levels, primary anti-a-tubulin antibody (Oncogene™ Research Products, EMD Bioscience, Inc., San Diego, Calif., USA), primary anti-p27 antibody (Cell Signaling Technology, Beverly, Mass.), and primary anti-actin antibody (Santa Cruz Biotechnology, Inc., Santa Cruz, Calif., USA), were employed, respectively.

D. PTHrP Immunoradiometric Assay

PTHrP secreted from A-10 vascular smooth muscle cells stably transfected with the different PTHrP constructs or infected by the different adenovirus was measured in 24 h conditioned medium obtained at confluence using a two-site immunoradiometric assay (IRMA) specific for PTHrP(1-36) (Massfeider et al., Proc. Natl. Acad. Sci. USA, 94: 13630-635 (1997); De Miguel et al., Endocrinology, 142: 4096-105 (2001)). The detection limit of the assay is 0.5 pM. For measurement of PTHrP in cell extracts, cells were plated in 100 mm culture plates. At confluence, cells were washed with PBS at room temperature and were then resuspended on ice in PBS containing 1% Igepal CA-630 (Sigma, St. Louis, Mo.), 0.5% sodium deoxycholate, 0.1% SDS, 100 μg/ml PMSF, 45 μg/ml aprotinin, and 1 mM sodium orthovanadate. They were sonicated 10 times for 1 sec, incubated on ice for 60 min and then centrifuged at 10,000×g for 10 min at 4° C. The supernatant representing the cell extract was assayed for PTHrP immunoreactivity using the PTHrP (1-36) IRMA described above. Protein was measured according to the method of Bradford, and results are expressed as pmol/mg extract protein.

### III. Overexpression of PTHrP Stimulates PRB Protein Phosphorylation and Overrides Serum-Induced G1/G0 Arrest in A-10 VSM Growth

As shown in FIG. 7 (and reviewed by Hupfeld and Weiss, Am J Physiol Endocrinol Metab 281: E207-E216 (2001), the G1-to-S phase transition in VSM and other cells is accompanied by phosphorylation of the retinoblastoma protein (Rb), releasing its inhibitory effect on the S phase transcription factors, E2F, and resulting in transcription of early genes required for mitosis (Hiebert et al., Genes Dev, 6: 177-18 (1992)). The cyclin-dependent kinases (CDK2, CDK4, and CDK6), in complex with the G1 cyclins (cyclin D1, cyclin E), phosphorylate Rb during G1 and thus set into motion events of cell cycle transit. The cyclin kinase inhibitors (CKIs) have been shown to regulate the activity of cyclin/CDK complexes and thus can have a profound effect on G1-to-S phase progression. The Cip/Kip family of CKIs, which includes p21Waf1/Cip1 and p27Kip1, are capable of inhibiting cyclin/CDK complex activity in G1 phase (Sherr, Cell 73: 1059-1065 (1993)), yet recent work on these molecules has shown that they are, under some conditions, required for assembly of cyclin D- and cyclin E-dependent kinases (Cheng et al., EMBO J. 18: 1571-1583 (1999); Donjerkovic et al., Cell Res 10: 1-16 (2000); Weiss et al., J Biol Chem 275: 28340 (2000).

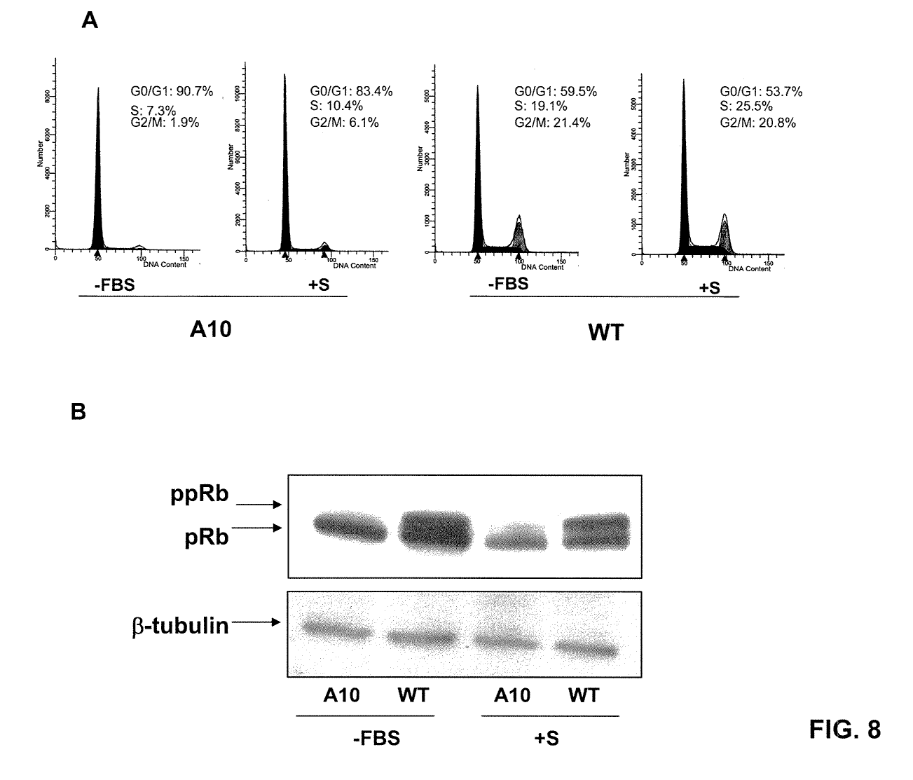

Cell cycle analysis is presented using standard flow cytometric analysis with propidium iodide is shown in FIG. 8A. As can be seen in the figure, 24 hours of serum starvation leads to essentially complete growth arrest in untransfected A-10 cells, and the addition of serum leads to a return of entry into the cell cycle.

That is, A-10 VSM cells grown under conditions of serum starvation proliferate at a slow rate, with the majority of cells being in G0 and small numbers in S and G2M. Following addition of serum, A-10 cells begin to proliferate and the percentage of cells in both S and G2M increases markedly. In contrast, A-10 VSM cells overexpressing wild type PTHrP fail to decelerate under conditions of serum starvation (FIG. 8A), and proliferate at a rate faster than serum replete A-10 VSM cells. Addition of serum does not induce further acceleration of proliferation.

The tumor suppressor protein retinoblastoma protein (pRb) has been shown to be a critical regulator of VSM cell proliferation (Stuart et al., Am J Physiol Endocrinol Metab. 279: E60-7 (2000) and references therein). Phosphorylation and inactivation of pRb in response to mitogenic stimulation results in G₁/S transition and proliferation. The inhibition of pRb phosphorylation results in cells cycle arrest in VSM cells and inhibition of proliferation. Moreover, the phosphorylation of Rb during G₁ progression coincides with the transition through the G₁ restriction point, beyond which cells are committed to DNA synthesis. For these reasons, and because pRb hypophosphorylation has been implicated in the anti-mitogenic effects of extracellular PTHrP(1-36) interacting with its cell surface receptor (Stuart et al., Am J Physiol Endocrinol Metab. 279: E60-7 (2000)), the phosphorylation status of pRb in response to nuclear PTHrP-driven VSM cell proliferation was determined in the present study.

In FIG. 8B, phosphoriation of Rb was examined by Western blot using a pRb antibody (Pharmingen, San Diego, Calif.). In the bottom panel, beta tubulin was seen as a control for loading. As can be seen, in normal A-10 cells, the majority of pRb was in the dephosphorylated form, whether in the serum depleted (−FBS) or serum replete (+S) state. In contrast, A-10 cells overexpressing wild-type PTHrP displayed constitutive phosphorylation of pRb, indicated as ppRb, whether grown under conditions of serum repletion or serum starvation. The observation that overexpression of wild-type PTHrP induces cell cycle progression at the S and G₂/M checkpoints in association with hyperphosphorylation of pRb are in accord with these prior observations, and with our previous results showing stimulation of [³H] thymidine incorporation in A-10 cells overexpressing the WT-PTHrP (Massfelder et al., Proc Natl Acad Sci USA 94: 13630-5 (1997)). These studies show that the nuclear presence of PTHrP leads to pRb phosphorylation.

This also demonstrates that PTHrP acts, at least, in part, via the cyclin D-cdk4, pRb, E2F pathway, and that this action Is independent of serum-derived growth factors. The constitutive phosphorylation of pRb observed in the PTHrP-overexpressing A-10 VSM cells, suggests that PTHrP functions as an upstream activator, for example, of the cyclin D-cdk4 pathway (See also, FIG. 7).

As shown in FIG. 9A, control A-10 VSM cells proliferated at a slow rate, with the majority of cells being in G0 and small numbers in S and G2M. In contrast, A-10 VSM cells stably transfected to overexpress wild-type PTHrP protein proliferated at a greater rate and the percentage of cells in both S and G2M increased markedly compared to control A-10 VSM cells. Consistent with a loss of nuclear targeting, the stable transfection of A-10 VSM cells with PTHrP NLS deletion mutant resulted in a near total arrest of the cells In G1/G0. Indeed, the percentage of cells in S and G2M were lower than the percentage of cells in S and G2M observed in control A-10 VSM cells. As shown in FIG. 9B, the growth arrest observed in the VSM cells that overexpress PTHrP NLS deletion mutant protein correlated with a presence of the pRb protein in a dephosphorylated form.

The p27kip1 protein is increasingly recognized as a pivotal regulatory molecule controlling G1 to S transition (Stuart et al., Am J Physiol Endocrinol Metab, 279: E60-E67 (2000)). In normal cells, p27kip1 levels increase as cells become quiescent and abruptly decline upon cell cycle reentry (Toyoshima et al., Cell, 78: 67-74 (1994)). The induction of p27kip1 also appears to coordinate cell cycle arrest in response to anti-mitogenic stimuli (Durand et al., Curr Biol 8: 431-440 (1998); Matsuo et al., Oncogene 16: 3337-3343 (1998); Polyak et al., Genes Dev 8: 9-22 (1994)). Kato et al. (Cell 79: 487-496 (1994)) first showed that cAMP caused G1 growth arrest in colony-stimulating factor-1-stimulated macrophages by inducing p27kip1 without altering the levels of cyclin D1 or cdk4. Interestingly, studies by Sheaff et al. (Genes Dev 11: 1464-1476 (1997)) provide evidence that the level of p27kip1 is controlled posttranslationally by the cyclin E-cdk2 complex itself. In these studies, the accumulation of cyclin E-cdk2 complexes promoted cell cycle progression by phosphorylation of p27kip, which increased its removal from the cell.

The effect of overexpression of wild-type PTHrP and PTHrP NLS deletion mutant on p27kip1 expression in A-10 VSM cells studied by Western blot analysis (FIG. 10). As shown in FIG. 10, A-10 VSM cells express immunoreactive p27kip1 protein. Overexpression of PTHrP protein In A-10 VSM cells results in a significant reduction of the level of immunoreactive p27kip1 protein compared to the level of immunoreactive p27kip1 protein observed in control A-10 cells. In contrast, engineering of A-10 VSM cells to overexpress PTHrP NLS deletion mutant protein results in a significant increase in the level of immunoreactive p27kip 1 compared to the level of immunoreactive p27kip1 protein observed in either control A-10 VSM cells or A-10 VSM cells that overexpress the wild-type PTHrP protein.

### Example 4

### Adenoviral Gene Delivery of NLS-Deficient PTHrP Inhibits Arterial Restenosis

### I. General

As noted earlier, the present inventors previously demonstrated that whereas intact PTHrP is a potent activator of VSM proliferation, the opposite is true of PTHrP lacking an NLS. PTHrP devoid of its NLS is a potent inhibitor of VSM proliferation (Massfelder et al., Proc Natl Acad Sci USA 94(25): 13630 (1997); de Miguel et al., Endocrinology 142(9): 4096 (2001)). In the following studies, rat and pig models or angioplasty were employed to assess the therapeutic potential of PTHrP NLS deletion mutant in disorders of manifested by smooth muscle proliferation such as vascular restenosis.

### II. Methods

A. Rat Model of Carotid Angioplasty

Balloon injury and adenovirus infection were performed on the left common carotid artery of adult Sprague-Dawley male rats weighing 450-600 g anaesthetized with intraperitoneal injections of ketamine (150 mg/kg body wt) and xylazine (15 mg/kg body wt). Following a neck incision, a 2F Fogarty balloon catheter (Baxter, Irvine, Calif.) was inserted via an arteriotomy into the left common carotid artery. To ensure adequate and reproducible injury, the balloon catheter was inflated with a calibrated inflation device to a pressure of 2 ATM for 5 min. The balloon was passed back and forth three times and removed. A plastic catheter (27½ gauge) was introduced through the external carotid arteriotomy and the common carotid artery was flushed with PBS before introduction of adenovirus. Recombinant adenovirus stocks were used within two hr of thawing. Fifty microliters of 10¹⁰ pfu/ml adenoviral vector (AdLacZ or AdΔNLS) or DMEM was instilled into the injured isolated common carotid segment through the plastic catheter. After 15 min, the adenovirus or DMEM was aspirated. The proximal external carotid artery was ligated, and blood flow through the common and internal carotid was re-established. Two weeks after balloon injury, the contralateral (right) control artery (which received neither injury nor adenovirus treatment), and the balloon-injured (left) artery with no adenovirus treatment (DMEM) or adenovirus treatment (Ad-LacZ or Ad-ΔNLS) were harvested and fixed in 4% paraformaldehyde for 48 h at 4° C., embedded in paraffin blocks, sectioned (5 μm), and stained either with hematoxylin and eosin or by Von Giesen method to reveal the internal and external elastic lamina. Images were acquired and analyzed for the cross-sectional areas of neointima and media using the NIH Image program, and the area ratio was calculated. A Student's T-test was employed to assess the statistical significance of the intima to media ratios observed the treatment groups. A “P” value less than or equal to 0.05 were considered significant.

B. Pig Arterial Injury Model

Adenovirus-mediated gene transfer was performed in the iliac arteries of domestic Hampshire pigs (15 kg), with adenovirus containing an NLS PTHrP mutant gene (Ad-ΔNLS) or with a reporter gene (Ad-LacZ). After sedation with Ketamine (20 mg/kg body wt) and xylazine (2 mg/kg), the pigs were intubated and anesthetized with Isoflurane/NO. Under sterile surgical techniques, a #3 French-balloon catheter was inserted into the iliac artery through the internal iliac artery and inflated to 2 atm for 5 min. The arterial segment was rinsed with 5 mL saline solution. Recombinant adenovirus stocks were used within 2 h of thawing. One ml of 10¹⁰ pfu/ml adenoviral vector (Ad-LacZ or Ad-ΔNLS) or DMEM was instilled into the injured isolated iliac artery segment through the plastic catheter. After 30 min, the adenovirus or DMEM was aspirated. After adenovirus treatment, the catheter was removed, and arterial flow was restored. Animals were killed three weeks after adenovirus treatment and the angioplastied segments of the iliac arteries were harvested along with more distal segments used as negative, normal control segments.

The harvested vessel tissue was fixed in 4% paraformaldehyde for 48 h at 4° C., embedded in paraffin blocks, sectioned (5 μm), and stained either with hematoxylin and eosin or by Von Giesen method to reveal the internal and external elastic lamina. Images were acquired and analyzed for the cross-sectional areas of neointima and media using the NIH Image program, and the area ratio was calculated. A Student's T-test was employed to assess the statistical significance of the intima to media ratios observed the treatment groups. A “P” value less than or equal to 0.05 were considered significant.

C. Recombinant Adenoviral Vectors.

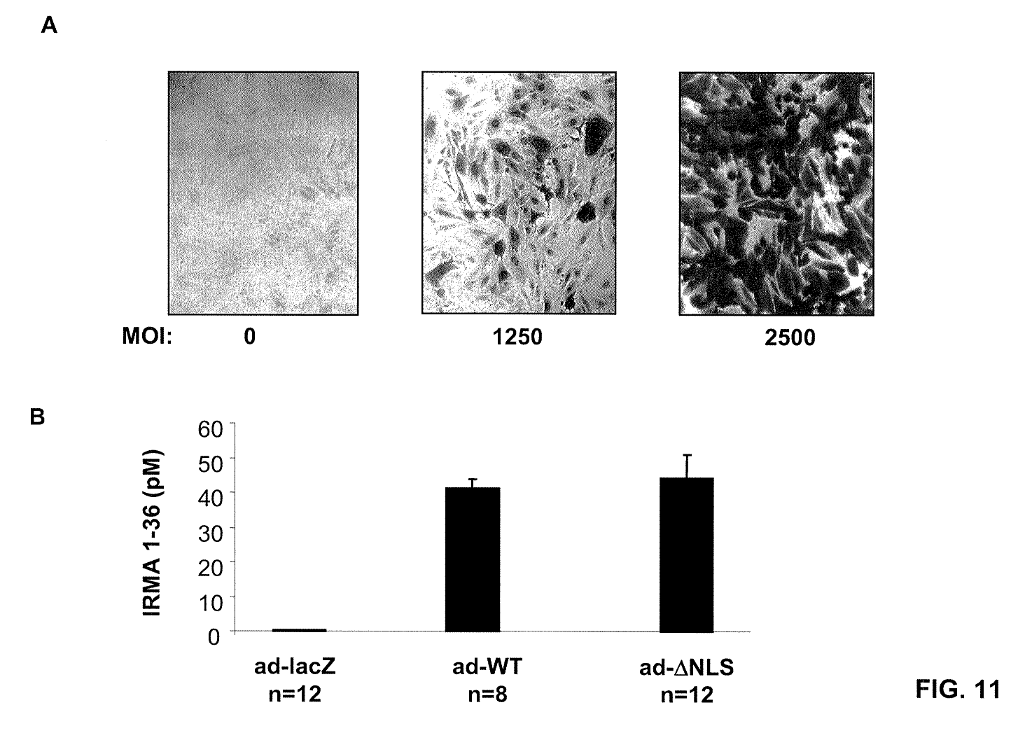

Replication-defective Ad5 adenovirus deleted for Ela and Elb obtained from Dr. Chris Newgard at Duke University was engineered to express beta-galactosidase (AdLacZ), wild-type PTHrP (Ad-WT) or PTHrP deleted for the NLS (AdΔNLS) constructs were used in these studies. Three replication-deficient, recombinant adenoviral vectors were constructed, propagated, and purified as described by Becker and coworkers (Becker et al., Meth. Cell Biol. 43: 161-89 (1994)). Confirmation of the sequences was accomplished by DNA sequencing. These vectors were prepared from adenovirus-5 serotype and contain deletions in E1 and E3 regions, rendering them replication incompetent. The three adenoviral vectors (Ad) include a vector encoding PTHrP lacking the NLS sequence (AdΔNLS), driven by a CMV promoter and enhancer. An adenoviral vector lacking a cDNA insert, AdLacZ, was used for control experiments. A third adenoviral vector, Ad-WT, encodes for wild-type PTHrP. Viral stocks were sterilized with a 0.45-um filter and evaluated for the presence of replication-competent virus by infection of A-10 VSM cells at an MOI of 2500 (See FIG. 11). None of the stocks used in these experiments yielded replication-competent virus. Viral stocks were diluted to titers of 10¹⁰-10¹⁴ plaque-forming units (pfu)/ml, stored at 20° C., and thawed on ice before use.

### III. Adenoviral Gene Delivery of NLS-Deficient PTHrP Inhibits Arterial Restenosis in a Rat Model of Arterial Injury

In the rat carotid, PTHrP gene expression in VSM cells markedly Increases during neointimal formation in response to balloon angioplasty (Stuart et al., Am J Physio/Endocrinol Metab. 279:E60-7 (2000)). In human coronary arteries, VSM cells at sites of coronary atherosclerosis overexpress PTHrP (Nakayama et al., Biochem Biophys Res Commun. 200:1028-35 (1994)). Ishikawa et al., (Atherosclerosis. 152: 97-105 (2000)) have recently demonstrated that local administration of PTHrP(1-34) inhibits intimal thickening induced by a non-obstructive polyethylene cuff in an rat iliac artery model of arterial injury. These observations imply that PTHrP produced locally within the arterial wall may play a role in the arterial response to injury, but do not define what such a role might be. Our prior observation that PTHrP devoid of the NLS is a potent inhibitor of VSM proliferation prompted the question as to whether ΔNLS-PTHrP delivered adenovirally to the arterial wall at the time of carotid angioplasty might have therapeutic efficacy in preventing the neointimal hyperplasia.

Initial studies were performed to confirm that adenovirus expressing beta-galactosidase (AdLacZ), wild-type PTHrP (AdWT) or PTHrP deleted for the NLS (AdΔNLS) efficiently transduce A-10 VSM cells in culture and are summarized in FIG. 11. As shown in FIG. 11A, the AdLacZ virus was introduced at a multiplicity of infection (MOI) of 0, 1250 or 2500 to cultured rat A-10 VSM cells for 15 minutes, and beta-galactosidase was visualized 48 hours later using standard methods. Robust expression of beta-galactosidase activity was observed with infection of the A-10 VSM cells at 2500 MOI, therefore, all three viruses were introduced to A-10 VSM cells for 15 minutes at 2500 MOI, and PTHrP production was examined 48 hours later and quantified by PTHrP immunoradiometric assay as detailed previously (FIG. 11B; see also Example 1). Deletion of the NLS prevents nuclear entry of PTHrP (Massfelder et al., Proc Natl Acad Sci USA 94(25): 13630 (1997); de Miguel et al., Endocrinology 142(9): 4096 (2001)) but does not prevent production or secretion of the PTHrP(1-36) region of the peptide. Thus, this assay can serve as a measure of production of PTHrP by the NLSdeletlon construct. As shown in FIG. 11B, infection of A-10 VSM cells with both the wild-type and NLS-deleted form of PTHrP leads to production of measurable PTHrP production in these cells. That is, the AdLacZ or AdΔNLS adenovirus vectors were effective and efficient at transducing A-10 VSM cells in culture.

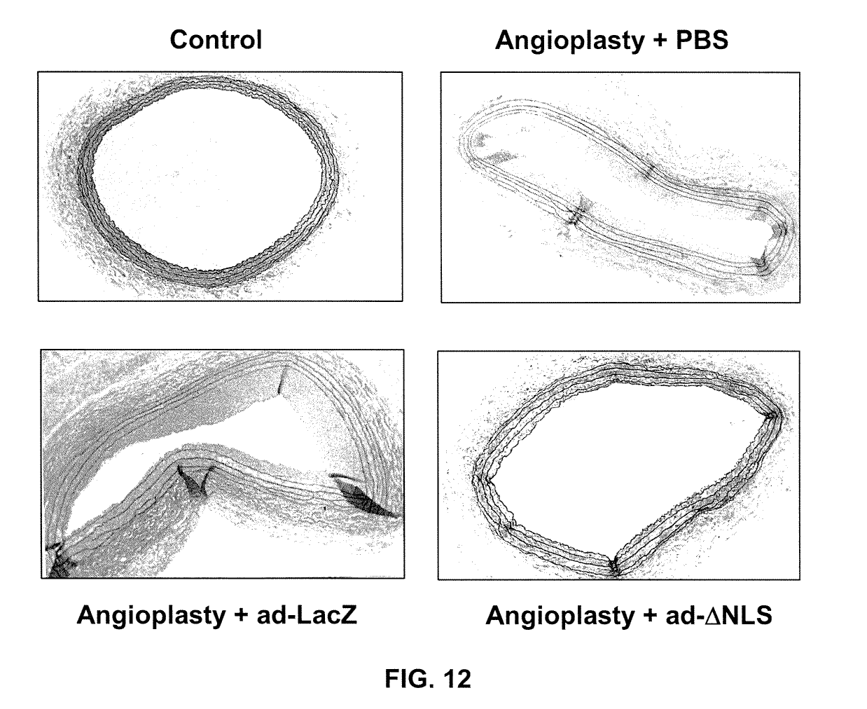

In order to assess the effect of overexpression of Ad-delta-NLS-PTHrP on arterial restenosis in vivo, a standard rat carotid angioplasty restenosis model was employed as detailed above (see Methods Section). As shown in FIG. 12B, balloon angioplasty induced marked arterial restenosis and neointima formation, not present In the contralateral control carotid (FIG. 12A). Similarly, angioplasty followed by the administration or AdLacZ resulted in comparable degrees of restenosis and neointima formation (FIG. 12C). In dramatic contrast, angioplasty followed by the administration of Ad-delta-NLS-PTHrP adenovirus essentially completely prevented arterial restenosis in this model (FIG. 12D).

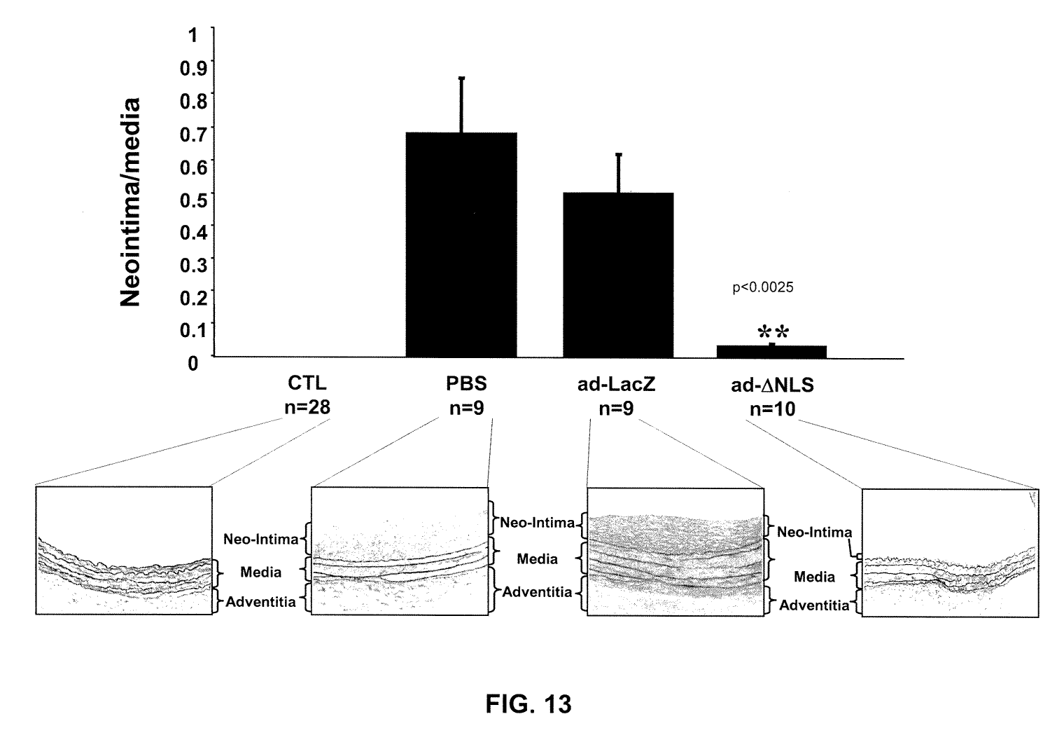

Replication of these studies allowed for the statistical assessment summarized below in Table 1 and represented graphically in FIG. 13.

TABLE 1

| | Neointima | Media Area | Neointima/Media |
|---|---|---|---|
| | Area (mm2) | (mm2) | Ratio |
| Control Carotid, | 0.00 | 0.145 +/− 0.011 | 0.00 |
| No Angioplasty (n = 28) | | | |
| Angioplasty with | 0.099 +/− 0.018 | 0.142 +/− 0.010 | 0.68 +/− 0.17 |
| No adenovirus (n = 9) | | | |
| Angioplasty with | 0.098 +/− 0.020 | 0.209 +/− 0.042 | 0.50 +/− 0.12 |
| Ad-lacZ (n = 9) | | | |
| Angioplasty with | 0.006 +/− 0.002* | 0.181 +/− 0.017 | 0.03 +/− 0.01** |
| Ad-delta-NLS-PTHrP (n = 10) | | | |

* = p < 0.0025;
** = p < 0.0001

Angioplasty alone, or angioplasty followed by treatment with AdLacZ resulted in marked neointima formation. In contrast, angioplasty followed by treatment with Ad-delta-NLS-PTHrP essentially completely (95%) prevented arterial restenosis. Taken together, these studies demonstrate that PTHrP, specifically the NLS-deleted form, has a therapeutic benefit in disorders associated with arterial smooth muscle cell proliferation, migration and matrix secretion. This approach can be employed as well in treating human coronary and peripheral arterial disease.

As hypothesized, the delivery of ΔNLS-PTHrP using an adenoviral construct at the time of angioplasty profoundly suppressed the development of neointimal hyperplasia following arterial injury. This inhibitory response to neointimal development was quantitatively large, statistically significant and highly reproducible. The effect could be attributed only to the ΔNLS-PTHrP, since parallel administration of an Ad-lacZ virus had no independent effect. Importantly, the method of ΔNLS-PTHrP delivery for 15 minutes at the time of angioplasty is one that is possible to use in humans undergoing angioplasty.

While not wishing to be bound by theory, there are two general hypotheses for the mechanism through which Ad-ΔNLS inhibit neointima formation. First, deleting of the NLS in PTHrP prevents nuclear targeting of PTHrP, and thus prevents its ability to drive the cell cycle. However, as documented above, overexpression of ΔNLS-PTHrP also leads to enhanced secretion of PTHrP(1-36). As noted above, PTHrP(1-36), acting on the G-coupled PTH1-receptor on VSM cells to stimulate adenylyl cyclase, Is a potent inhibitor of VSM proliferation (Massfelder and Helwig, Endocrinology. 140: 1507-10 (1999); Clemens et al., Br J Pharmacol. 134:1113-36 (2001); Massfelder et al., Proc Natl Acad Sci USA 94: 13630-5 (1997); Stuart et al., Am J Physiol Endocrinol Metab. 279: E60-7 (2000)). Thus, in this scenario, overexpression of ΔNLS-PTHrP would lead to two outcomes: ablation of the nuclear-PTHrP stimulus to VSM proliferation, and enhancement of PTHrP(1-36) secretion resulting in cell surface PTH1-receptor-mediated inhibition of VSM proliferation. Theoretically, a second scenario could also be operative where ΔNLS-PTHrP overexpression acts in a dominant negative fashion. In such a scenario, ΔNLS-PTHrP could serve to prevent endogenous PTHrP from entering the nucleus and prevent endogenous PTHrP from stimulating cell cycle progression.

IV. adenoviral Gene Delivery of NLS-Deficient PTHrP Inhibits Arterial Restenosis in a Pig Model of Arterial Injury

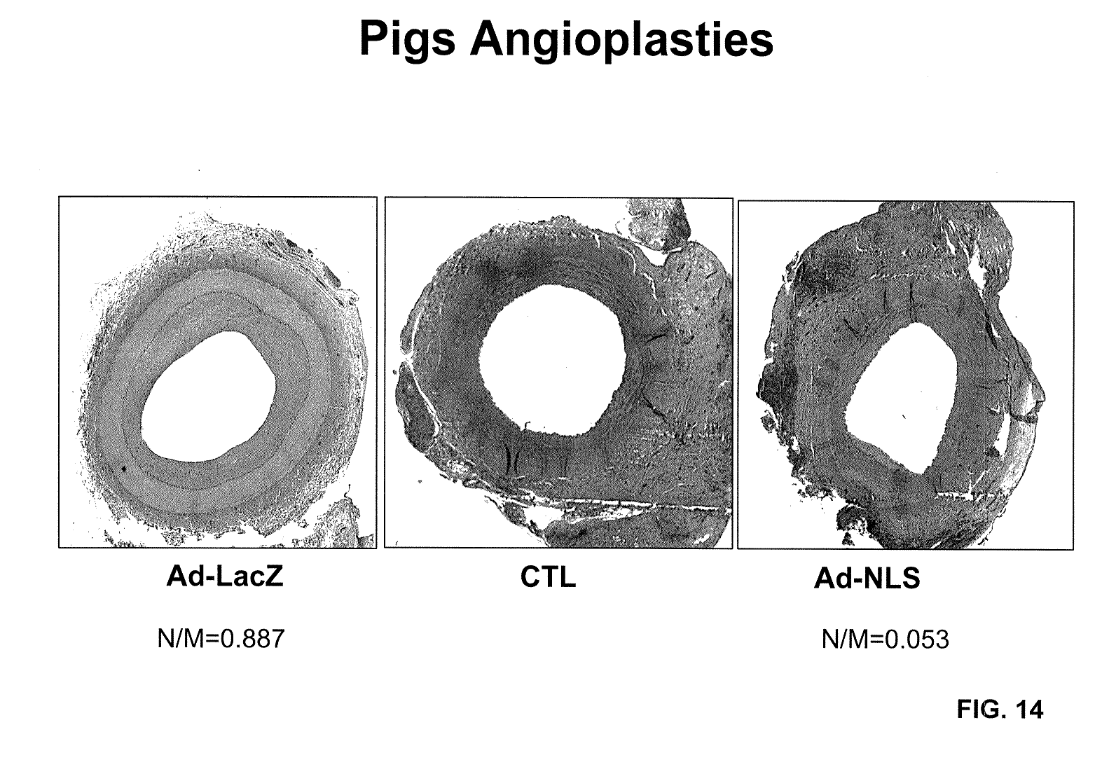

In order to assess the effect of overexpression of Ad-delta-NLS-PTHrP on arterial restenosis in vivo, a pig iliac artery restenosis model was employed as detailed above (see Methods Section). As shown in FIG. 14, middle panel, balloon angioplasty induced marked arterial restenosis and neointima formation. Similarly, angioplasty followed by the administration of AdLacZ resulted in comparable degrees of restenosis and neointima formation (FIG. 14, left panel). In dramatic contrast, angioplasty followed by the administration of AdΔNLS adenovlrus essentially completely prevented arterial restenosis in this model (FIG. 14, right panel). Indeed, administration of AdΔNLS adenovirus resulted in greater than 90% reduction in the neointima to media ratio (N/M=0.053) compared with the neointima to media ratio observed in vessels treated with the AdLacZ adenovirus (N/M=0.887). This study demonstrates that PTHrP, specifically the NLS-deleted form, has a therapeutic benefit in disorders associated with arterial smooth muscle cell proliferation, migration and matrix secretion. Further, these finds confirm the observations made in the rat model of arterial injury.

### Example 5

### In Vivo Measurement of the Effect of PTHrP Mutants on Rabbit Atherosclerosis

PTHrP mutant polypeptide isolated from host cells, e.g., A-10 vascular smooth muscle cells, stably transfected with the different PTHrP constructs, e.g., but not limited to, Δ112-120, Δ121-130, Δ131-139, AC-HA, A 119-HA, A 130-HA, A 132-HA, A 133-HA, A 138-HA PTHrP carboxy terminal mutants, and NLS PTHrP deletion mutant; or polynucleotide encoding Δ112-120, Δ121-130, Δ131-139, AC-HA, A 119-HA, A 130-HA, A 132-HA, A 133-HA, A 138-HA PtHrP carboxy terminal mutants, or NLS PTHrP deletion mutant; or infected by a virus, e.g., but not limited to, adenovirus, containing such PTHrP mutant constructs are tested for their effect in a rabbit model of atherosclerosis as described by Simari and coworkers (Simari et al., Clin. Invest., 98: 225-35 (1996). Briefly, NZW rabbits are sedated with ketamine (35 mg/kg i.m.) and xylazine (5 mg/kg i.m.) and intubated. Anesthesia is maintained with isoflurane. Before surgery, blood chemistries and serum cholesterol and triglyceride levels are measured (Roche Biomedlcal Laboratories, Nutley, N.J.). Surgical exposure and arteriotomy of the right femoral artery is performed, and a 3-French Fogarty balloon catheter (Baxter Healthcare Corp., Mundelein, Ill.) is passed into the common iliac artery. The balloon is inflated in the right iliac artery and withdrawn three times. The right femoral artery is ligated distally, and the incision is closed. After surgery, the rabbits are fed a high fat diet consisting of 0.5% cholesterol and 2.3% peanut oil until they are killed. All animals received aspirin, 10 mg/kg, three times a week. Two rabbits are killed 3 wk after denuding injury and cholesterol feeding, and the iliac arteries are analyzed to determine the extent of aineroscierotic lesions.

Three weeks after the first vascular injury, an angioplasty balloon injury is performed in the right iliac artery. Serum cholesterol and triglyceride levels are measured. A midline abdominal incision is made, and the distal aorta and iliac common arteries are isolated. Side branches in the iliac arteries are isolated and ligated. A 2-2.75-mm balloon angioplasty catheter (SciMed, BSC, Maple Grove, Minn.) is advanced via a distal aortotomy into the right iliac artery. The angioplasty balloon is inflated to six atmospheres of pressure for 1 min and deflated. Balloon inflation and deflation is repeated two times.

Treatment of the vessel with test agent is performed by withdrawing the balloon catheter to a position just proximal to the injury site. The arterial segment is isolated with temporary ligatures and rinsed with 5 ml of phosphate-buffered saline before introduction of a suitable vehicle alone, vehicle containing PTHrP protein (0.000001 mg protein/kg body weight-100,000 mg protein/kg body weight), or vehicle containing mutant PTHrP protein (0.000001 mg protein/kg body weight-100,000 mg protein/kg body weight); or the polynucleotide encoding containing PTHrP protein (0.000001 mg polynucleotide/kg body weight-100,000 mg polynucleotide/kg body weight), or vehicle containing polynucleotide encoding mutant PTHrP protein (0.000001 mg polynucleotide/kg body weight-100,000 mg polynucleotide/kg body weight. Alternatively, a viral carrier, e.g., adenovirus, containing an appropriate polynucleotide construct encoding PTHrP (1 pfu/ml to 1×10¹⁴ pfu/ml) or a mutant PTHrP (1 pfu/ml to 1×10¹⁴ pfu/ml) is administered. For example, recombinant adenovirus stocks are used within 2 h of thawing. Fifty microliters of 1 pfu/ml to 1×10¹⁴pfu/ml adenoviral vector (AdLacZ or Ad-PTHrP mutant) or DMEM are instilled into the injured isolated common carotid segment through the plastic catheter. After 15 min, the adenovirus or DMEM is aspirated. The proximal external carotid artery is ligated, and blood flow through the common and internal carotid is reestablished.

Three weeks after treatment, the contralateral control artery (which received neither injury nor adenovirus treatment), and the balloon-injured artery with no adenovirus treatment (DMEM) or adenovirus treatment (Ad-LacZ or Ad-PTHrP mutant) are harvested and fixed in 4% paraformaldehyde for 48 h at 4° C., embedded in paraffin blocks, sectioned (5 μm), and stained either with hematoxylin and eosin or by Von Giesen method to reveal the internal and external elastic lamina. Images are acquired and analyzed for the cross-sectional areas of neointima and media using the NIH Image program, and the area ratio are calculated.

A reduction in the neointima to media ratio in angioplasty-treated vessels receiving PTHrP mutant polypeptide compared with the neointima to media ratio observed In angioplasty-treated vessels receiving vehicle alone indicates that the PTHrP mutant polypeptide has an anti-restenosis effect. Similarly, reduction in the neointima to media ratio in angioplasty-treated vessels receiving polynucleotide encoding PTHrP mutant polypeptide compared with the neointima to media ratio observed in angioplasty-treated vessels receiving vehicle alone indicates that the PTHrP mutant polypeptide has an anti-restenosis effect. Moreover, a reduction in the neointima to media ratio observed in angioplasty-treated vessels receiving a viral carrier containing a polynucleotide construct encoding a mutant PTHrP compared with the neointima to media ratio observed in angioplasty-treated vessels receiving a viral carrier containing a polynucleotide construct that does not encode a mutant PTHrP indicates that the mutant PTHrP has an anti-restenosis effect. A Students T-test is employed to assess differences in the neointima to media ratios observed between treatment groups. “P” values less than or equal to 0.05 are considered significant.

### Example 6

### In Vivo Measurement of the Effect of PTHrP Mutants Delivered by a Stent on Rabbit Atherosclerosis

PTHrP mutant polypeptide isolated from host cells, e.g., A-10 vascular smooth muscle cells, stably transfected with the different PTHrP constructs, e.g., but not limited to, Δ112-120, Δ121-130, Δ131-139, AC-HA, A 119-HA, A 130-HA, A 132-HA, A 133-HA, A 138-HA PtHrP carboxy terminal mutants, and NLS PTHrP deletion mutant; or polynucleotide encoding Δ112-120, Δ121-130, Δ131-139, AC-HA, A 119-HA, A 130-HA, A 132-HA, A 133-HA, A 138-HA PtHrP carboxy terminal mutants, or NLS PTHrP deletion mutant; or infected by a virus, e.g., but not limited to, adenovirus, containing such PTHrP mutant constructs are tested for their effect being delivered by a stent in a rat model of vessel balloon injury (Rogers et al., Circulation 91:2995-3001 (1995)).

New Zealand White rabbits (Millbrook Farm Breeding Labs) weighing 3 to 4 kg, housed individually in steel mesh cages and fed rabbit chow and water ad libitum, are anesthetized with 35 mg/kg IM ketamine (Aveco Co) and 4 mg/kg IV sodium Nembutal (Abbott Laboratories). Each femoral artery is exposed and ligated, and iliac arterial endothelium is removed by a 3F balloon embolectomy catheter (Baxter Healthcare Corp. Edwards Division) passed via arteriotomy retrograde into the abdominal aorta and withdrawn inflated three times. A 7 mm long, stainless steel stent with a configuration of a series of corrugated rings connected by short longitudinal bridges (MULTI-LINK, Advanced Cardiovascular Systems) is mounted coaxially on a 3-mm angioplasty balloon (Advanced Cardiovascular Systems) and passed retrograde via arteriotomy into each iliac artery, and expand with A-10-second inflation at 2- to 10-atm pressure. Four stents are coated with 3-μm-thick coating of 25% (w/v) pluronic F-127 gel solution (BASF Wyandotte Co., Wyandotte, Mich., USA) and another four stents are coated with the same gel solution with a vehicle containing PTHrP protein (0.000001 mg protein/ml-100,000 mg protein/kg body weight) dissolved in it and another four stents are coated with the same gel solution with a vehicle containing mutant PTHrP protein (0.000001 mg protein/ml-100,000 mg protein/ml) dissolved in it. Alternatively, four stents are coated with 3-μm-thick coating of 25% (w/v) pluronic F-127 gel solution (BASF Wyandotte Co., Wyandotte, Mich., USA) and another four stents are coated with the same gel solution with a vehicle containing a polynucleotide encoding PTHrP protein (0.000001 mg polynucleotide/ml -100,000 mg polynucleotide/ml) dissolved in it and another four stents are coated with the same gel solution with a vehicle containing a polynucleotide encoding mutant PTHrP protein (0.000001 mg polynucleotide/ml-100,000 mg polynucleotide/ml) dissolved in it. A viral carrier, e.g., adenovirus, containing an appropriate polynucleotide construct encoding PTHrP (1 pfu/ml to 1×10¹⁴ pfu/ml) or a carboxy-terminus mutant PTHrP (1 pfu/ml to 1×10¹⁴ pfu/ml) is mixed in the gel. Four iliac arteries subject only to balloon withdrawal injury without stent placement are also harvested and processed for histological analysis.

Begin rabbits on aspirin (Sigma Chemical Co) 0.07 mg/mL in drinking water 1 day before surgery to achieve an approximate dose of 5 mg/kg per day for the duration of the experiment and received a single bolus of standard anticoagulant heparin (100 U/kg, Elkin-Sinn Inc) intravenously at the time of surgery.

Two weeks after balloon injury, iliac arteries are harvested. Under deep anesthesia with intravenous sodium Nembutal, inferior vena caval exsanguination is followed by perfusion with lactated Ringer's solution via left ventricular puncture. Both iliac arteries are excised and fixed in 4% paraformaldehyde for 48 h at 4° C., embedded in paraffin blocks, sectioned (5 gm), and stained either with hematoxylin and eosin or by Von Giesen method to reveal the internal and external elastic lamina. Images are acquired and analyzed for the cross-sectional areas of neointima and media using the NIH Image program, and the area ratio are calculated.

A reduction in the neointima to media ratio in angioplasty-treated vessels receiving stent coated with gel including PTHrP mutant polypeptide compared with the neointima to media ratio observed in angioplasty-treated vessels receiving stent plus gel with vehicle or stent plus gel alone, or stent alone, indicates that the PTHrP mutant polypeptide has an anti-restenosis effect Similarly, reduction in the neointima to media ratio in angioplasty-treated vessels receiving stent coated with gel including polynucleotide encoding PTHrP mutant polypeptide compared with the neointima to media ratio observed in angioplasty-treated vessels receiving stent plus gel with vehicle, or stent plus gel alone, or stent alone, indicates that the polynucleotide encoding the PTHrP mutant polypeptide has an anti-restenosis effect. Moreover, a reduction in the neointima to media ratio observed in angioplasty-treated vessels receiving a stent coated with a gel mixed with a viral carrier containing a polynucleotide construct encoding a PTHrP mutant polypeptide compared with the neointima to media ratio observed in angioplasty-treated vessels receiving stent coated with a gel mixed with a viral carrier containing a polynucleotide construct that does not encode a mutant PTHrP polypeptide indicates that the viral carrier containing a polynucleotide encoding a mutant PTHrP has an anti-restenosis effect. A Student's T-test is employed to assess differences in the neointima to media ratios observed between treatment groups. “P” values less than or equal to 0.05 are considered significant.

### Example 7

### Preparation of SMCM-Coated Devices

Reagents and equipment which are utilized within the following experiments include (medical grade stents obtained commercially from a variety of manufacturers; e.g. the “Strecker” stent) and holding apparatus, 20 ml glass scintillation vial with cap (plastic insert type), TLC atomizer, Nitrogen gas tank, glass test tubes (various sizes from 1 ml and up), glass beakers (various sizes). Pasteur pipette, tweezers, Polycaprolactone (“PCL”-mol wt 10,000 to 20,000; Polysciences), SMCM compound, e.g., PTHrP mutant polypeptide isolated from host cells, e.g., A-10 vascular smooth muscle cells, stably transfected with the different PTHrP constructs, e.g., but not limited to, Δ112-120, Δ121-130, Δ131-139, AC-HA, A 119-HA, A 130-HA, A 132-HA, A 133-HA, A 138-HA PtHrP carboxy terminal mutants, and NLS PTHrP deletion mutant; or polynucleotide encoding Δ112-120, Δ121-130, Δ131-139, AC-HA, A 119-HA, A 130-HA, A 132-HA, A 133-HA, A 138-HA PtHrP carboxy terminal mutants, or NLS PTHrP deletion mutant; or infected by a virus, e.g., but not limited to, adenovirus, containing such PTHrP mutant constructs, Ethylene vinyl acetate (“EVA”-washed-see previous). Poly (DL) lactic acid (“PLA”-mol wt 15,000 to 25,000; Polysciences), dichloromethane (“DCM”-HPLC grade, Fisher Scientific). It is to be understood that these procedures can be used to coat the surface of many different types of devices, e.g., but not limited to, as stents and catheters.

A. Procedure for Sprayed Stents

The following describes a typical method using a 3 mm crimped diameter interleaving metal wire stent of approximately 3 cm length. For larger diameter stents, larger volumes of polymer/drug solution are used. Briefly, a sufficient quantity of polymer is weighed directly into a 20 ml glass scintillation vial, and sufficient DCM added in order to achieve a 2% w/v solution. The vial is then capped and mixed by hand in order to dissolve the polymer. The stent is then assembled in a vertical orientation, tying the stent to a retort stand with nylon. Position this stent holding apparatus 6 to 12 inches above the fume hood floor on a suitable support (e.g., inverted 2000 ml glass beaker) to enable horizontal spraying. Using an automatic pipette, a suitable volume (minimum 5 ml) of the 2% polymer solution is transferred to a separate 20 ml glass scintillation vial. An appropriate amount of SMCM compound, PTHrP mutant polypeptide isolated from host cells, e.g., A-10 vascular smooth muscle cells, stably transfected with the different PTHrP constructs, e.g., but not limited to, Δ112-120, Δ121-130, Δ131-139, AC-HA, A 119-HA, A 130-HA, A 132-HA, A 133-HA, A i 36-HA PtHrP carboxy terminal mutants, and NLS PTHrP deletion mutant; or polynucleotide encoding Δ112-120, Δ121-130, Δ131-139, ΔC-HA, A 119-HA, A 130-HA, A 132-HA, A 133-HA, A 138-HA PtHrP carboxy terminal mutants, or NLS PTHrP deletion mutant; or infected by a virus, e.g., but not limited to, adenovirus, containing such PTHrP mutant constructs, is then added to the solution and dissolved by hand shaking

To prepare for spraying, remove the cap of this vial and dip the barrel (only) of an TLC atomizer into the polymer solution. Note that the reservoir of the atomizer need not be used in this procedure: the 20 ml glass vial acts as a reservoir. Connect the nitrogen tank to the gas inlet of the atomizer. Gradually increase the pressure until atomization and spraying begins. Note the pressure and use this pressure throughout the procedure. To spray the stent use 5 second oscillating sprays with a 15 second dry time between sprays. After 5 sprays, rotate the stent 90° and spray that portion of the stent. Repeat until all sides of the stent have been sprayed. During the fry time, finger crimp the gas line to avoid wastage of the spray. Spraying is continued until a suitable amount of polymer is deposited on the stents. The amount may be based on the specific stent application in vivo. To determine the amount, weigh the stent after spraying has been completed and the stent has dried. Subtract the original weight of the stent from the finished weight and this produces the amount of polymer (plus paclitaxel) applied to the stent. Store the coated stent in a sealed container.

B. Procedure for Dipped Stents

The following describes a typical method using a 3 mm crimped diameter interleaving metal wire stent of approximately 3 cm length. For larger diameter stents, larger volumes of polymer/drug solution are used in larger sized test tubes.

Weigh 2 g of EVA into a 20 ml glass scintillation vial and add 20 ml of DCM. Cap the vial and leave for 2 hours to dissolve (hand shake the vial frequently to assist the dissolving process). Weigh a known weight of paclitaxel directly into a 1 ml glass test tube and add 0.5 ml of the polymer solution. Using a glass Pasteur pipette, dissolve the PTHrP mutant polypeptide isolated from host cells, e.g., A-10 vascular smooth muscle cells, stably transfected with the different PTHrP constructs, e.g., but not limited to, Δ112-120, Δ121-130, Δ131-139, AC-HA, A 119-HA, A 130-HA, A 132-HA, A 133-HA, A 138-HA PtHrP carboxy terminal mutants, and NLS PTHrP deletion mutant; or polynucleotide encoding Δ112-120, Δ121-130, A 131-139, AC-HA, A 119-HA, A 130-HA, A 132-HA, A 133-HA, A 138-HA PtHrP carboxy terminal mutants, or NLS PTHrP deletion mutant; or infected by a virus, e.g., but not limited to, adenovirus, containing such PTHrP mutant constructs by gently pumping the polymer solution. Once the materials are suitably mixed or dissolved, hold the test tube in a near horizontal position (the sticky polymer solution will not flow out). Using tweezers, insert the stent into the tube all the way to the bottom. Allow the polymer-containing solution to flow almost to the mouth of the test tube by angling the mouth below horizontal and then restoring the test tube to an angle slightly above the horizontal. While slowly rotating the stent in the tube, slowly remove the stent (approximately 30 seconds).

Hold the stent in a vertical position to dry. Some of the sealed perforations may pop so that a hole exists in the continuous sheet of polymer. This may be remedied by repeating the previous dipping procedure, however repetition of the procedure can also lead to further popping and a general uneven build up of polymer. Generally, it is better to dip the stent just once and to cut out a section of stent that has no popped perforations. Store the dipped stent in a sealed container until use.

### EQUIVALENTS

From the foregoing detailed description of the specific embodiments of the invention, it should be apparent that unique bioactive peptides have been described. Although particular embodiments have been disclosed herein in detail, this has been done by way of example for purposes of illustration only, and is not intended to be limiting with respect to the scope of the appended claims which follow. In particular, it is contemplated by the inventors that various substitutions, alterations, and modifications may be made to the invention without departing from the spirit and scope of the invention as defined by the claims. For instance, the choice of SMCM analog, or the route of administration is believed to be matter of routine for a person of ordinary skill in the art with knowledge of the embodiments described herein.

We claim:

1. A compound comprising a parathyroid hormone-related protein mutant polypeptide wherein the compound has the following characteristics:
(a) the compound lacks a functional nuclear localization signal;
(b) overexpressing the compound in a vascular smooth muscle cell decreases the level of phosphorylated immunoreactive retinoblastoma polypeptide compared to the to the level of phosphorylated immunoreactive retinoblastoma polypeptide observed in the absence of the compound; and
(c) overexpressing the compound in a vascular smooth muscle cell increases the level of immunoreactive p27kip1 polypeptide compared to the level of immunoreactive p27kip1 polypeptide observed in the absence of the compound.

2. An isolated nucleic acid encoding compound of claim 1.

3. A vector comprising the nucleic acid of claim 2.

4. The vector of claim 3, further comprising a promoter operably linked to the nucleic acid molecule.

5. A cell comprising the vector of claim 4.

6. A virus comprising the vector of claim 4.

7. The virus of claim 6, wherein the virus is adenovirus.

8. A pharmaceutical composition comprising a compound of claim 1, and a pharmaceutically acceptable carrier.

9. An antibody or fragment thereof that binds immunospecifically to a compound of claim 1.

10. The antibody of claim 9, wherein the antibody is a monoclonal antibody.

11. The antibody of claim 10, wherein the antibody is a humanized antibody.

12. A pharmaceutical composition comprising an antibody of claim 11, and a pharmaceutically acceptable carrier.

13. A pharmaceutical composition comprising the nucleic acid molecule of claim 4 and a pharmaceutically-acceptable carrier.

14. A pharmaceutical composition comprising the virus of claim 6.

15. A method for preparing a compound, the method comprising:
(a) culturing a cell containing a nucleic acid according to claim 5 under conditions that provide for expression of the compound; and
(b) recovering the expressed compound.

16. A method for determining the presence or amount of the compound of claim 1 in a sample, the method comprising:
(a) providing the sample;
(b) contacting the sample with an antibody that binds immunospecifically to the compound; and
(c) determining the presence or amount of antibody bound to the compound, thereby determining the presence or amount of compound in the sample.

17. A method for determining the presence or amount of the nucleic acid molecule of claim 2 in a sample, the method comprising:
(a) providing the sample;
(b) contacting the sample with a probe that binds to the nucleic acid molecule; and
(c) determining the presence or amount of the probe bound to the nucleic acid molecule, thereby determining the presence or amount of the nucleic acid molecule in the sample.

18. A method of identifying a compound that binds to a compound of claim 1, the method comprising:
(a) contacting the compound with the compound of claim 1; and
(b) determining whether the compound binds to the compound of claim 1.

19. A method of treating or preventing a smooth muscle cell proliferation-associated disorder, the method comprising administering to a subject in which such treatment or prevention is desired the compound of claim 1 in an amount sufficient to treat or prevent the smooth muscle cell proliferation-associated disorder in the subject.

20. The method of claim 19, wherein the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty.

21. The method of claim 19, wherein the subject is a human.

22. A method of treating or preventing a smooth muscle cell proliferation-associated disorder, the method comprising administering to a subject in which such treatment or prevention is desired the nucleic acid of claim 4 in an amount sufficient to treat or prevent the a smooth muscle cell proliferation-associated disorder in the subject.

23. The method of claim 22, wherein the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty.

24. The method of claim 23, wherein the subject is a human.

25. A kit comprising in one or more containers, the pharmaceutical composition of claim 8 and instructions for using the contents therein.

26. A kit comprising in one or more containers, the pharmaceutical composition of claim 12 and instructions for using the contents therein.

27. A kit comprising in one or more containers, the pharmaceutical composition of claim 13 and instructions for using the contents therein.

28. A kit comprising in one or more containers, the pharmaceutical composition of claim 14 and instructions for using the contents therein.

29. A method of treating a pathological state in a mammal, the method comprising administering to the mammal a compound in an amount that is sufficient to alleviate the pathological state, wherein the compound is a compound having an amino acid sequence at least 90% identical to a compound of claim 1.

30. A method of treating a pathological state in a mammal, the method comprising administering to the mammal the antibody of claim 12 in an amount sufficient to alleviate the pathological state.

31. A method of treating a pathological state in a mammal, the method connprising administering to the mammal the virus of claim 6 in an amount sufficient to alleviate the pathological state.

32. A method of treating a smooth muscle cell proliferation-associated disorder in a mammal, the method comprising administering to the mammal at least one compound which modulates the expression or activity of a compound of claim 1.

33. The method of claim 32, wherein the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty.

34. A compound of for use in treating a smooth muscle cell proliferation-associated disorder, wherein the compound is a compound of claim 1.

35. The use of a compound for the manufacture of a medicament for treatment of a smooth muscle cell proliferation-associated disorder, wherein the compound is a compound of claim 1.

36. A method of identifying a compound which binds to a compound of claim 1, the method comprising the steps of:
(a) providing a candidate compound;
(b) contacting the candidate compound with the compound of claim 1 under conditions which a complex is formed between the candidate compound and the compound of claim 1;
(c) incubating the complex under conditions where co-crystals of the complex form;
(d) determining the structural atomic coordinates of the complex by x-ray diffraction; and
(e) modeling the structure of the complex to determine the binding of the candidate compound to the compound of claim 1.

37. A crystalline preparation of a compound and a test compound prepared by the method of claim 36.

38. A method of identifying a compound which binds to a compound of claim 1, the method comprising the steps of:
(a) providing a candidate compound;
(b) contacting the candidate compound with the compound of claim 1 under conditions which a complex is formed between the candidate compound and the compound of claim 1;
(c) determining the binding or structure of the complex by methods of nuclear magnetic resonance spectroscopy or mass; and optionally
(d) modeling the structure of the complex.

39. A device comprising a surface coated with a compound selected from the group consisting of a compound of claim 1, a compound of claim 2, a compound of claim 4, a compound of claim 6, and a compound of claim 9.

40. The device of claim 39, wherein the device is selected from the group consisting of a patch, stent, and catheter.

41. A method of treating a smooth muscle cell proliferation-associated disorder in a mammal, the method comprising contacting a subject with the device of claim 39.

42. The method of claim 41, wherein the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty.

43. The method claim 41, wherein the subject is a human.

44. A compound comprising a parathyroid hormone-related protein mutant polypeptide wherein the compound has a functional nuclear localization signal and has one or more modified amino acids in the region of PTHrP(112-139).

45. The compound of claim 44, wherein the modification of amino acids in the region of PTHrP(112-139) is selected from the group consisting of a deletion, substitution, and derivatization.

46. A compound comprising a parathyroid hormone-related protein mutant peptide wherein the compound has a functional nuclear localization signal and a polypeptide selected from the group consisting of SEQ ID NOS:5, 6, 7, 8, 9, 10, 11, and 12.

47. An isolated nucleic acid encoding the compound of claim 44.

48. A vector comprising the nucleic acid of claim 47.

49. The vector of claim 48, further comprising a promoter operably linked to the nucleic acid molecule.

50. A cell comprising the vector of claim 49.

51. A virus comprising the vector of claim 49.

52. The virus of claim 51, wherein the virus is adenovirus.

53. A pharmaceutical composition comprising a compound of claim 44, and a pharmaceutically acceptable carrier.

54. An antibody or fragment thereof that binds immunospecifically to a compound of claim 44.

55. The antibody of claim 54, wherein the antibody is a monoclonal antibody.

56. The antibody of claim 55, wherein the antibody is a humanized antibody.

57. A pharmaceutical composition comprising an antibody of claim 56, and a pharmaceutically acceptable carrier.

58. A pharmaceutical composition comprising the nucleic acid molecule of claim 49 and a pharmaceutically-acceptable carrier.

59. A pharmaceutical composition comprising the virus of claim 52.

60. A method for preparing a compound, the method comprising:
(a) culturing a cell containing a nucleic acid according to claim 47 under conditions that provide for expression of the compound; and
(b) recovering the expressed compound.

61. A method for determining the presence or amount of the compound of claim 44 in a sample, the method comprising:
(a) providing the sample;
(b) contacting the sample with an antibody that binds immunospecifically to the compound; and
(c) determining the presence or amount of antibody bound to the compound, thereby determining the presence or amount of compound in the sample.

62. A method for determining the presence or amount of the nucleic acid molecule of claim 47 in a sample, the method comprising:
(a) providing the sample;
(b) contacting the sample with a probe that binds to the nucleic acid molecule; and
(c) determining the presence or amount of the probe bound to the nucleic acid molecule, thereby determining the presence or amount of the nucleic acid molecule in the sample.

63. A method of identifying a compound that binds to a compound of claim 44, the method comprising:
(a) contacting the compound with the compound of claim 44; and
(b) determining whether the compound binds to the compound of claim 44.

64. A method of treating or preventing a smooth muscle cell proliferation-associated disorder, the method comprising administering to a subject in which such treatment or prevention is desired the compound of claim 44 in an amount sufficient to treat or prevent the smooth muscle cell proliferation-associated disorder in the subject.

65. The method of claim 64, wherein the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty.

66. The method of claim 64, wherein the subject is a human.

67. A method of treating or preventing a smooth muscle cell proliferation-associated disorder, the method comprising administering to a subject in which such treatment or prevention is desired the nucleic acid of claim 49 in an amount sufficient to treat or prevent the a smooth muscle cell proliferation-associated disorder in the subject.

68. The method of claim 67, wherein the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty.

69. The method of claim 68, wherein the subject is a human.

70. A kit comprising in one or more containers, the pharmaceutical composition of claim 53 and instructions for using the contents therein.

71. A kit comprising in one or more containers, the pharmaceutical composition of claim 57 and instructions for using the contents therein.

72. A kit comprising in one or more containers, the pharmaceutical composition of claim 58 and instructions for using the contents therein.

73. A kit comprising in one or more containers, the pharmaceutical composition of claim 59 and instructions for using the contents therein.

74. A method of treating a pathological state in a mammal, the method comprising administering to the mammal a compound in an amount that is sufficient to alleviate the pathological state, wherein the compound is a compound having an amino acid sequence at least 90% identical to a compound of claim 44.

75. A method of treating a pathological state in a mammal, the method comprising administering to the mammal the antibody of claim 54 in an amount sufficient to alleviate the pathological state.

76. A method of treating a pathological state in a mammal, the method comprising administering to the mammal the virus of claim 51 in an amount sufficient to alleviate the pathological state.

77. A method of treating a smooth muscle cell proliferation-associated disorder in a mammal, the method comprising administering to the mammal at least one compound which modulates the expression or activity of a compound of claim 44.

78. The method of claim 77, wherein the smooth muscle cell proliferation-associated disorder is selected from the group consisting of uterine fibroid tumors, prostatic hypertrophy, bronchial asthma, portal hypertension in cirrhosis, pulmonary arterial hypertension, arterial hypertension, atherosclerosis, bladder disease, and vascular restenosis after angioplasty.

79. A compound of for use in treating a smooth muscle cell proliferation-associated disorder, wherein the compound is a compound of claim 44.

80. The use of a compound for the manufacture of a medicament for treatment of a smooth muscle cell proliferation-associated disorder, wherein the compound is a compound of claim 44.

81. A method of identifying a compound which binds to a compound of claim 44, the method comprising the steps of:
(a) providing a candidate compound;
(b) contacting the candidate compound with the compound of claim 44 under conditions which a complex is formed between the candidate compound and the compound of claim 44;
(c) incubating the complex under conditions where co-crystals or the complex form;
(d) determining the structural atomic coordinates of the complex by x-ray diffraction; and
(e) modeling the structure of the complex to determine the binding of the candidate compound to the compound of claim 44.

82. A crystalline preparation of a compound and a test compound prepared by the method of claim 81.

83. A method of identifying a compound which binds to a compound of claim 44, the method comprising the steps of:
(a) providing a candidate compound;
(b) contacting the candidate compound with the compound of claim 44 under conditions which a complex is formed between the candidate compound and the compound of claim 44;
(c) determining the binding or structure of the complex by methods of nuclear magnetic resonance spectroscopy or mass; and optionally
(d) modeling the structure of the complex.

84. A device comprising a surface coated with a compound selected from the group consisting of a compound of claim 44, a compound of claim 46, a compound of claim 47, a compound of claim 48, a compound of claim 51 and a compound of claim 54.

85. The device of claim 84, wherein the device is selected from the group consisting of a patch, stent, and catheter.

86. A method of treating a smooth muscle cell proliferation-associated disorder in a mammal, the method comprising contacting a subject with the device of claim 84.

87. The method of claim 86, wherein the smooth muscle cell proliferation-associated disorder is selected from the group consisting of uterine fibroid tumors, prostatic hypertrophy, bronchial asthma, portal hypertension in cirrhosis, pulmonary arterial hypertension, systemic arterial hypertension, atherosclerosis, bladder disease, and vascular restenosis after angioplasty.

88. The method claim 86, wherein the subject is a human.
